(19) United States
(12) Patent Application Publication
(10) Pub. No.: US 2020/0203631 A1
(43) Pub. Date: Jun. 25, 2020

(54) ORGANIC ELECTROLUMINESCENT DEVICE COMPRISING A DOPANT MATERIAL AND MULTIPLE HOST MATERIALS

(71) Applicant: BEIJING SUMMER SPROUT TECHNOLOGY CO., LTD., Beijing (CN)

(72) Inventors: Liang GAO, Beijing (CN); Chuanjun XIA, Beijing (CN); Chi Yuen Raymond KWONG, Beijing (CN); Huiqing PANG, Beijing (CN)

(21) Appl. No.: 16/724,989

(22) Filed: Dec. 23, 2019

(30) Foreign Application Priority Data
Dec. 24, 2018 (CN) 201811567753.5

(51) Int. Cl. H01L 51/00; C07D 209/86; C09K 11/06; C07D 405/14; C07F 15/00

(52) U.S. Cl. CPC H01L 51/0072; C07D 209/86; C09K 11/06; C07D 405/14; H01L 51/5012; H01L 51/0067; C07F 15/0033; H01L 51/0085; H01L 51/0073

(57) ABSTRACT

An organic electroluminescent device comprising a dopant material and multiple host materials is disclosed. The device has an emitting layer which comprises two host materials and a dopant material with specific structures. Appropriate energy level matching may be obtained by selecting a combination of particular host materials and a particular dopant material, and the concentration of carriers in the emitting layer may be effectively adjusted to achieve a desired balance, compared with the prior art, the overall performance of the organic light emitting device may be significantly improved. A display module and formulation are also disclosed.

### CROSS-REFERENCE TO RELATED APPLICATIONS

This application claims the benefit of Chinese Application No. 201811567753.5, filed on Dec. 24, 2018 to the China National Intellectual Property Administration, the entire content of which is incorporated herein by reference.

### 1 TECHNICAL FIELD

The present disclosure relates to an organic electroluminescent device. More specifically, the present disclosure relates to an organic electroluminescent device comprising a dopant material and multiple host materials.

### 2 BACKGROUND

An organic electronic device include, but are not limited to, the following group consisting of organic light-emitting devices (OLEDs), organic field-effect transistors (O-FETs), organic light-emitting transistors (OLETs), organic photovoltaic devices (OPVs), dye-sensitized solar cells (DSSCs), organic optical detectors, organic photoreceptors, organic field-quench devices (OFQDs), light-emitting electrochemical cells (LECs), organic laser diodes and organic plasmon emitting devices.

Organic light-emitting devices have many benefits of wide angle, high contrast and faster response time, etc. In 1987, Tang and Van Slyke of Eastman Kodak reported an organic light emitting device, an arylamine hole transporting layer and a tris-8-hydroxyquinolato-aluminum layer as the electron transporting layer and emitting layer (Applied Physics Letters, 1987, 51 (12): 913-915). Once a bias is applied to the device, green light was emitted from the device. This disclosure laid the foundation for the development of modern organic light-emitting diodes (OLEDs). Since OLED is a self-emitting solid state device, it offers tremendous potential for display and lighting applications. In addition, the inherent properties of organic materials, such as their flexibility, may make them well suited for particular applications such as fabrication on flexible substrates.

An organic electroluminescent device converts electrical energy into light by applying a voltage across the device. Generally, an organic electroluminescent device includes an anode, a cathode, and organic layers between the anode and the cathode. The organic layers of the organic electroluminescent device may include a hole injection layer, a hole transporting layer, an electron blocking layer, a light emitting layer (including a host material and a doping material), an electron buffer layer, a hole blocking layer, an electron transporting layer, and an electron injection layer, etc. According to different functions of the materials, the materials constituting the organic layers may be classified into a hole injection material, a hole transporting material, an electron blocking material, a light emitting material, an electron buffering material, a hole blocking material, an electron transporting material, a hole blocking material, and the like. When a bias is applied to the device, holes are injected from the anode to the light-emitting layer, and electrons are injected from the cathode to the light-emitting layer. The holes and electrons meet to form excitons, and the excitons recombine to emit light.

The light-emitting layer material is an important factor affecting the performance of the organic light-emitting device, which needs to have characteristics such as high quantum efficiency, high electron mobility, and high hole mobility. According to the function of materials, the light-emitting layer materials may be divided into host materials and dopant materials. Commercially hoping to obtain organic light-emitting devices having characteristics such as more saturated luminescence spectra, higher efficiencies and longer lifetime, it is important to select a suitable combination of host materials and dopant materials to achieve the above objectives.

U.S. Patent Application No. US20170271598A1 disclosed an organic electroluminescent device using a light emitting layer consisting of two kinds of indolocarbazole type compounds as host materials and a dopant material. This application claims the use of two combinations of host materials with similar molecular structure to obtain similar evaporation temperatures, vapor pressures, and rate of thermal weight loss, thereby achieved the result that the ratio of the two host materials in the mixture before evaporation and the ratio in the film formed after evaporation are close, then get better device performance. This application does not focus on the selection of specific dopant materials for use in combination with the disclosed host materials, only listing a range of existing phosphorescent dopant materials.

U.S. Patent Application No. US20170186965A1 disclosed an organic electroluminescent device using a substituted dibenzofuran compound or a substituted dibenzothiophene compound as a host material and a dopant material to constitute a light-emitting layer. Although the host compound disclosed in this application may be used with more host materials and dopant materials, it does not focus on the selection and matching of multi-component host materials and dopant materials.

US Patent Application No. US20180301636A1 disclosed an organic electroluminescent device comprising multiple host compounds and a phosphorescent dopant as a light-emitting layer, wherein used a combination of host materials, the first host compound has a structure of a nitrogen-containing heterocyclic linker bonded to a nitrogen atom of a carbazole of an indole-carbazole, indene-carbazole, benzofuran-carbazole, or benzothiophene-carbazole residue; and the second host compound has a structure of a carbazole-aryl-carbazole or carbazole-carbazole structure. and claims to obtain an organic electroluminescent device with high efficiency and long service life. However, the first host compound in the host material combination is the compound with an indole-carbazole, an indene-oxazole, a benzofuran-carbazole or a benzothiophene-carbazole which must include a nitrogen-containing heterocyclic linker to which a carbazole nitrogen atom is bonded, thereby changing its ability to match the energy levels of other materials, thereby affecting the performance of the electroluminescent device. Moreover, this application does not focus on the selection of specific dopant materials used in combination with the disclosed host material, only listed a range of existing phosphorescent dopant materials. The overall performance of devices implemented with specific dopant materials also needs to be improved.

The Chinese Patent Application No. CN201811071665.6 is a prior patent application of the present applicant, the entire contents of which is hereby incorporated by reference. This application disclosed a metal complex as a light emitting dopant, although it is mentioned that the dopant may be used with one or more existing host materials, it only lists a series of existing host materials. The application does not focus on the coordination of the dopant with a variety of host materials of a specific structure.

The inventors have found through intensive studies that a multi-component host material combination which combine a compound containing a carbazole-carbazole structure used as the first host material with a compound having a carbazole-dibenzofuran-(hetero)aryl, carbazole-dibenzothiophene-(hetero)aryl structure as the second host compound, cooperating with a light-emitting layer dopant material having a specific iridium complex structure as a light-emitting layer, may significantly improve the overall performance of organic electroluminescent devices.

### 3 SUMMARY

The present disclosure aims to provide an organic electroluminescent device to solve at least part of the above problems. For this purpose, an organic electroluminescent device comprising a combination of specific host materials and a dopant material is provided, the combination of particular host materials and a particular dopant material results in a suitable energy level matching, effectively regulate the concentration of carriers in the light-emitting layer to achieve the desired balance, thereby improving the overall performance of the organic light-emitting device.

According to an embodiment of the present disclosure, an organic electroluminescent device is disclosed, which comprises:

an anode,
a cathode,
and at least one light emitting layer between the anode and the cathode, the light emitting layer comprises host materials and a dopant material, wherein the host materials comprises a first host compound represented by formula 1 and a second host compound represented by formula 2, the dopant material comprises a dopant compound represented by formula 3:

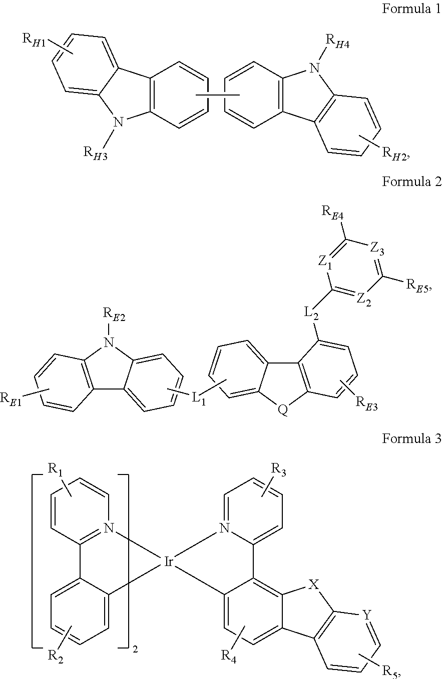

Wherein,

R_{H1}, R_{H2}, R_{E1}, R_{E3}, R₁ to R₅ may represent mono substitution, multiple substitutions or no substitution; when R_{H1}, R_{H2}, R_{E1}, R_{E3}, R₁ to R₅ represent multiple substitutions, each of R_{H1}, R_{H2}, R_{E1}, R_{E3}, R₁ to R₅ may be the same or different;

X and Q are each independently selected from O or S;

Z₁, Z₂, Z₃ and Y are each independently selected from CR or N;

L₁ and L₂ are each independently selected from the group consisting of a single bond, a substituted or unsubstituted arylene having 6 to 30 carbon atoms, a substituted or unsubstituted heteroarylene having 3 to 30 carbon atoms, and combinations thereof;

R₁ to R₅, R_{H1} to R_{H4}, R_{E1} to R_{E5} and R are each independently selected from the group consisting of hydrogen, deuterium, halogen, a substituted or unsubstituted alkyl group having 1 to 20 carbon atoms, a substituted or unsubstituted cycloalkyl group having 3 to 20 ring carbon atoms, a substituted or unsubstituted heteroalkyl group having 1 to 20 carbon atoms, a substituted or unsubstituted arylalkyl group having 7 to 30 carbon atoms, a substituted or unsubstituted alkoxy group having 1 to 20 carbon atoms, a substituted or unsubstituted aryloxy group having 6 to 30 carbon atoms, a substituted or unsubstituted alkenyl group having 2 to 20 carbon atoms, a substituted or unsubstituted aryl group having 6 to 30 carbon atoms, a substituted or unsubstituted heteroaryl group having 3 to 30 carbon atoms, a substituted or unsubstituted alkylsilyl group having 3 to 20 carbon atoms, a substituted or unsubstituted arylsilyl group having 6 to 20 carbon atoms, a substituted or unsubstituted amino group having 0 to 20 carbon atoms, an acyl group, a carbonyl group, a carboxylic acid group, an ester group, a nitrile group, an isonitrile group, a thiolalkyl group, a sulfinyl group, a sulfonyl group, a phosphino group, and combinations thereof;

adjacent substitutions may be optionally joined to form a ring.

According to another embodiment of the present disclosure, a display module is also disclosed, which comprises the organic electroluminescent device.

According to yet another embodiment of the present disclosure, a formulation is also disclosed, which comprises host materials and a dopant material, wherein the host materials comprises a first host compound represented by formula 1 and a second host compound represented by formula 2, wherein the dopant material comprises a dopant compound represented by formula 3:

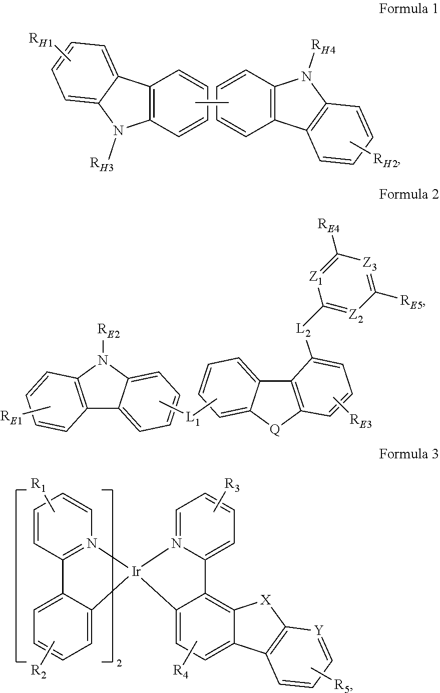

wherein,

R_{H1}, R_{H2}, R_{E1}, R_{E3}, R₁ to R₅ may represent mono substitution, multiple substitutions or no substitution; when R_{H1}, R_{H2}, R_{E1}, R_{E3}, R₁ to R₅ represent multiple substitution, each of R_{H1}, R_{H2}, R_{E1}, R_{E3}, R₁ to R₅ may be the same or different;

X and Q are each independently selected from O or S;

Z₁, Z₂, Z₃ and Y are each independently selected from CR or N;

L₁ and L₂ are each independently selected from the group consisting of a single bond, a substituted or unsubstituted arylene having 6 to 30 carbon atoms, a substituted or unsubstituted heteroarylene having 3 to 30 carbon atoms, and combinations thereof;

R₁ to R₅, R_{H1} to R_{H4}, R_{E1} to R_{E5} and R are each independently selected from the group consisting of hydrogen, deuterium, halogen, a substituted or unsubstituted alkyl group having 1 to 20 carbon atoms, a substituted or unsubstituted cycloalkyl group having 3 to 20 ring carbon atoms, a substituted or unsubstituted heteroalkyl group having 1 to 20 carbon atoms, a substituted or unsubstituted arylalkyl group having 7 to 30 carbon atoms, a substituted or unsubstituted alkoxy group having 1 to 20 carbon atoms, a substituted or unsubstituted aryloxy group having 6 to 30 carbon atoms, a substituted or unsubstituted alkenyl group having 2 to 20 carbon atoms, a substituted or unsubstituted aryl group having 6 to 30 carbon atoms, a substituted or unsubstituted heteroaryl group having 3 to 30 carbon atoms, a substituted or unsubstituted alkylsilyl group having 3 to 20 carbon atoms, a substituted or unsubstituted arylsilyl group having 6 to 20 carbon atoms, a substituted or unsubstituted amino group having 0 to 20 carbon atoms, an acyl group, a carbonyl group, a carboxylic acid group, an ester group, a nitrile group, an isonitrile group, a thiolalkyl group, a sulfinyl group, a sulfonyl group, a phosphino group, and combinations thereof;

any adjacent substitution may be optionally joined to form a ring.

The organic electroluminescent device comprising a dopant material and plurality of host materials is disclosed in the present disclosure, wherein the light emitting layer comprises two host materials and a dopant material with specific structures. By selecting a particular combination of host compounds and a dopant compound, a suitable energy level matching of the emitting layer material may be obtained, effectively regulate the concentration of carriers in the emitting layer to achieve the desired balance, compared to the prior art, the performance of organic electroluminescent devices improved significantly, such as lifetime, voltage and luminous efficiency have improved.

### 4 BRIEF DESCRIPTION OF THE DRAWINGS

FIG. 1 schematically shows an organic electroluminescent device.

FIG. 2 schematically shows another organic electroluminescent device.

### 5 DETAILED DESCRIPTION

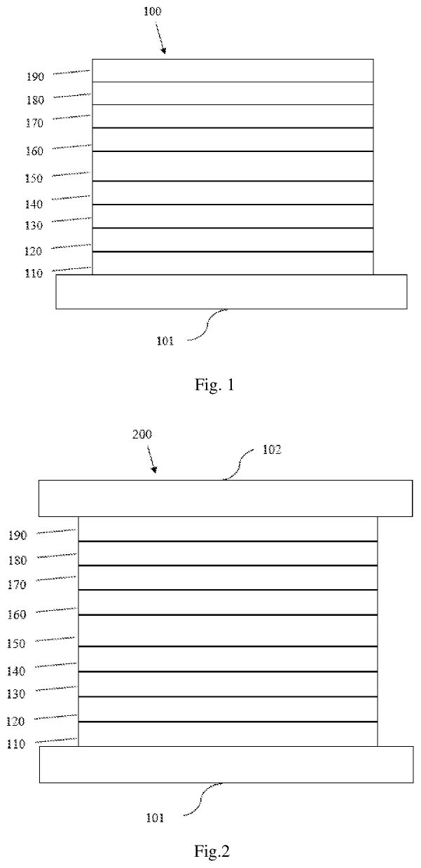

OLEDs may be fabricated on various types of substrates such as glass, plastic, and metal foil. FIG. 1 schematically shows the organic light emitting device 100 without limitation. The figures are not necessarily drawn to scale. Some of the layers in the figures may also be omitted as needed. Device 100 may include a substrate 101, an anode 110, a hole injection layer 120, a hole transport layer 130, an electron blocking layer 140, an emissive layer 150, a hole blocking layer 160, an electron transport layer 170, an electron injection layer 180 and a cathode 190. Device 100 may be fabricated by depositing the layers described in order. The properties and functions of these various layers, as well as example materials, are described in more detail in U.S. Pat. No. 7,279,704 at cols. 6-10, the contents of which are incorporated by reference herein in its entirety.

More examples for each of these layers are available. For example, a flexible and transparent substrate-anode combination is disclosed in U.S. Pat. No. 5,844,363, which is incorporated by reference herein in its entirety. An example of a p-doped hole transport layer is m-MTDATA doped with F₄-TCNQ at a molar ratio of 50:1, as disclosed in U.S. Patent Application Publication No. 2003/0230980, which is incorporated by reference herein in its entirety. Examples of host materials are disclosed in U.S. Pat. No. 6,303,238 to Thompson et al., which is incorporated by reference herein in its entirety. An example of an n-doped electron transport layer is BPhen doped with Li at a molar ratio of 1:1, as disclosed in U.S. Patent Application Publication No. 2003/0230980, which is incorporated by reference herein in its entirety. U.S. Pat. Nos. 5,703,436 and 5,707,745, which are incorporated by reference herein in their entireties, disclose examples of cathodes including composite cathodes having a thin layer of metal such as Mg:Ag with an overlying transparent, electrically-conductive, sputter-deposited ITO layer. The theory and use of blocking layers is described in more detail in U.S. Pat. No. 6,097,147 and U.S. Patent Application Publication No. 2003/0230980, which are incorporated by reference herein in their entireties. Examples of injection layers are provided in U.S. Patent Application Publication No. 2004/0174116, which is incorporated by reference herein in its entirety. A description of protective layers may be found in U.S. Patent Application Publication No. 2004/0174116, which is incorporated by reference herein in its entirety.

The layered structure described above is provided by way of non-limiting example. Functional OLEDs may be achieved by combining the various layers described in different ways, or layers may be omitted entirely. It may also include other layers not specifically described. Within each layer, a single material or a mixture of multiple materials may be used to achieve optimum performance. Any functional layer may include several sublayers. For example, the emissive layer may have two layers of different emitting materials to achieve desired emission spectrum.

In one embodiment, an OLED may be described as having an “organic layer” disposed between a cathode and an anode. This organic layer may comprise a single layer or multiple layers.

An OLED may be encapsulated by a barrier layer. FIG. 2 schematically shows the organic light emitting device 200 without limitation. FIG. 2 differs from FIG. 1 in that the organic light emitting device include a barrier layer 102, which is above the cathode 190, to protect it from harmful species from the environment such as moisture and oxygen. Any material that may provide the barrier function may be used as the barrier layer such as glass and organic-inorganic hybrid layers. The barrier layer should be placed directly or indirectly outside of the OLED device. Multilayer thin film encapsulation was described in U.S. Pat. No. 7,968,146, which is herein incorporated by reference in its entirety.

Devices fabricated in accordance with embodiments of the present disclosure may be incorporated into a wide variety of consumer products that have one or more of the electronic component modules (or units) incorporated therein. Some examples of such consumer products include flat panel displays, monitors, medical monitors, televisions, billboards, lights for interior or exterior illumination and/or signaling, heads-up displays, fully or partially transparent displays, flexible displays, smart phones, tablets, phablets, wearable devices, smart watches, laptop computers, digital cameras, camcorders, viewfinders, micro-displays, 3-D displays, vehicles displays, and vehicle tail lights.

The materials and structures described herein may be used in other organic electronic devices listed above.

As used herein, “top” means furthest away from the substrate, while “bottom” means closest to the substrate. Where a first layer is described as “disposed over” a second layer, the first layer is disposed further away from substrate. There may be other layers between the first and second layer, unless it is specified that the first layer is “in contact with” the second layer. For example, a cathode may be described as “disposed over” an anode, even though there are various organic layers in between.

As used herein, “solution processible” means capable of being dissolved, dispersed, or transported in and/or deposited from a liquid medium, either in solution or suspension form.

A ligand may be referred to as “photoactive” when it is believed that the ligand directly contributes to the photoactive properties of an emissive material. A ligand may be referred to as “ancillary” when it is believed that the ligand does not contribute to the photoactive properties of an emissive material, although an ancillary ligand may alter the properties of a photoactive ligand.

It is believed that the internal quantum efficiency (IQE) of fluorescent OLEDs may exceed the 25% spin statistics limit through delayed fluorescence. As used herein, there are two types of delayed fluorescence, i.e. P-type delayed fluorescence and E-type delayed fluorescence. P-type delayed fluorescence is generated from triplet-triplet annihilation (TTA).

On the other hand, E-type delayed fluorescence does not rely on the collision of two triplets, but rather on the transition between the triplet states and the singlet excited states. Compounds that are capable of generating E-type delayed fluorescence are required to have very small singlet-triplet gaps to convert between energy states. Thermal energy may activate the transition from the triplet state back to the singlet state. This type of delayed fluorescence is also known as thermally activated delayed fluorescence (TADF). A distinctive feature of TADF is that the delayed component increases as temperature rises. If the reverse intersystem crossing rate is fast enough to minimize the non-radiative decay from the triplet state, the fraction of back populated singlet excited states may potentially reach 75%. The total singlet fraction may be 100%, far exceeding 25% of the spin statistics limit for electrically generated excitons.

E-type delayed fluorescence characteristics may be found in an exciplex system or in a single compound. Without being bound by theory, it is believed that E-type delayed fluorescence requires the luminescent material to have a small singlet-triplet energy gap (ΔE_{S-T}). Organic, non-metal containing, donor-acceptor luminescent materials may be able to achieve this. The emission in these materials is often characterized as a donor-acceptor charge-transfer (CT) type emission. The spatial separation of the HOMO and LUMO in these donor-acceptor type compounds often results in small ΔE_{S-T}. These states may involve CT states. Often, donor-acceptor luminescent materials are constructed by connecting an electron donor moiety (such as amino- or carbazole-derivatives) and an electron acceptor moiety (such as N-containing six-membered aromatic rings).

### Definition of Terms of Substituents

halogen or halide—as used herein includes fluorine, chlorine, bromine, and iodine.

Alkyl—contemplates both straight and branched chain alkyl groups. Examples of the alkyl group include methyl group, ethyl group, propyl group, isopropyl group, n-butyl group, s-butyl group, isobutyl group, t-butyl group, n-pentyl group, n-hexyl group, n-heptyl group, n-octyl group, n-nonyl group, n-decyl group, n-undecyl group, n-dodecyl group, n-tridecyl group, n-tetradecyl group, n-pentadecyl group, n-hexadecyl group, n-heptadecyl group, n-octadecyl group, neopentyl group, 1-methylpentyl group, 2-methylpentyl group, 1-pentylhexyl group, 1-butylpentyl group, 1-heptyloctyl group, and 3-methylpentyl group. Additionally, the alkyl group may be optionally substituted. The carbons in the alkyl chain may be replaced by other hetero atoms. Of the above, preferred are methyl group, ethyl group, propyl group, isopropyl group, n-butyl group, s-butyl group, isobutyl group, t-butyl group, n-pentyl group, and neopentyl group.

Cycloalkyl—as used herein contemplates cyclic alkyl groups. Preferred cycloalkyl groups are those containing 4 to 10 ring carbon atoms and includes cyclobutyl, cyclopentyl, cyclohexyl, 4-methylcyclohexyl, 4,4-dimethylcylcohexyl, 1-adamantyl, 2-adamantyl, 1-norbornyl, 2-norbornyl and the like. Additionally, the cycloalkyl group may be optionally substituted. The carbons in the ring can be replaced by other hetero atoms.

Alkenyl—as used herein contemplates both straight and branched chain alkene groups. Preferred alkenyl groups are those containing 2 to 15 carbon atoms. Examples of the alkenyl group include vinyl group, allyl group, 1-butenyl group, 2-butenyl group, 3-butenyl group, 1,3-butandienyl group, 1-methylvinyl group, styryl group, 2,2-diphenylvinyl group, 1,2-diphenylvinyl group, 1-methylallyl group, 1,1-dimethylallyl group, 2-methylallyl group, 1-phenylallyl group, 2-phenylallyl group, 3-phenylallyl group, 3,3-diphenylallyl group, 1,2-dimethylallyl group, 1-phenyl1-butenyl group, and 3-phenyl-1-butenyl group. Additionally, the alkenyl group may be optionally substituted.

Alkynyl—as used herein contemplates both straight and branched chain alkyne groups. Preferred alkynyl groups are those containing 2 to 15 carbon atoms. Additionally, the alkynyl group may be optionally substituted.

Aryl or aromatic group—as used herein includes noncondensed and condensed systems. Preferred aryl groups are those containing six to sixty carbon atoms, preferably six to twenty carbon atoms, more preferably six to twelve carbon atoms. Examples of the aryl group include phenyl, biphenyl, terphenyl, triphenylene, tetraphenylene, naphthalene, anthracene, phenalene, phenanthrene, fluorene, pyrene, chrysene, perylene, and azulene, preferably phenyl, biphenyl, terphenyl, triphenylene, fluorene, and naphthalene. Additionally, the aryl group may be optionally substituted. Examples of the non-condensed aryl group include phenyl group, biphenyl-2-yl group, biphenyl-3-yl group, biphenyl-4-yl group, p-terphenyl-4-yl group, p-terphenyl-3-yl group, p-terphenyl-2-yl group, m-terphenyl-4-yl group, m-terphenyl-3-yl group, m-terphenyl-2-yl group, o-tolyl group, m-tolyl group, p-tolyl group, p-t-butylphenyl group, p-(2-phenylpropyl)phenyl group, 4′-methylbiphenylyl group, 4″-t-butyl p-terphenyl-4-yl group, o-cumenyl group, m-cumenyl group, p-cumenyl group, 2,3-xylyl group, 3,4-xylyl group, 2,5-xylyl group, mesityl group, and m-quarterphenyl group.

Heterocyclic group or heterocycle—as used herein includes aromatic and non-aromatic cyclic groups. Hetero-aromatic also means heteroaryl. Preferred non-aromatic heterocyclic groups are those containing 3 to 7 ring atoms which include at least one hetero atom such as nitrogen, oxygen, and sulfur. The heterocyclic group may also be an aromatic heterocyclic group having at least one heteroatom selected from nitrogen atom, oxygen atom, sulfur atom, and selenium atom.

Heteroaryl—as used herein includes noncondensed and condensed hetero-aromatic groups that may include from one to five heteroatoms. Preferred heteroaryl groups are those containing three to thirty carbon atoms, preferably three to twenty carbon atoms, more preferably three to twelve carbon atoms. Suitable heteroaryl groups include dibenzothiophene, dibenzofuran, dibenzoselenophene, furan, thiophene, benzofuran, benzothiophene, benzoselenophene, carbazole, indolocarbazole, pyridylindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indoxazine, benzoxazole, benzisoxazole, benzothiazole, quinoline, isoquinoline, cinnoline, quinazoline, quinoxaline, naphthyridine, phthalazine, pteridine, xanthene, acridine, phenazine, phenothiazine, phenoxazine, benzofuropyridine, furodipyridine, benzothienopyridine, thienodipyridine, benzoselenophenopyridine, and selenophenodipyridine, preferably dibenzothiophene, dibenzofuran, dibenzoselenophene, carbazole, indolocarbazole, imidazole, pyridine, triazine, benzimidazole, 1,2-azaborine, 1,3-azaborine, 1,4-azaborine, borazine, and aza-analogs thereof. Additionally, the heteroaryl group may be optionally substituted.

Alkoxy—it is represented by —O-Alkyl. Examples and preferred examples thereof are the same as those described above. Examples of the alkoxy group having 1 to 20 carbon atoms, preferably 1 to 6 carbon atoms include methoxy group, ethoxy group, propoxy group, butoxy group, pentyloxy group, and hexyloxy group. The alkoxy group having 3 or more carbon atoms may be linear, cyclic or branched.

Aryloxy—it is represented by —O-Aryl or —O-heteroaryl. Examples and preferred examples thereof are the same as those described above. Examples of the aryloxy group having 6 to 40 carbon atoms include phenoxy group and biphenyloxy group.

Arylalkyl—as used herein contemplates an alkyl group that has an aryl substituent. Additionally, the arylalkyl group may be optionally substituted. Examples of the arylalkyl group include benzyl group, 1-phenylethyl group, 2-phenylethyl group, 1-phenylisopropyl group, 2-phenylisopropyl group, phenyl-t-butyl group, alpha.-naphthylmethyl group, 1-alpha.-naphthylethyl group, 2-alpha-naphthylethyl group, 1-alpha-naphthylisopropyl group, 2-alpha-naphthylisopropyl group, beta-naphthylmethyl group, 1-beta-naphthylethyl group, 2-beta-naphthylethyl group, 1-beta-naphthylisopropyl group, 2-beta-naphthylisopropyl group, p-methylbenzyl group, m-methylbenzyl group, o-methylbenzyl group, p-chlorobenzyl group, m-chlorobenzyl group, o-chlorobenzyl group, p-bromobenzyl group, m-bromobenzyl group, o-bromobenzyl group, p-iodobenzyl group, m-iodobenzyl group, o-iodobenzyl group, p-hydroxybenzyl group, m-hydroxybenzyl group, o-hydroxybenzyl group, p-aminobenzyl group, m-aminobenzyl group, o-aminobenzyl group, p-nitrobenzyl group, m-nitrobenzyl group, o-nitrobenzyl group, p-cyanobenzyl group, m-cyanobenzyl group, o-cyanobenzyl group, 1-hydroxy-2-phenylisopropyl group, and 1-chloro-2-phenylisopropyl group. Of the above, preferred are benzyl group, p-cyanobenzyl group, m-cyanobenzyl group, o-cyanobenzyl group, 1-phenylethyl group, 2-phenylethyl group, 1-phenylisopropyl group, and 2-phenylisopropyl group.

The term “aza” in azadibenzofuran, aza-dibenzothiophene, etc. means that one or more of the C—H groups in the respective aromatic fragment are replaced by a nitrogen atom. For example, azatriphenylene encompasses dibenzo[f,h]quinoxaline, dibenzo[f,h]quinoline and other analogues with two or more nitrogens in the ring system. One of ordinary skill in the art may readily envision other nitrogen analogs of the aza-derivatives described above, and all such analogs are intended to be encompassed by the terms as set forth herein.

The alkyl, cycloalkyl, alkenyl, alkynyl, aralkyl, heterocyclic group, aryl, and heteroaryl may be unsubstituted or may be substituted with one or more substituents selected from the group consisting of deuterium, halogen, alkyl, cycloalkyl, arylalkyl, alkoxy, aryloxy, amino, cyclic amino, silyl, alkenyl, cycloalkenyl, heteroalkenyl, alkynyl, aryl, heteroaryl, an acyl group, a carbonyl group, a carboxylic acid group, an ether group, an ester group, a nitrile group, an isonitrile group, a thiolalkyl group, a sulfinyl group, a sulfonyl group, a phosphino group, and combinations thereof.

It is to be understood that when a molecular fragment is described as being a substituent or otherwise attached to another moiety, its name may be written as if it were a fragment (e.g. phenyl, phenylene, naphthyl, dibenzofuryl) or as if it were the whole molecule (e.g. benzene, naphthalene, dibenzofuran). As used herein, these different ways of designating a substituent or attached fragment are considered to be equivalent.

In the compounds mentioned in this disclosure, the hydrogen atoms may be partially or fully replaced by deuterium. Other atoms, such as carbon and nitrogen may also be replaced by their other stable isotopes. The replacement by other stable isotopes in the compounds may be preferred due to its enhancements of device efficiency and stability.

In the compounds mentioned in this disclosure, multiple substitutions refer to a range that includes a double substitution, up to the maximum available substitutions.

In the compounds mentioned in this disclosure, the expression that adjacent substitutions may be optionally joined to form a ring is intended to be taken to mean that two radicals are linked to each other by a chemical bond. This is illustrated by the following scheme:

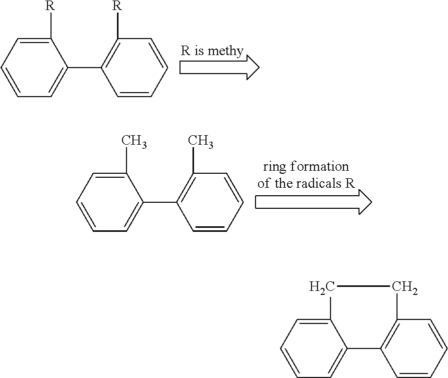

Furthermore, the expression that adjacent substitutions may be optionally joined to form a ring is also intended to be taken to mean that in the case where one of the two radicals represents hydrogen, the second radical is bonded at a position to which the hydrogen atom was bonded, with formation of a ring. This is illustrated by the following scheme:

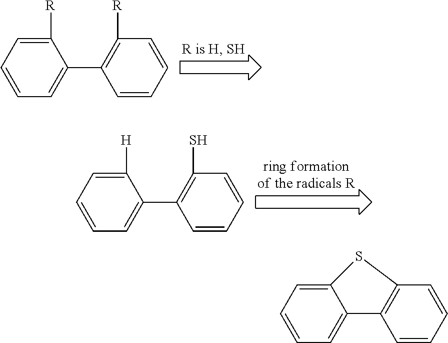

According to an embodiment of the present disclosure, an organic electroluminescent device is disclosed, which comprises:

an anode,

a cathode,

and at least one light emitting layer between the anode and the cathode, the light emitting layer comprises host materials and a dopant material, wherein the host materials comprises a first host compound represented by formula 1 and a second host compound represented by formula 2, the dopant material comprises a dopant compound represented by formula 3:

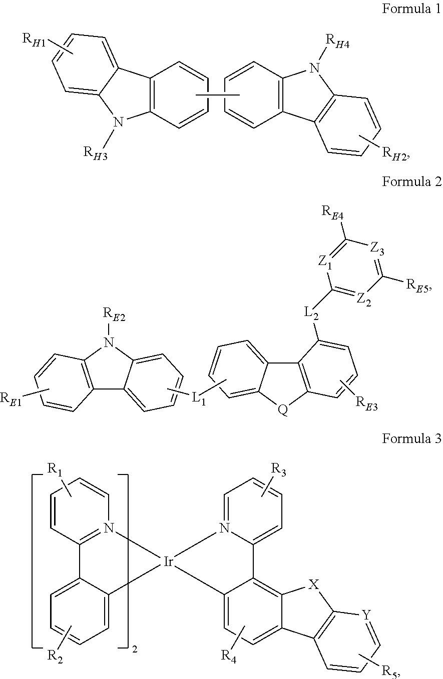

Wherein,

R_{H1}, R_{H2}, R_{E1}, R_{E3}, R₁ to R₅ may represent mono substitution, multiple substitutions or no substitution; when R_{H1}, R_{H2}, R_{E1}, R_{E3}, R₁ to R₅ represent multiple substitution, each of R_{H1}, R_{H2}, R_{E1}, R_{E3}, R₁ to R₅ may be the same or different;

X and Q are each independently selected from O or S;

Z₁, Z₂, Z₃ and Y are each independently selected from CR or N;

L₁ and L₂ are each independently selected from the group consisting of a single bond, a substituted or unsubstituted arylene having 6 to 30 carbon atoms, a substituted or unsubstituted heteroarylene having 3 to 30 carbon atoms, and combinations thereof;

R₁ to R₅, R_{H1} to R_{H4}, R_{E1} to R_{E5} and R are each independently selected from the group consisting of hydrogen, deuterium, halogen, a substituted or unsubstituted alkyl group having 1 to 20 carbon atoms, a substituted or unsubstituted cycloalkyl group having 3 to 20 ring carbon atoms, a substituted or unsubstituted heteroalkyl group having 1 to 20 carbon atoms, a substituted or unsubstituted arylalkyl group having 7 to 30 carbon atoms, a substituted or unsubstituted alkoxy group having 1 to 20 carbon atoms, a substituted or unsubstituted aryloxy group having 6 to 30 carbon atoms, a substituted or unsubstituted alkenyl group having 2 to 20 carbon atoms, a substituted or unsubstituted aryl group having 6 to 30 carbon atoms, a substituted or unsubstituted heteroaryl group having 3 to 30 carbon atoms, a substituted or unsubstituted alkylsilyl group having 3 to 20 carbon atoms, a substituted or unsubstituted arylsilyl group having 6 to 20 carbon atoms, a substituted or unsubstituted amino group having 0 to 20 carbon atoms, an acyl group, a carbonyl group, a carboxylic acid group, an ester group, a nitrile group, an isonitrile group, a thiolalkyl group, a sulfinyl group, a sulfonyl group, a phosphino group, and combinations thereof;

adjacent substitutions may be optionally joined to form a ring.

R_{H1}, R_{H2}, R_{E1}, R_{E3}, R₁ to R₅ may represent mono substitution, multiple substitutions or no substitution, taking R₃ as an example, an exemplary description is as follows: when R₃ represents mono substitution, it means that there is only one R₃ substituent on the ring to which R₃ is bonded, and its bonding position is any substitutable position of the bonded ring; when R₃ represents multiple substitutions, it means that there may be a plurality of R₃ substituents on the ring to which R₃ is bonded, and the number of R₃ may be arbitrarily selected from two to up to the maximum of four available substitutions, and the plurality of R₃ substituents may be the same or different, for example, when R₃ represents double substitutions, one of R₃ may be methyl, another R₃ may be isopropyl, the bonding position of methyl and isopropyl may be any substitutable position of the bonded ring.

According to an embodiment of the present disclosure, wherein the first host compound having the structure represent by formula 4:

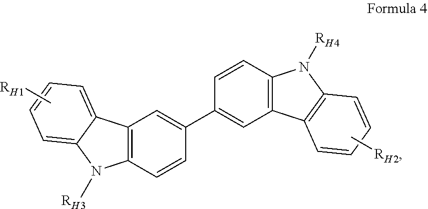

wherein R_{H1} and R_{H2} may represent mono substitution, multiple substitutions or no substitution; when R_{H1}, R_{H2} represent multiple substitutions, each of R_{H1}, R_{H2} may be the same or different;

R_{H1} to R_{H4} are each independently selected from the group consisting of hydrogen, deuterium, halogen, a substituted or unsubstituted alkyl group having 1 to 20 carbon atoms, a substituted or unsubstituted cycloalkyl group having 3 to 20 ring carbon atoms, a substituted or unsubstituted heteroalkyl group having 1 to 20 carbon atoms, a substituted or unsubstituted arylalkyl group having 7 to 30 carbon atoms, a substituted or unsubstituted alkoxy group having 1 to 20 carbon atoms, a substituted or unsubstituted aryloxy group having 6 to 30 carbon atoms, a substituted or unsubstituted alkenyl group having 2 to 20 carbon atoms, a substituted or unsubstituted aryl group having 6 to 30 carbon atoms, a substituted or unsubstituted heteroaryl group having 3 to 30 carbon atoms, a substituted or unsubstituted alkylsilyl group having 3 to 20 carbon atoms, a substituted or unsubstituted arylsilyl group having 6 to 20 carbon atoms, a substituted or unsubstituted amino group having 0 to 20 carbon atoms, an acyl group, a carbonyl group, a carboxylic acid group, an ester group, a nitrile group, an isonitrile group, a thiolalkyl group, a sulfinyl group, a sulfonyl group, a phosphino group, and combinations thereof.

According to an embodiment of the present disclosure, wherein R_{H1} to R_{H4} are each independently selected from the group consisting of hydrogen, deuterium, a substituted or unsubstituted aryl group having 6 to 30 carbon atoms, a substituted or unsubstituted heteroaryl group having 3 to 30 carbon atoms, and combinations thereof.

According to an embodiment of the present disclosure, R_{H1} to R_{H4} are each independently selected from the group consisting of hydrogen, deuterium, phenyl, biphenyl, terphenyl, naphthyl, phenanthryl, triphenylene, deuterated phenyl, deuterated biphenyl, deuterated terphenyl, deuterated naphthyl, deuterated phenanthrenyl, deuterated triphenylene, and combinations thereof.

According to an embodiment of the present disclosure, wherein the second host compound having the structure represent by formula 5:

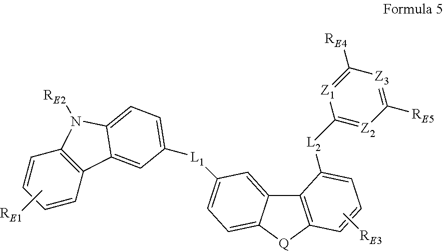

wherein, R_{E1} and R_{E3} may represent mono substitution, multiple substitutions or no substitution; when R_{E1}, R_{E3} represent multiple substitutions, each of R_{E1}, R_{E3} may be the same or different;

Q is selected from O or S;

Z₁, Z₂ and Z₃ are each independently selected from CR or N;

L₁ and L₂ are each independently selected from the group consisting of a single bond, a substituted or unsubstituted arylene having 6 to 30 carbon atoms, a substituted or unsubstituted heteroarylene having 3 to 30 carbon atoms, and combinations thereof;

R_{E1} to R_{E5} are each independently selected from the group consisting of hydrogen, deuterium, halogen, a substituted or unsubstituted alkyl group having 1 to 20 carbon atoms, a substituted or unsubstituted cycloalkyl group having 3 to 20 ring carbon atoms, a substituted or unsubstituted heteroalkyl group having 1 to 20 carbon atoms, a substituted or unsubstituted arylalkyl group having 7 to 30 carbon atoms, a substituted or unsubstituted alkoxy group having 1 to 20 carbon atoms, a substituted or unsubstituted aryloxy group having 6 to 30 carbon atoms, a substituted or unsubstituted alkenyl group having 2 to 20 carbon atoms, a substituted or unsubstituted aryl group having 6 to 30 carbon atoms, a substituted or unsubstituted heteroaryl group having 3 to 30 carbon atoms, a substituted or unsubstituted alkylsilyl group having 3 to 20 carbon atoms, a substituted or unsubstituted arylsilyl group having 6 to 20 carbon atoms, a substituted or unsubstituted amino group having 0 to 20 carbon atoms, an acyl group, a carbonyl group, a carboxylic acid group, an ester group, a nitrile group, an isonitrile group, a thiolalkyl group, a sulfinyl group, a sulfonyl group, a phosphino group, and combinations thereof;

any adjacent substitution may be optionally joined to form a ring.

According to an embodiment of the present disclosure, wherein R_{E1} to R_{E5} are each independently selected from the group consisting of hydrogen, deuterium, a substituted or unsubstituted aryl group having 6 to 30 carbon atoms, a substituted or unsubstituted heteroaryl group having 3 to 30 carbon atoms, and combinations thereof.

According to an embodiment of the present disclosure, wherein R_{E1} represents multiple substitutions, and adjacent R_{E1} may be optionally joined to form a ring.

According to an embodiment of the present disclosure, wherein R_{E1} to R_{E5} are each independently selected from the group consisting of hydrogen, deuterium, phenyl, biphenyl, fluorenyl, dibenzofuranyl, naphthyl, deuterated phenyl, deuterated biphenyl, deuterated fluorenyl, deuterated dibenzofuranyl, deuterated naphthyl, and combinations thereof.

According to an embodiment of the present disclosure, wherein Z₁ to Z₃ are all N.

According to an embodiment of the present disclosure, wherein L₁ and L₂ are each independently selected from the group consisting of a single bond, a substituted or unsubstituted arylene having 6 to 20 carbon atoms, a substituted or unsubstituted heteroarylene having 3 to 15 carbon atoms, and combinations thereof.

According to an embodiment of the present disclosure, wherein L₁ and L₂ are each independently selected from a single bond or phenylene.

According to an embodiment of the present disclosure, wherein X is O.

According to an embodiment of the present disclosure, wherein Y is CH, CD or N.

According to an embodiment of the present disclosure, wherein R₁ to R₅ are each independently selected from the group consisting of hydrogen, deuterium, halogen, a substituted or unsubstituted alkyl group having 1 to 20 carbon atoms, a substituted or unsubstituted cycloalkyl group having 3 to 20 ring carbon atoms, and combinations thereof.

According to an embodiment of the present disclosure, wherein R₁ to R₅ are each independently selected from the group consisting of hydrogen, deuterium, fluorine, methyl, isopropyl, deuterated methyl, deuterated isopropyl, and combinations thereof.

According to an embodiment of the present disclosure, wherein the first host compound is selected from the group consisting of:

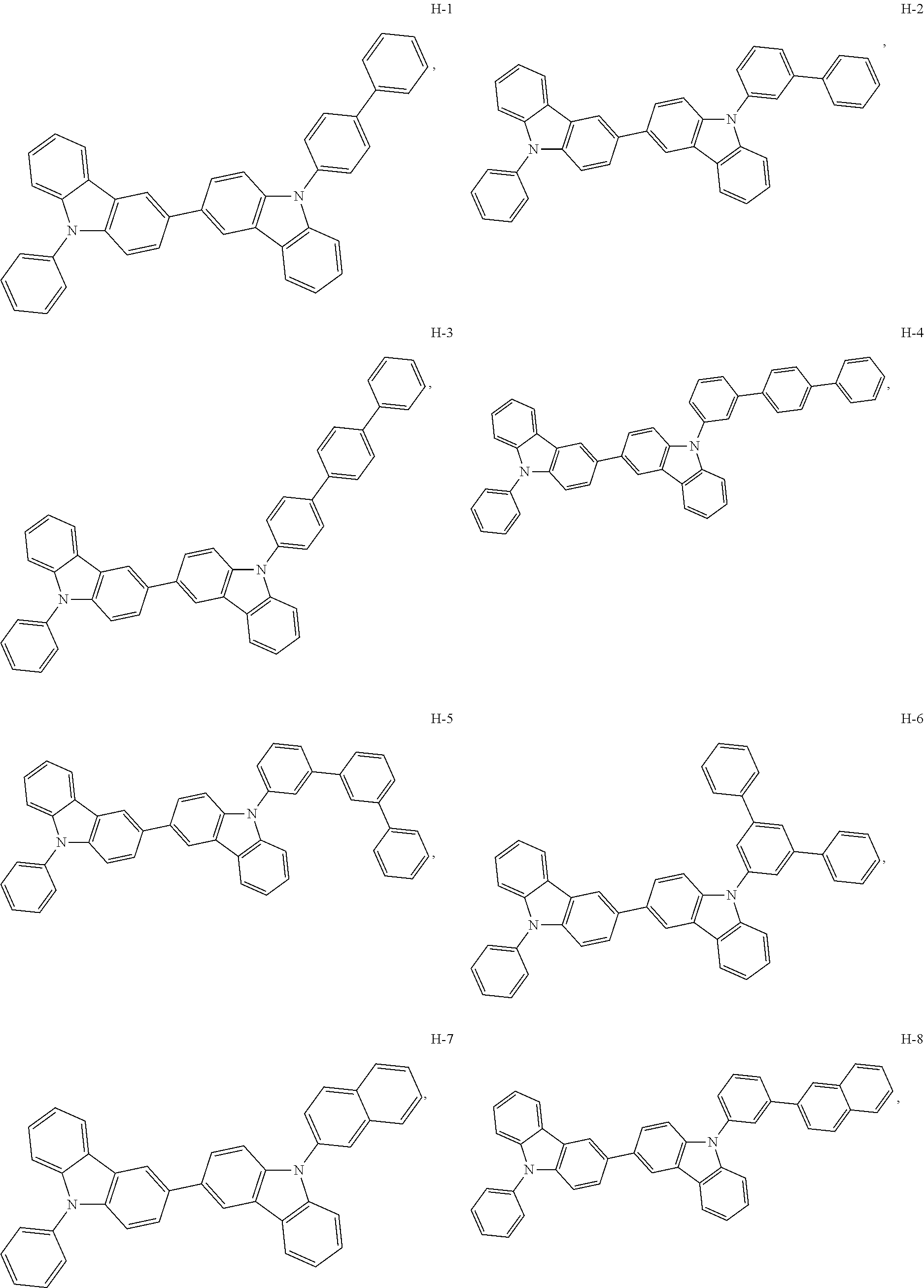

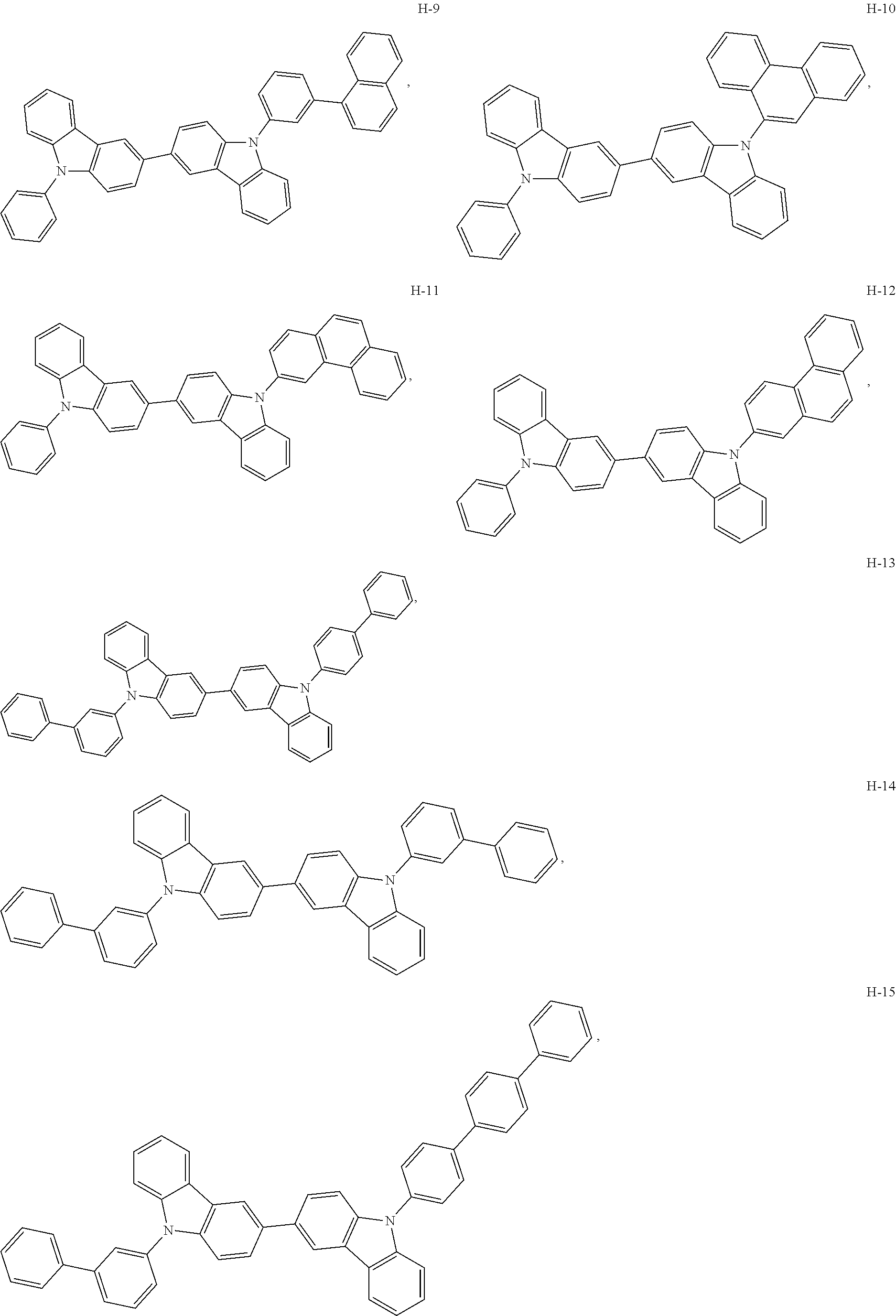

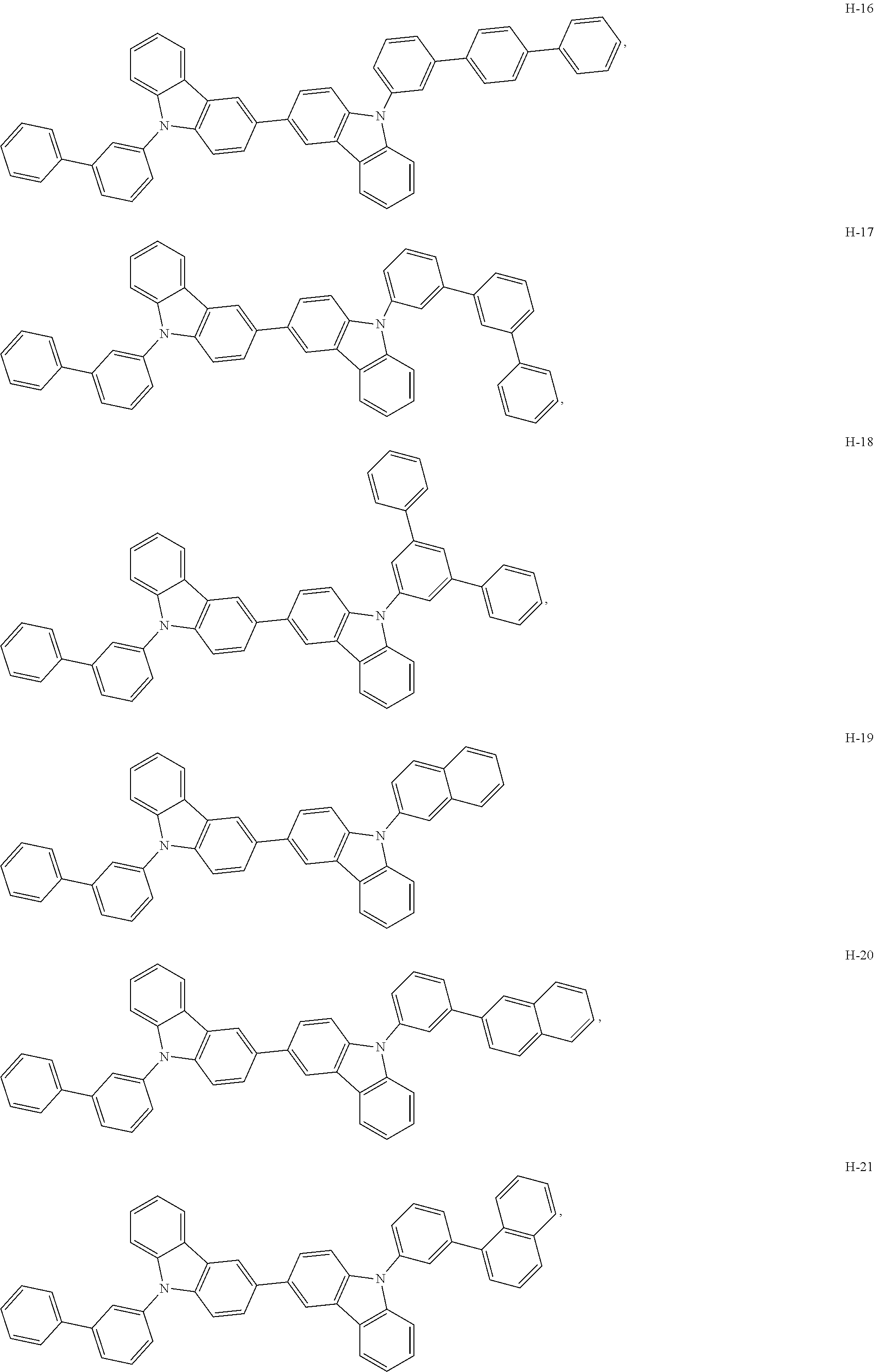

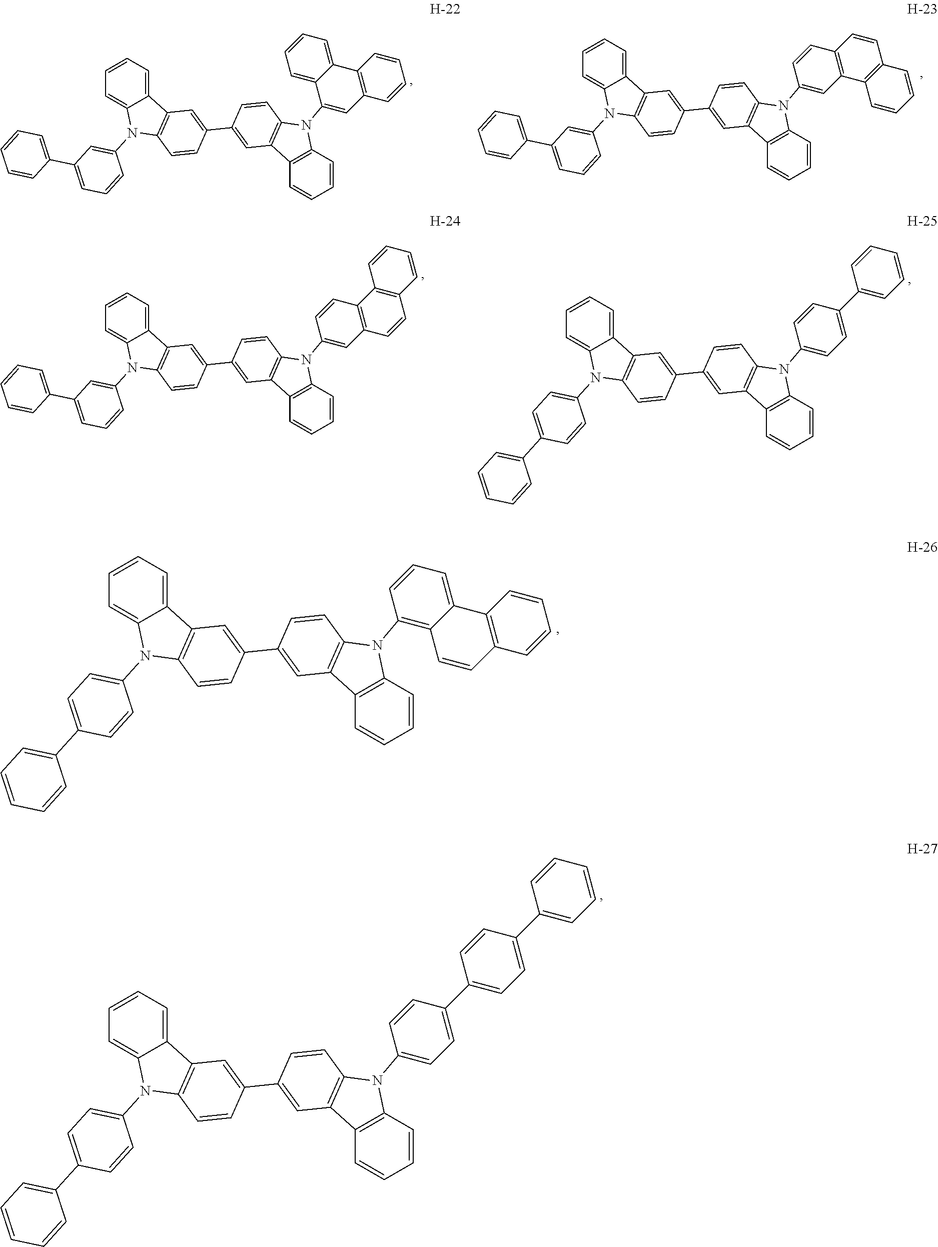

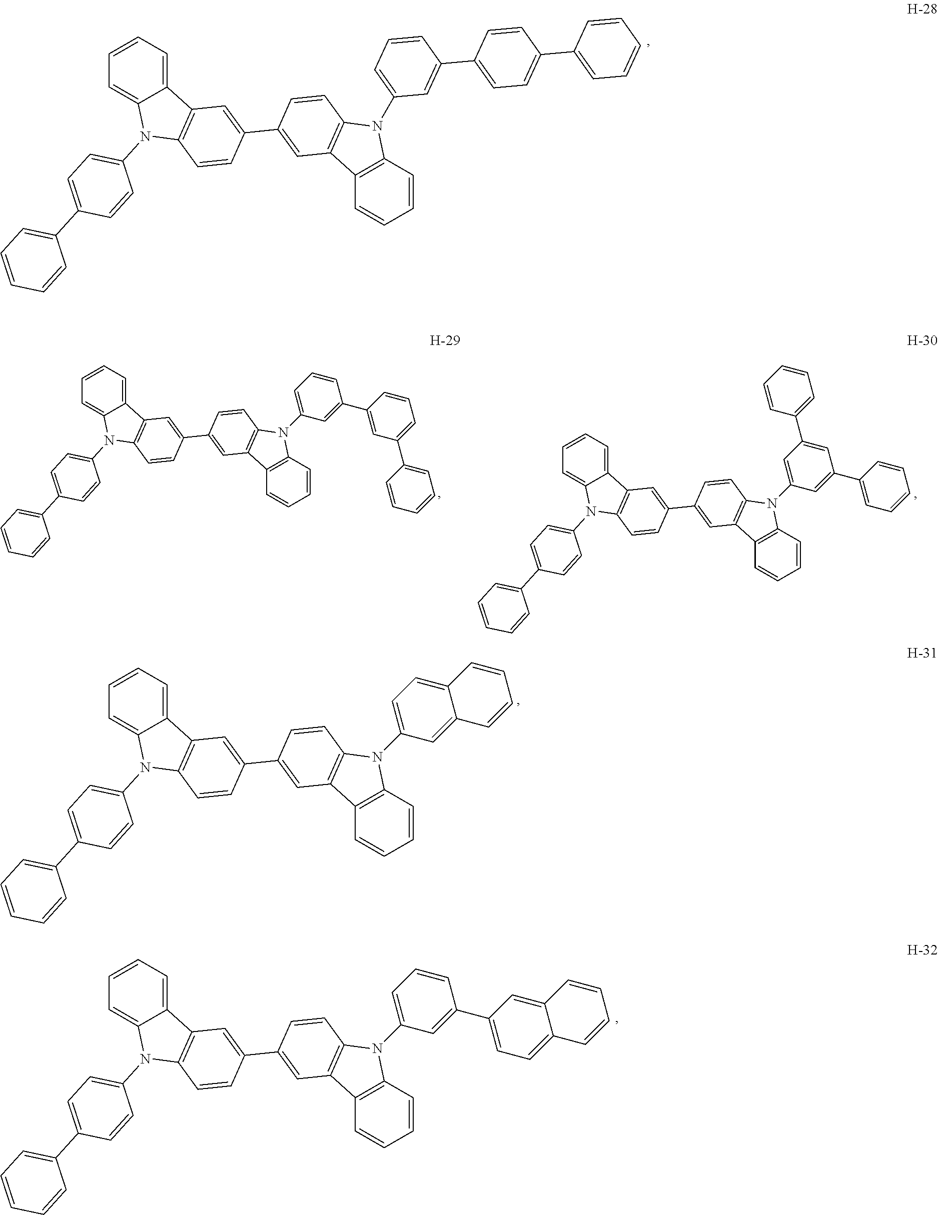

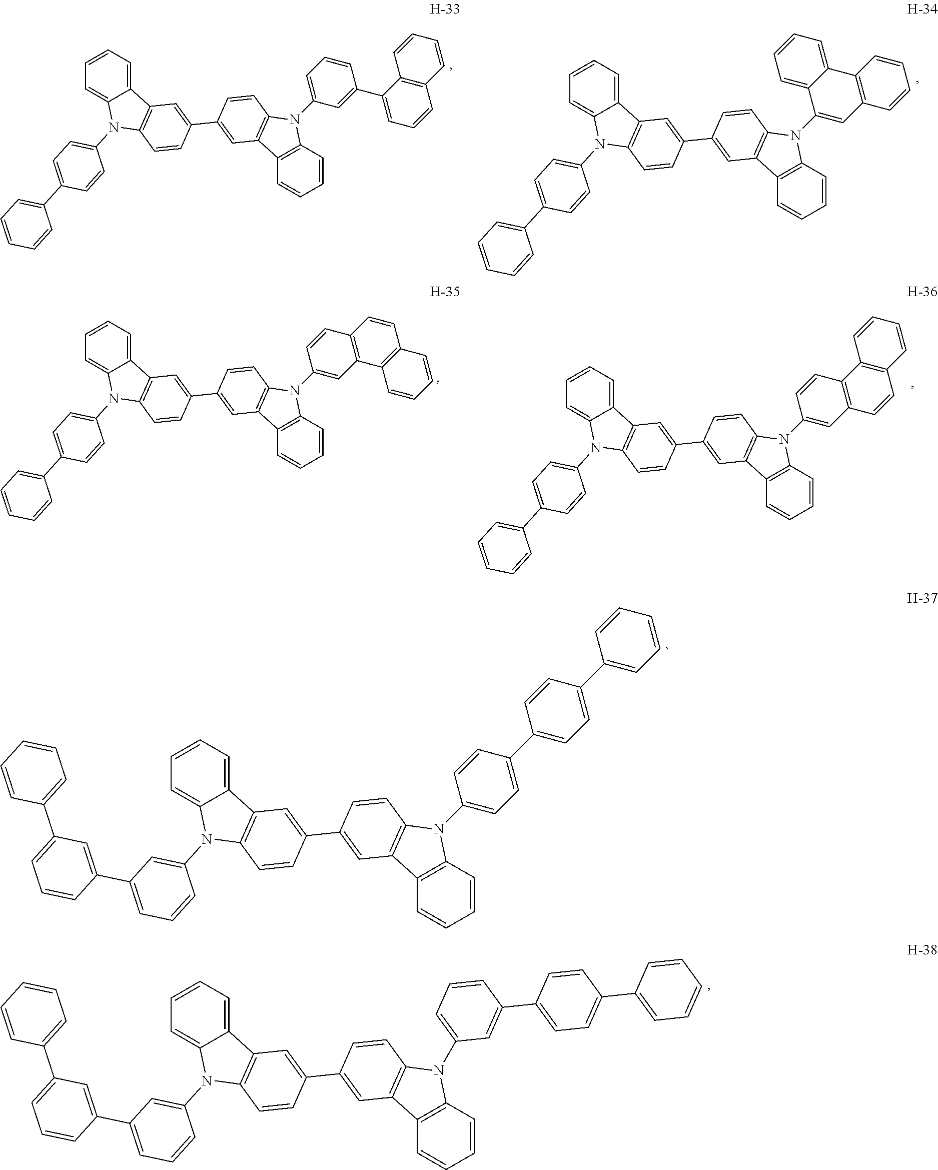

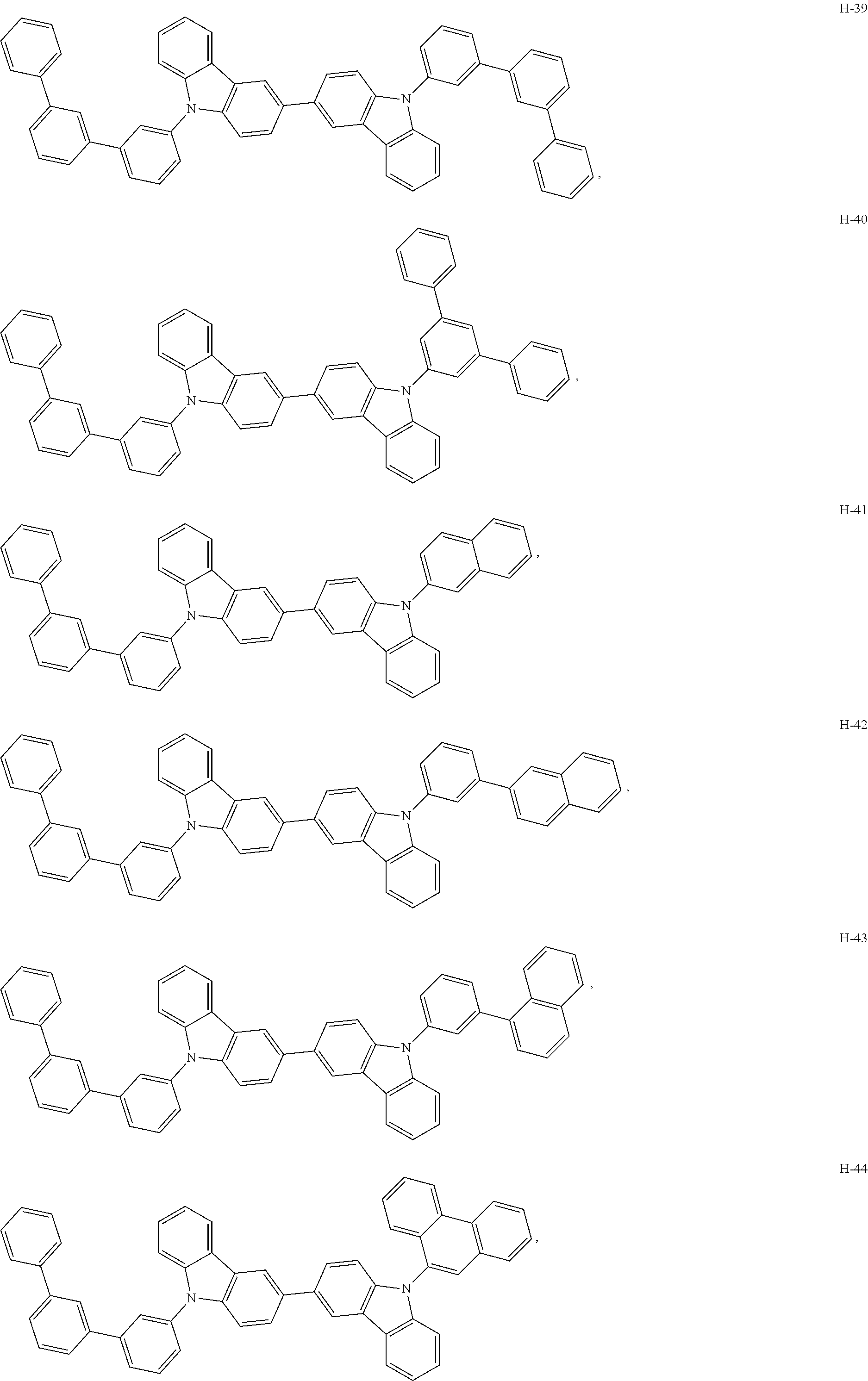

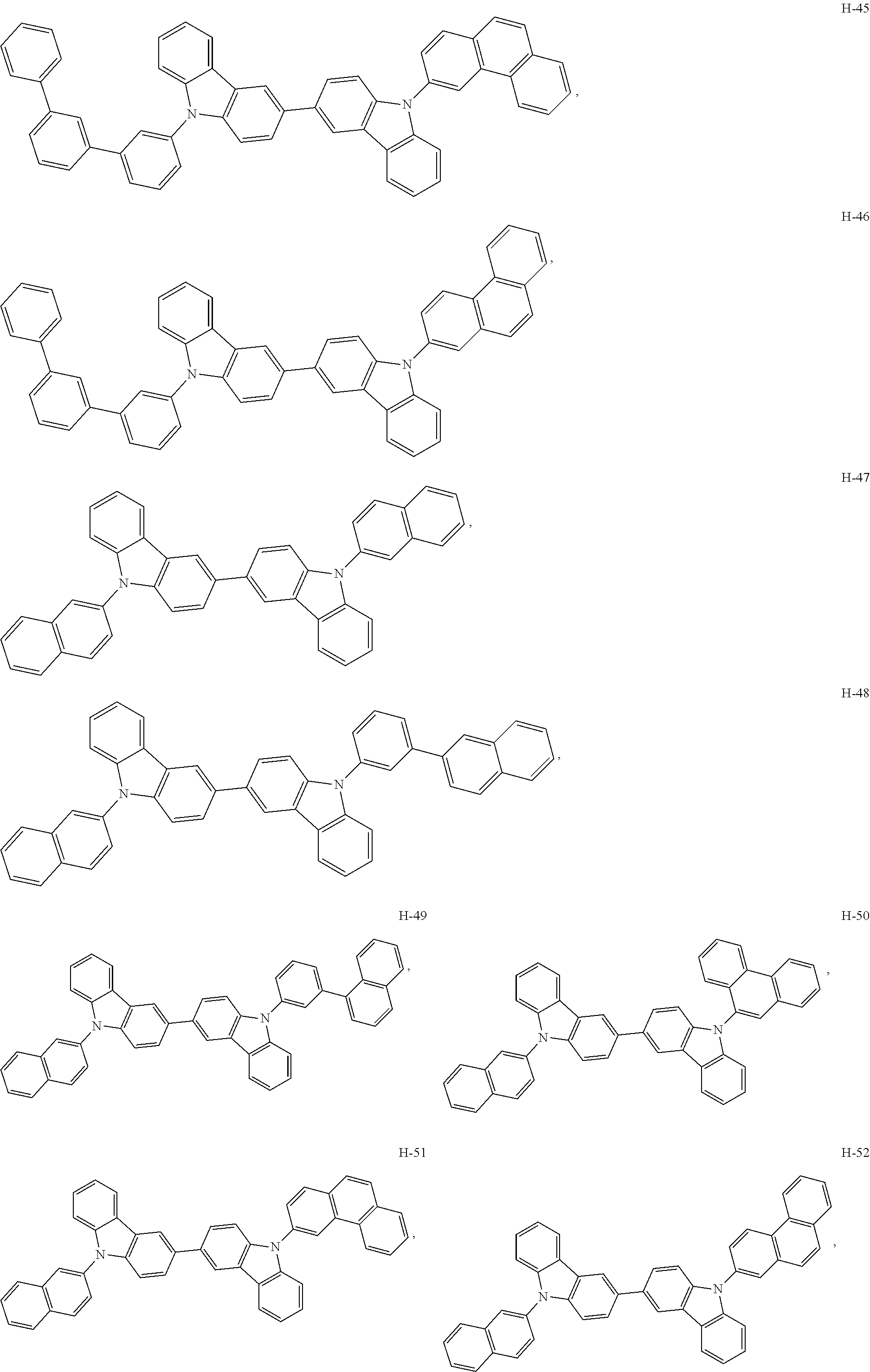

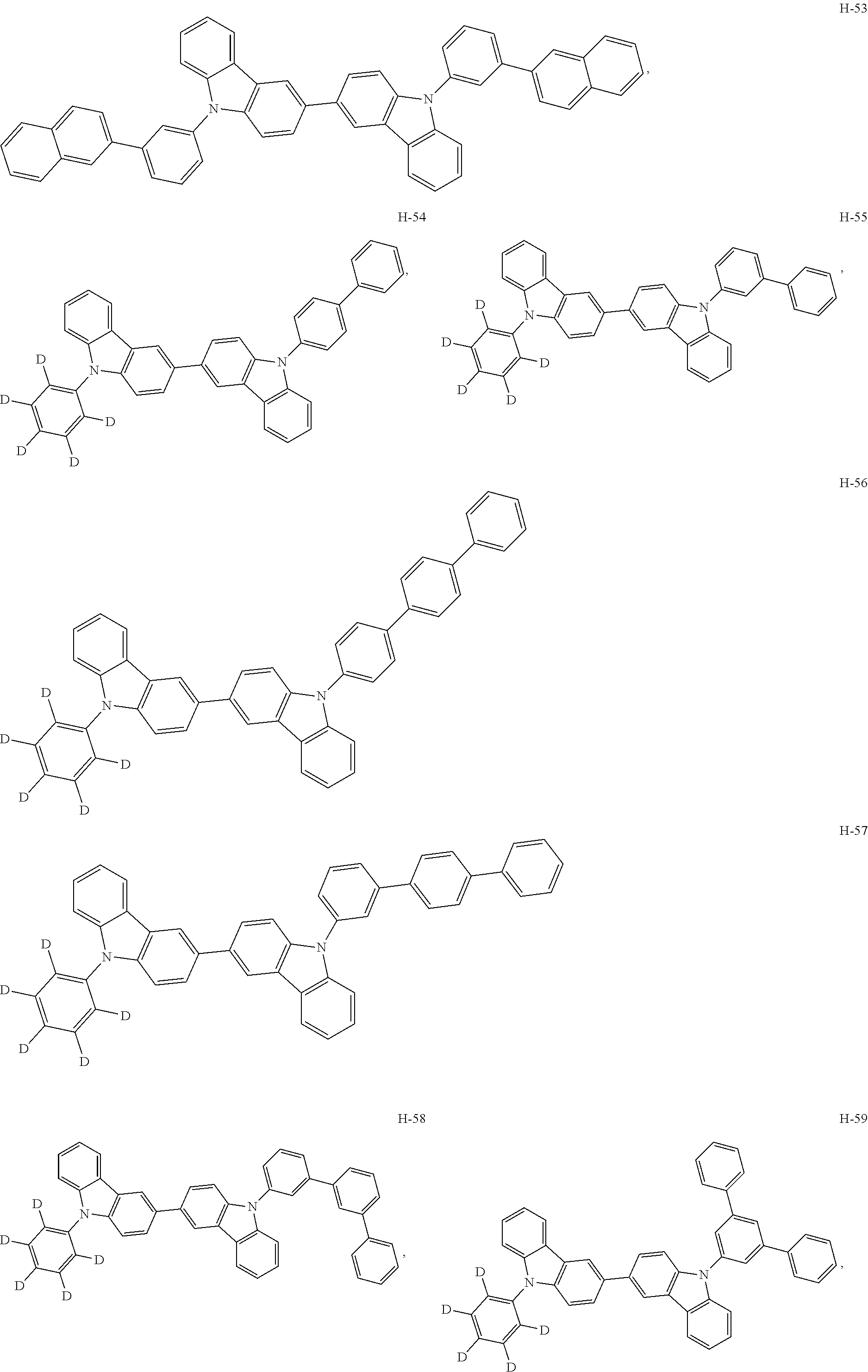

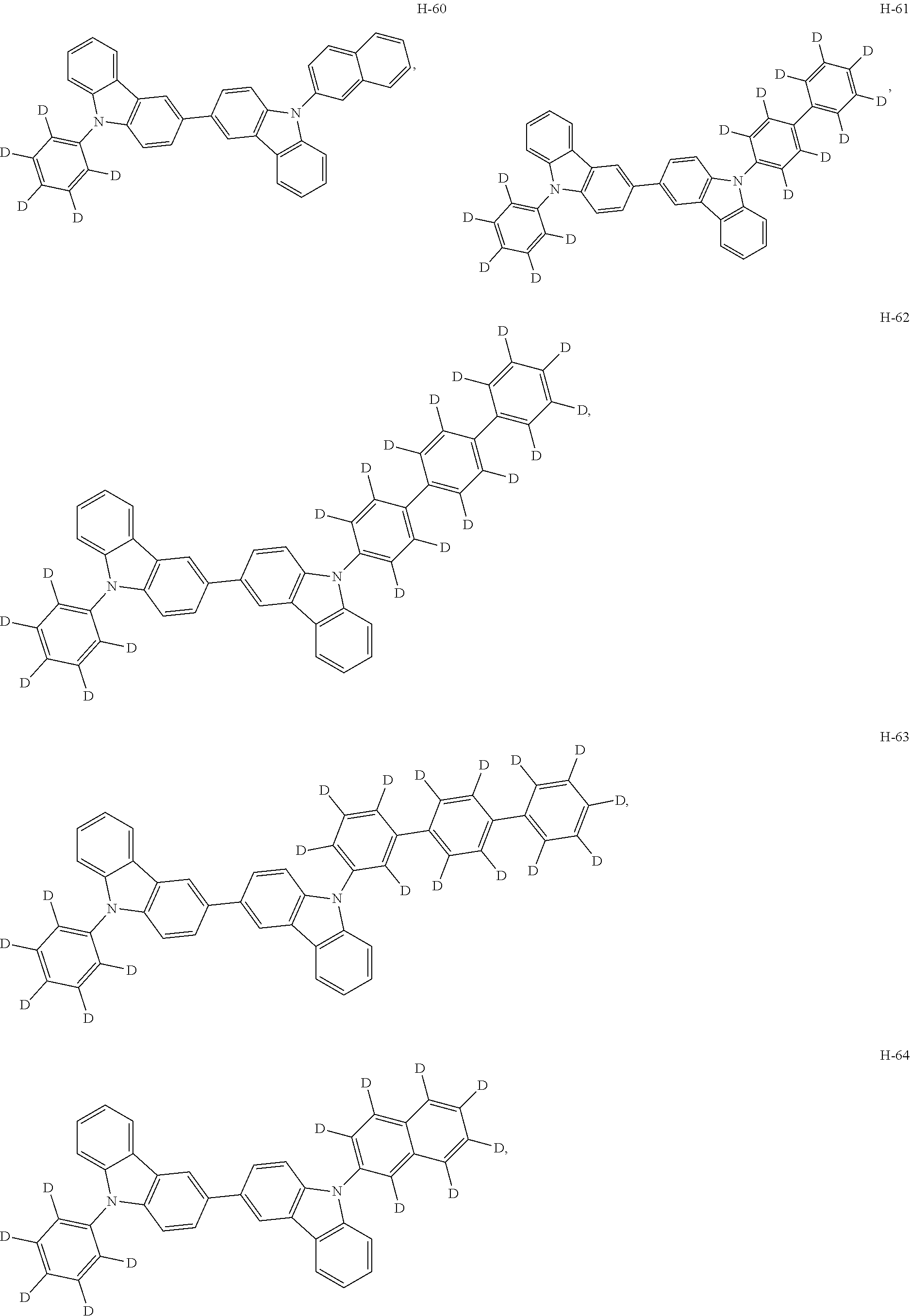

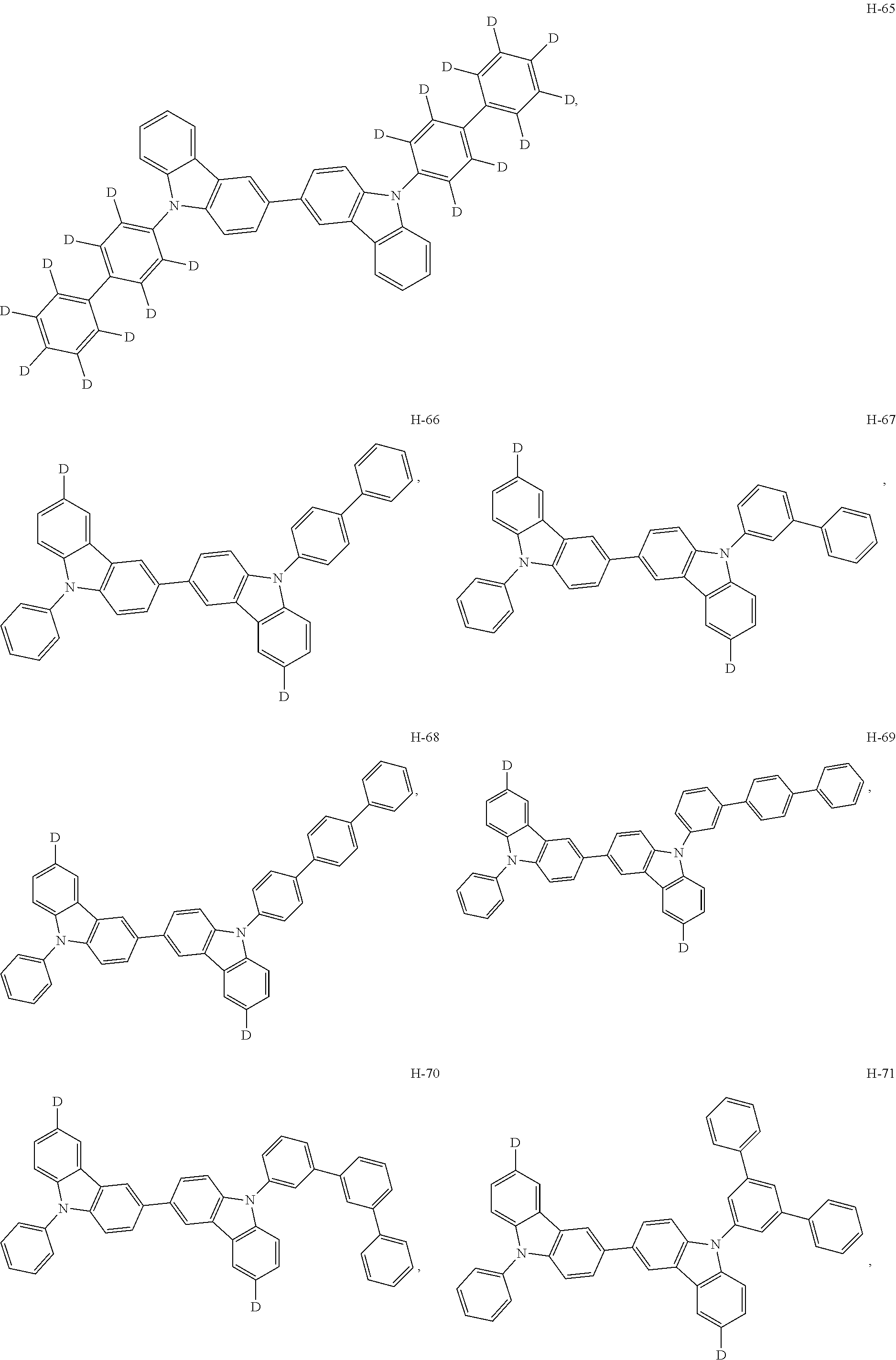

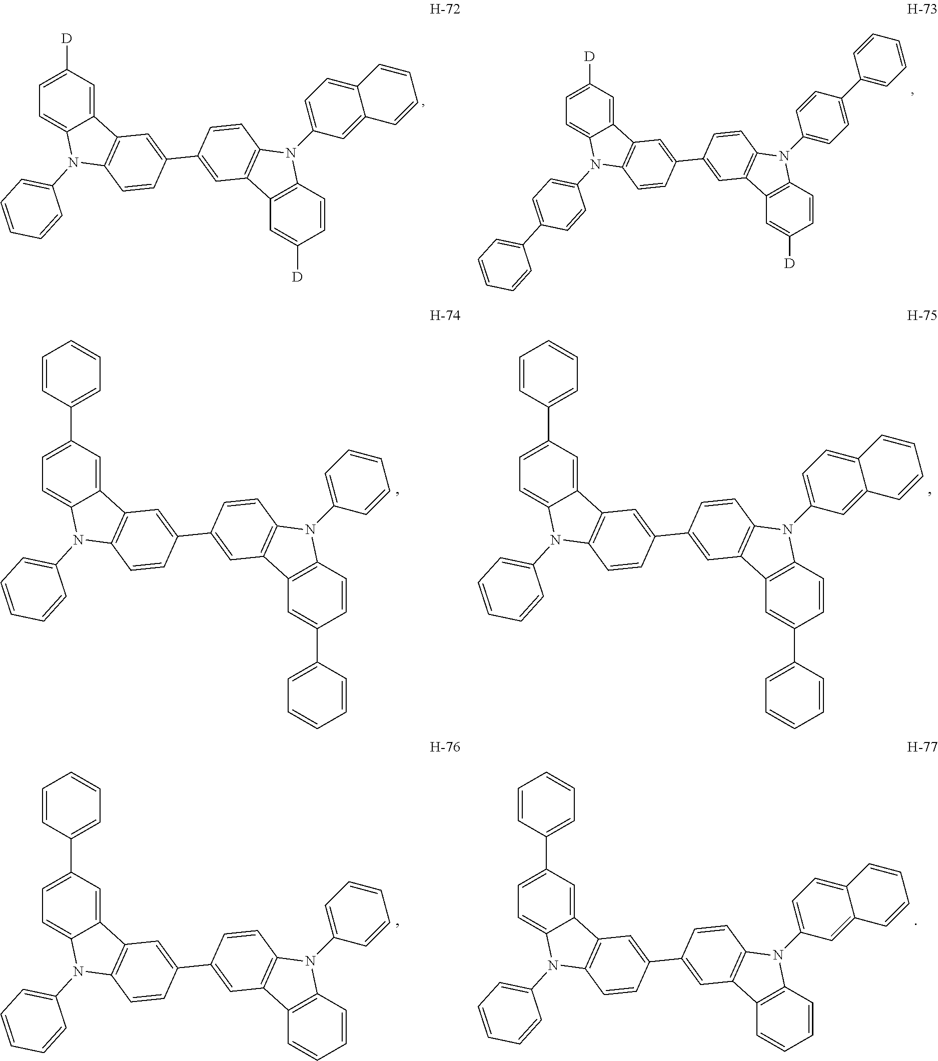

According to an embodiment of the present disclosure, wherein the second host compound is selected from the group consisting of:

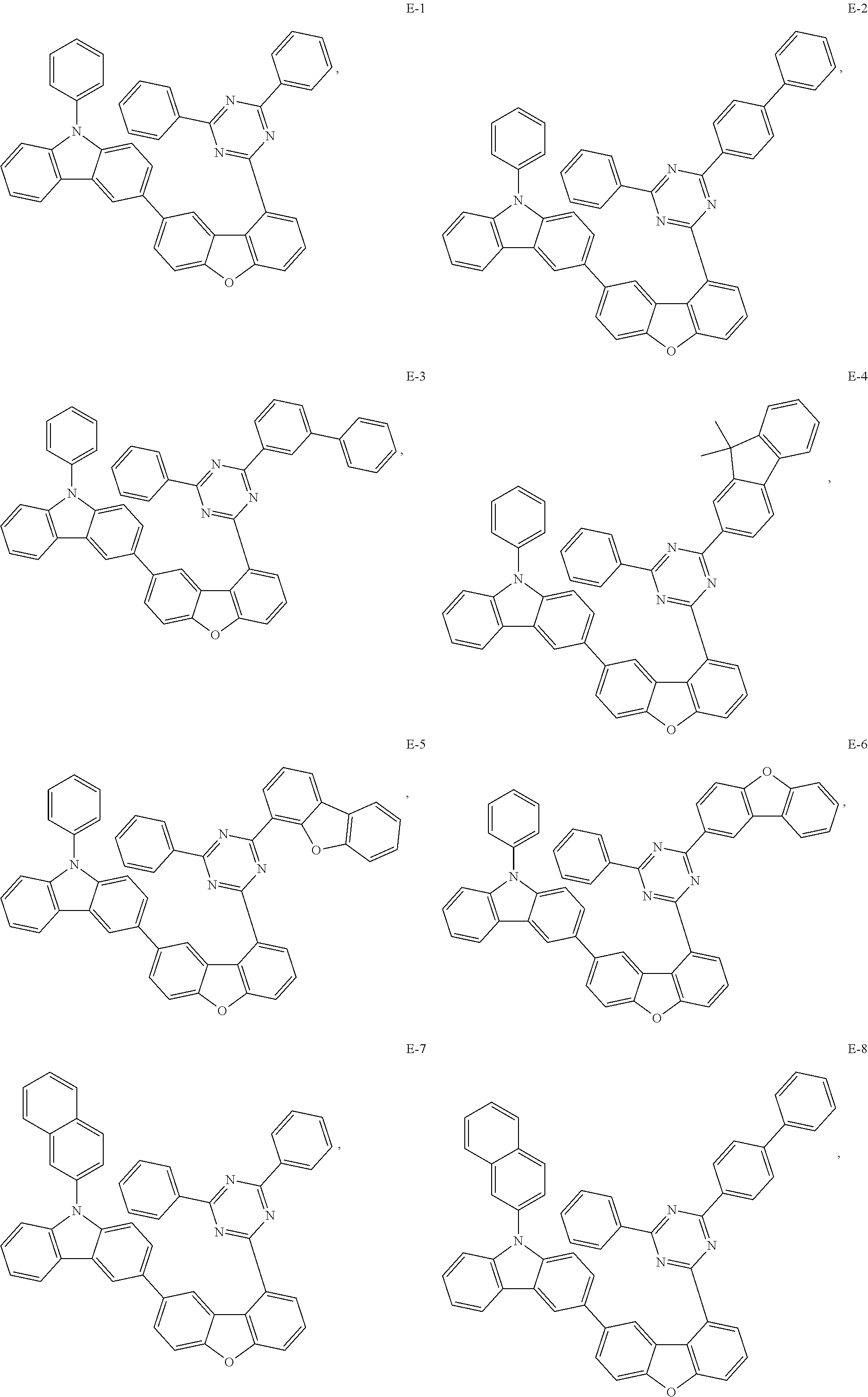

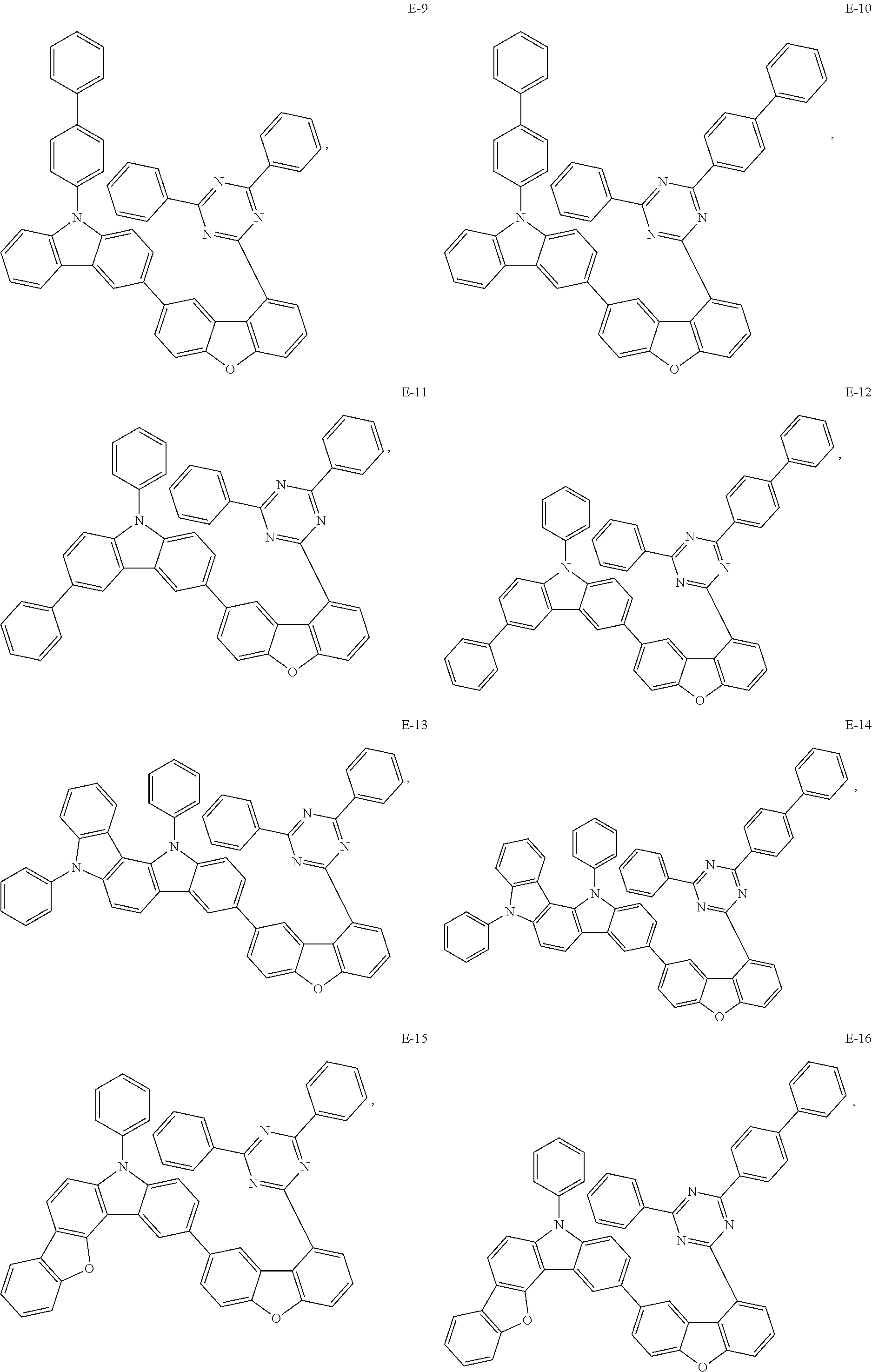

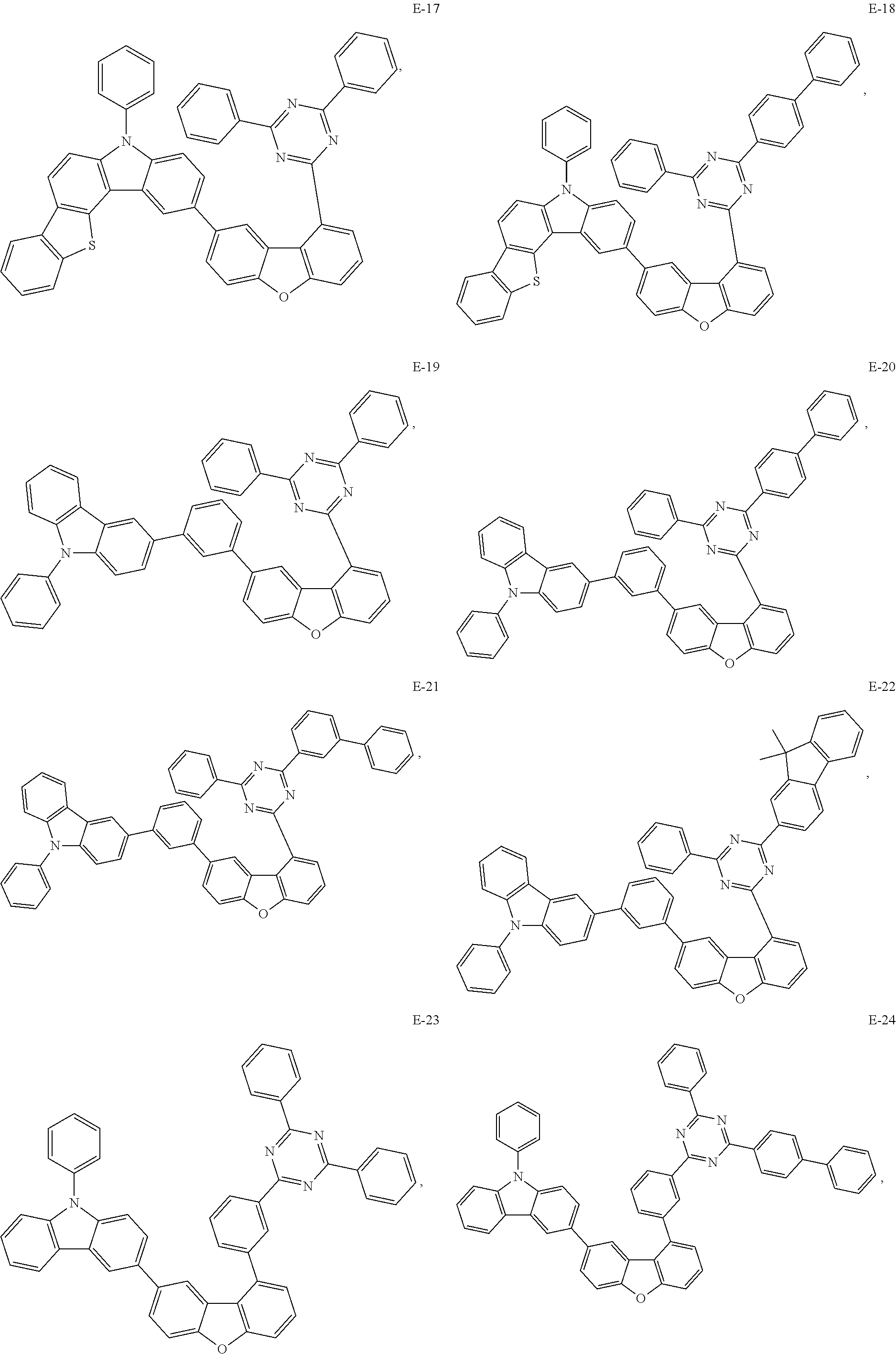

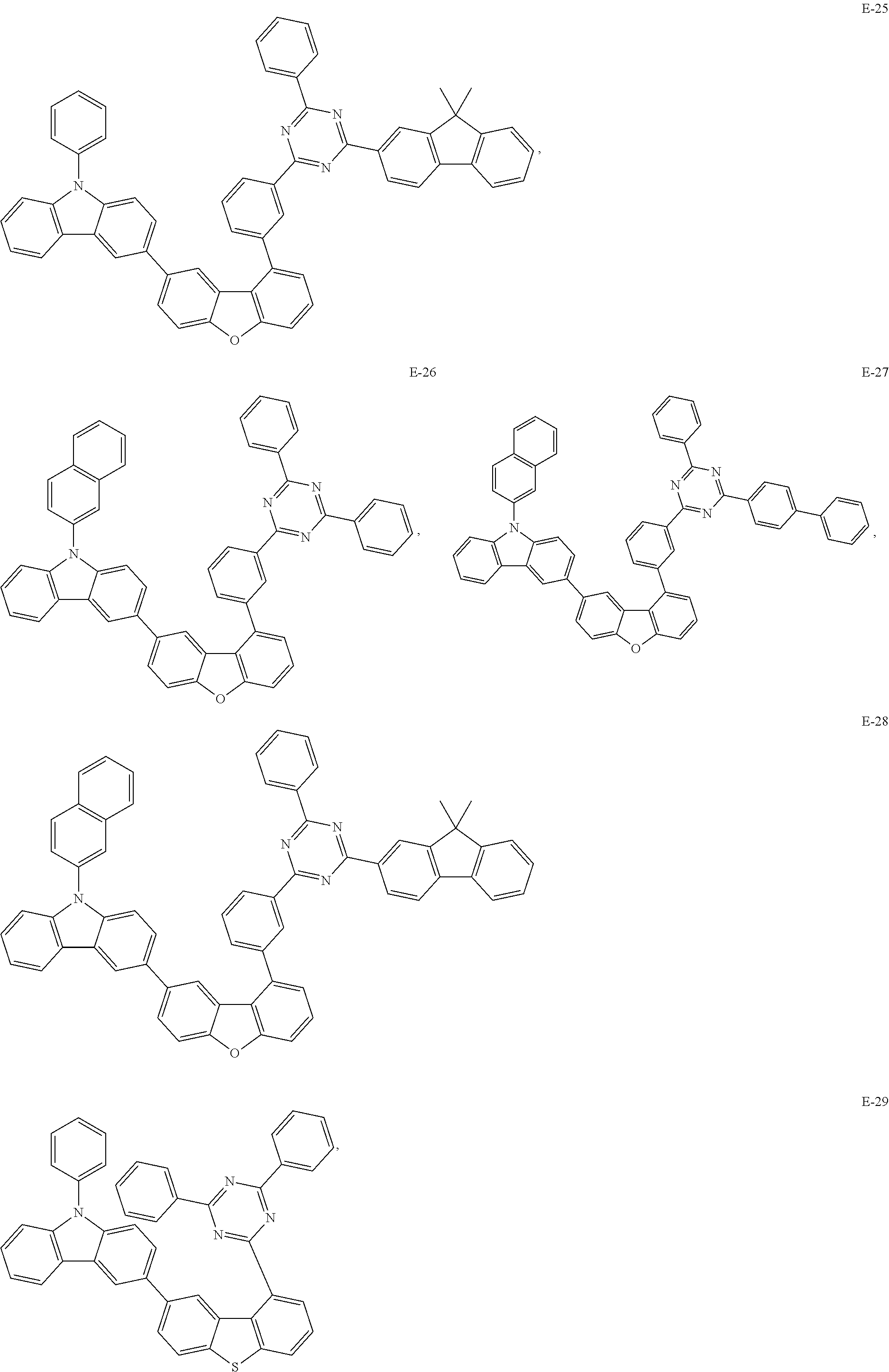

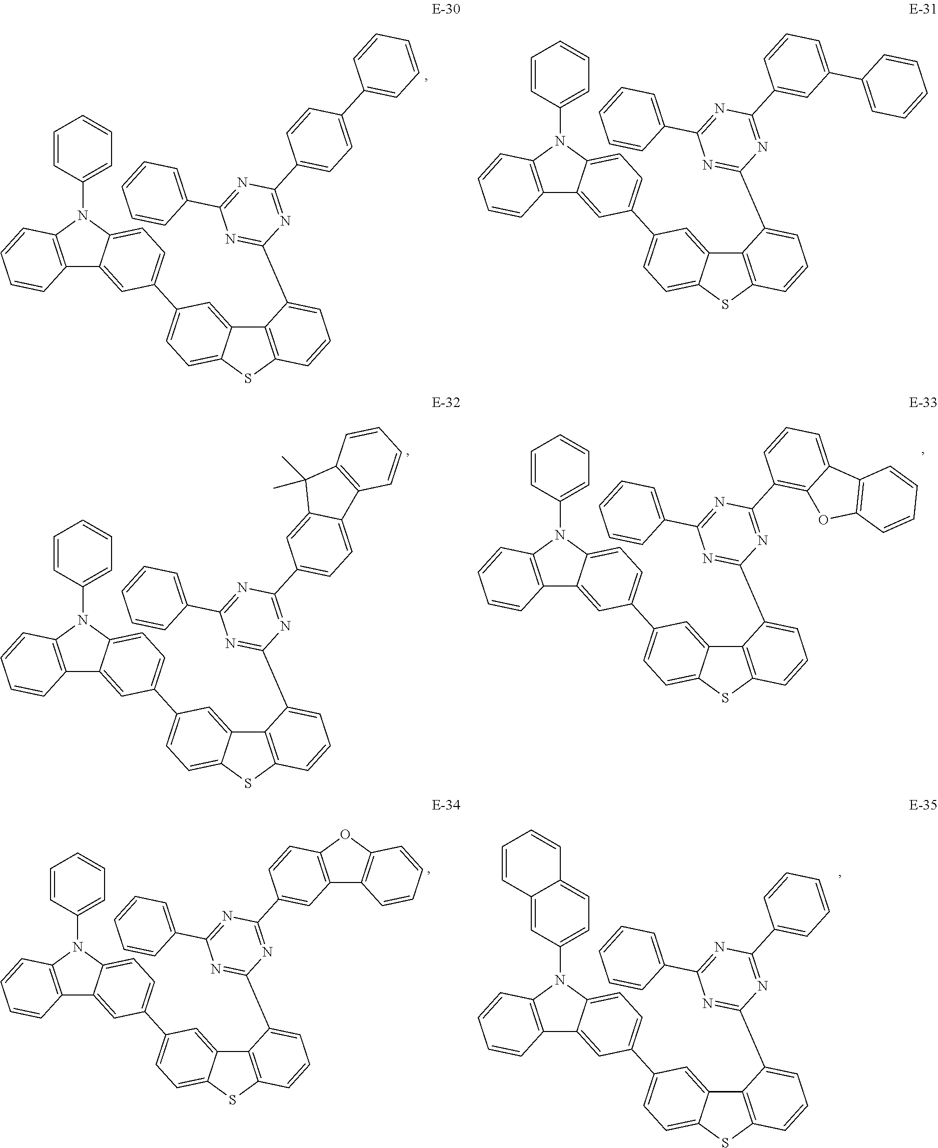

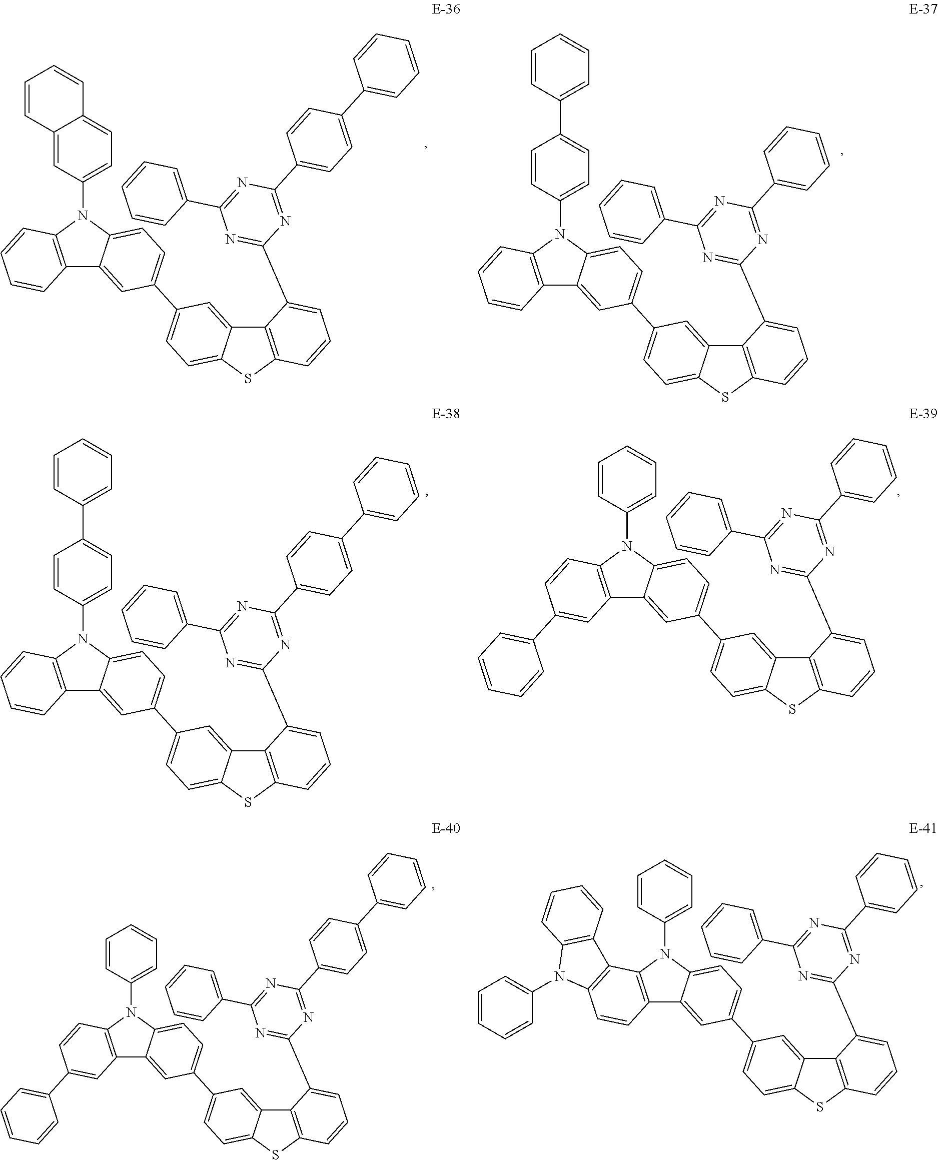

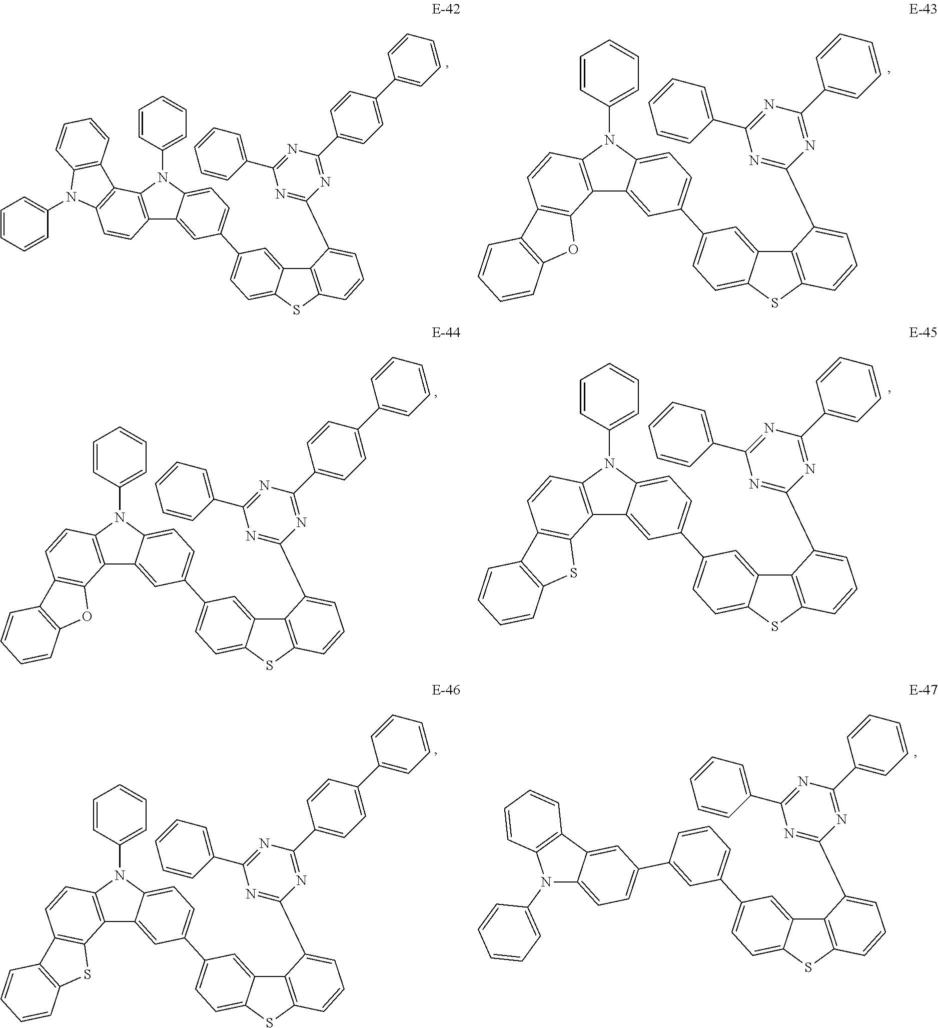

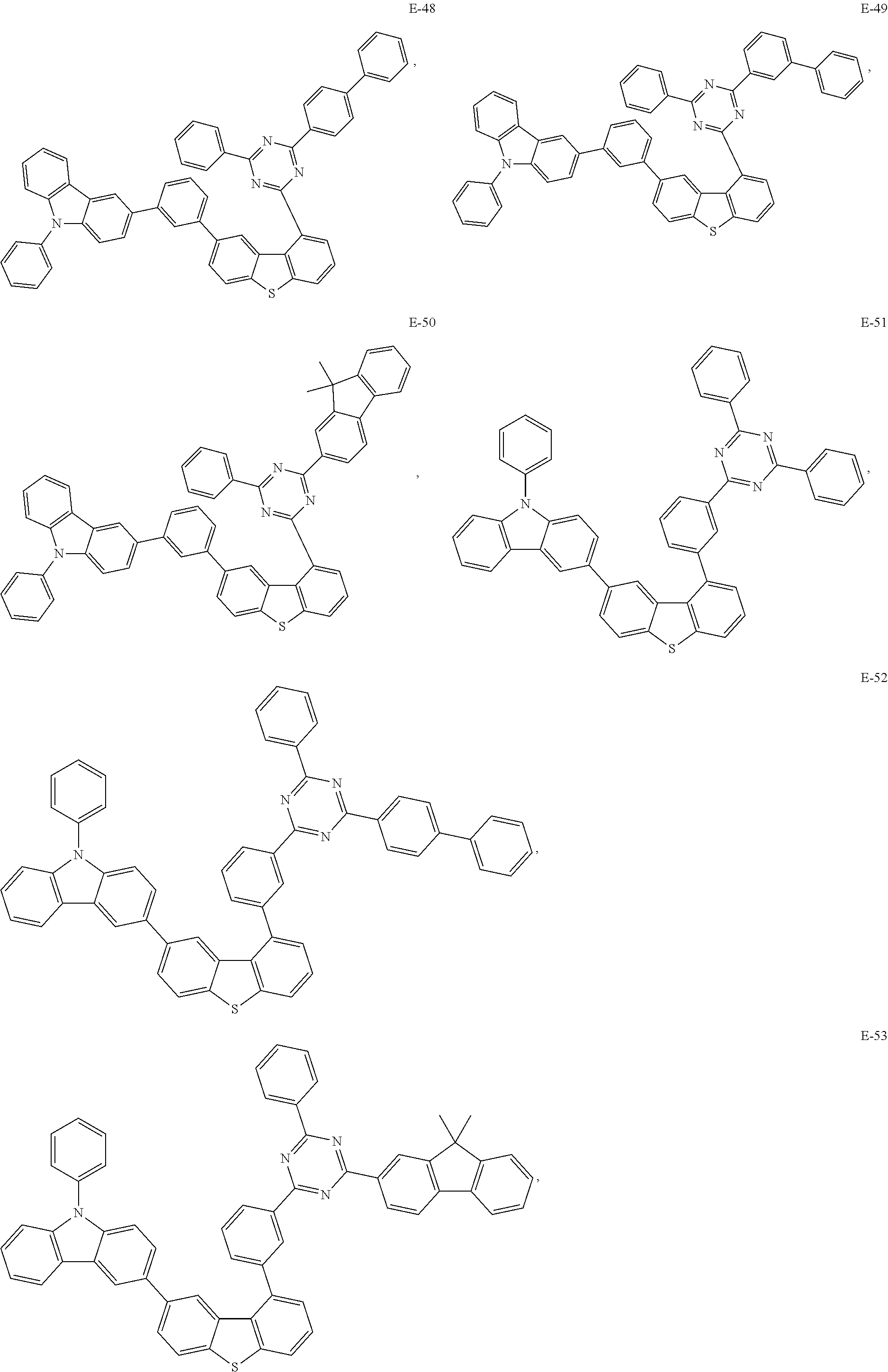

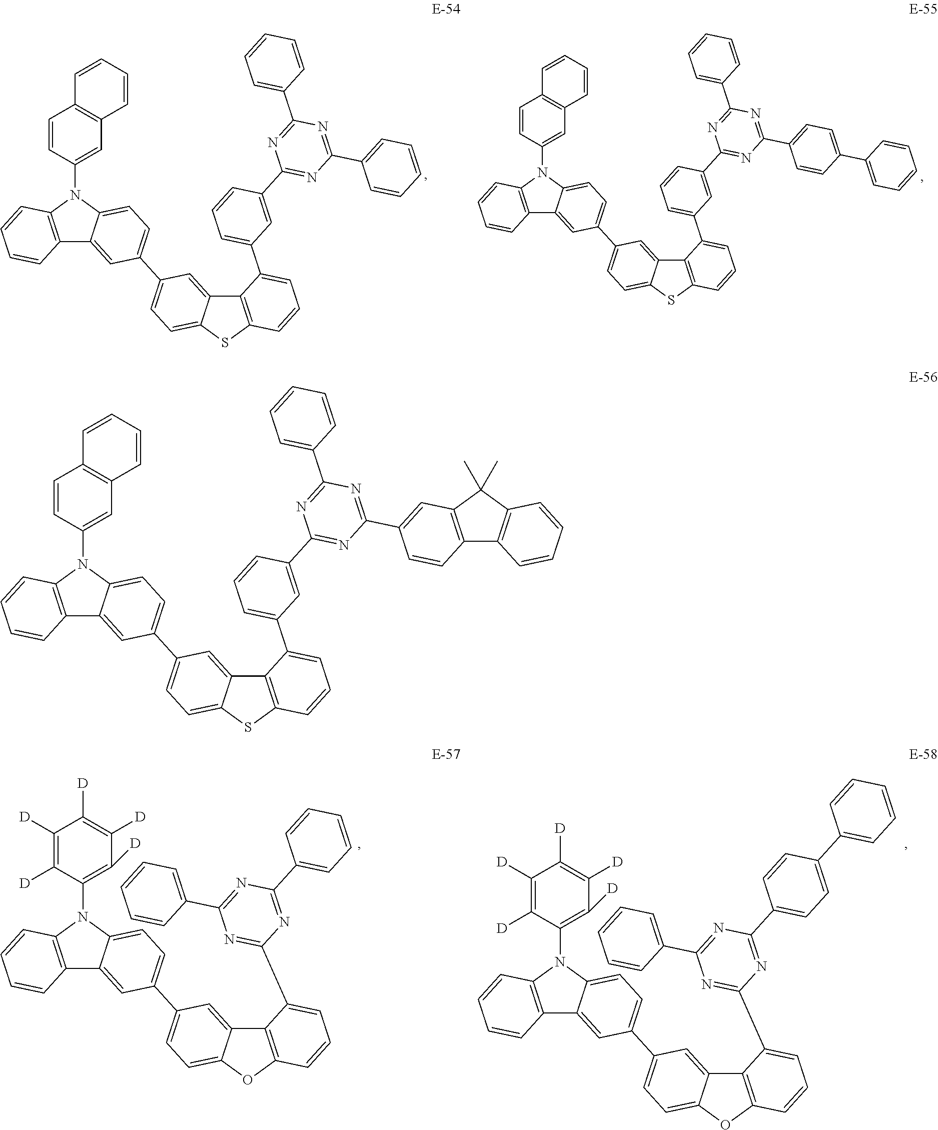

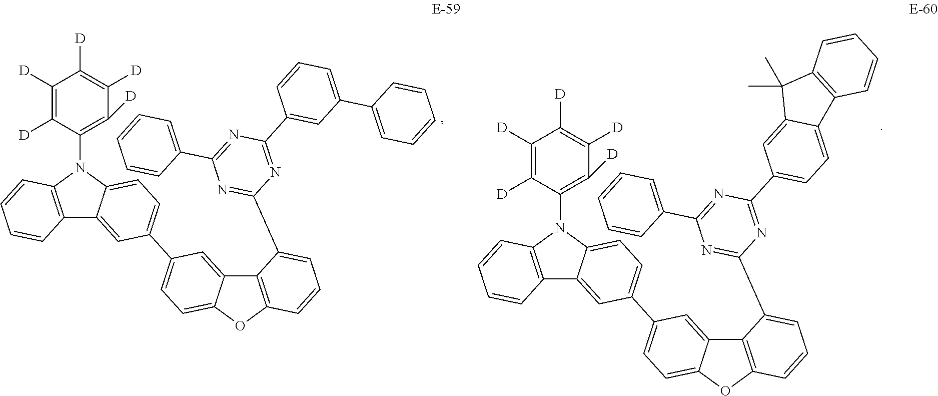 [SMILES: [2H]c1c([2H])c([2H])c(-n2c3ccccc3c3cc(-c4ccc5oc6cccc(-c7nc(-c8ccccc8)nc(-c8cccc(-c9ccccc9)c8)n7)c6c5c4)ccc32)c([2H])c1[2H] ; [2H]c1c([2H])c([2H])c(-n2c3ccccc3c3cc(-c4ccc5oc6cccc(-c7nc(-c8ccccc8)nc(-c8ccc9c(c8)C(C)(C)c8ccccc8-9)n7)c6c5c4)ccc32)c([2H])c1[2H]]

According to an embodiment of the present disclosure, wherein the dopant compound is selected from the group consisting of:

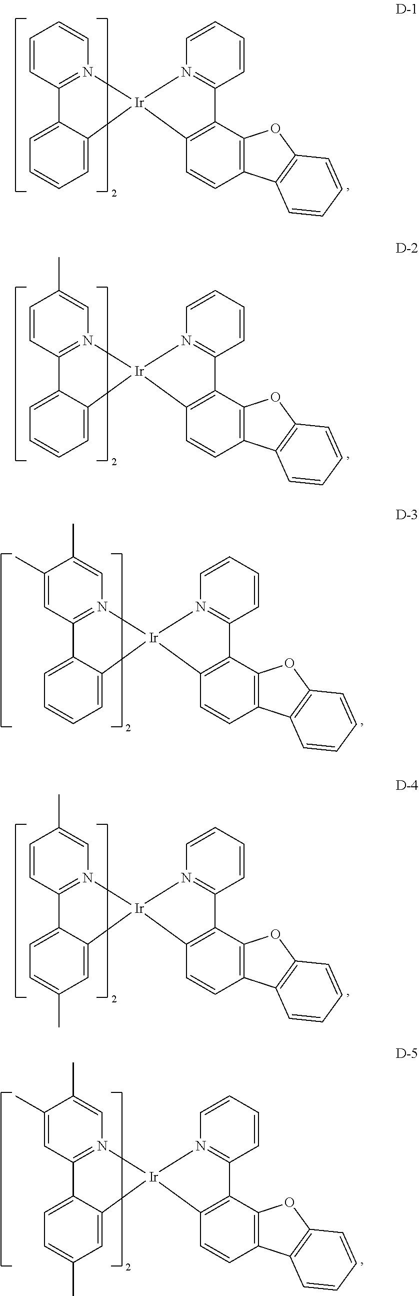

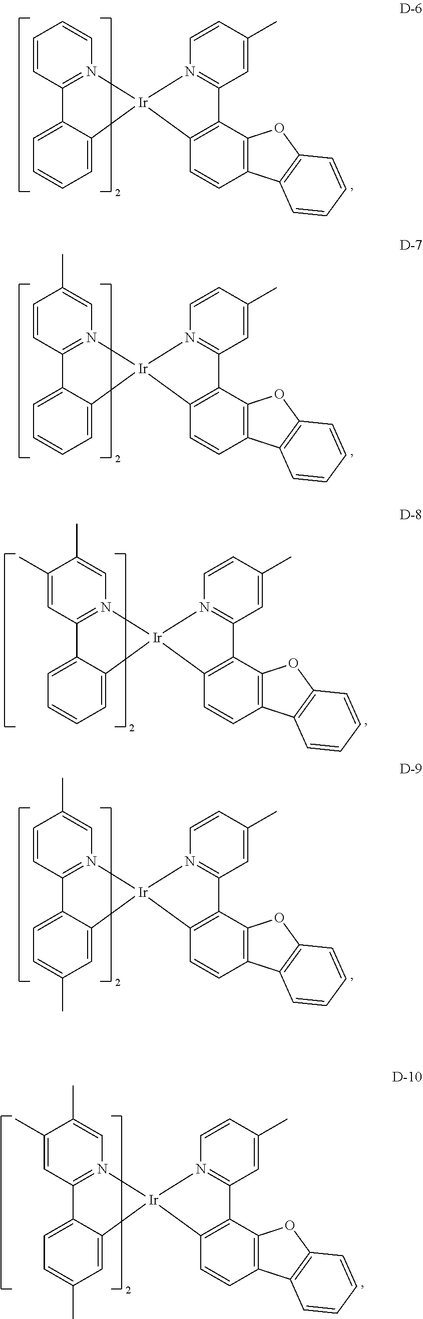

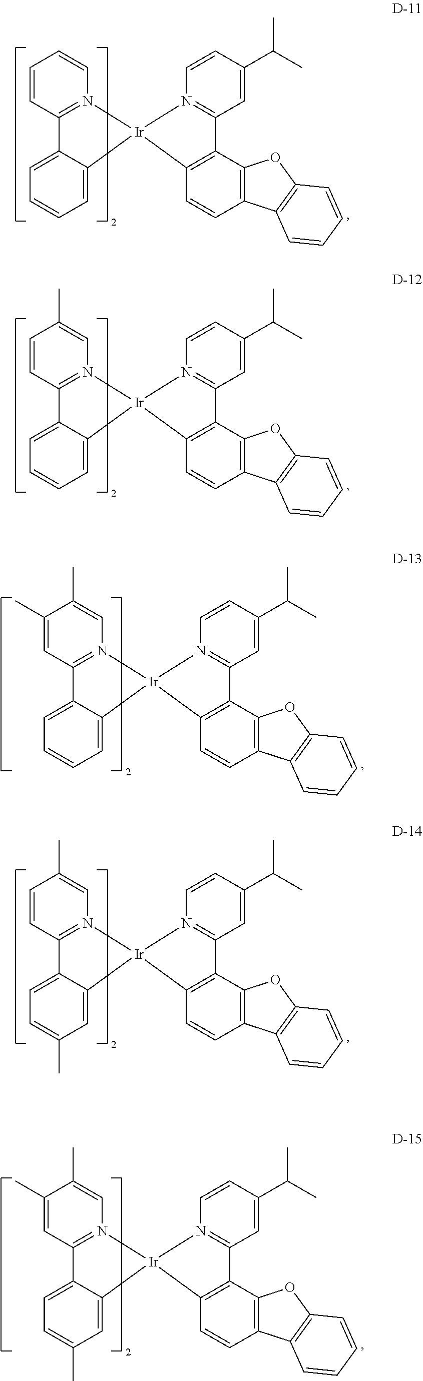

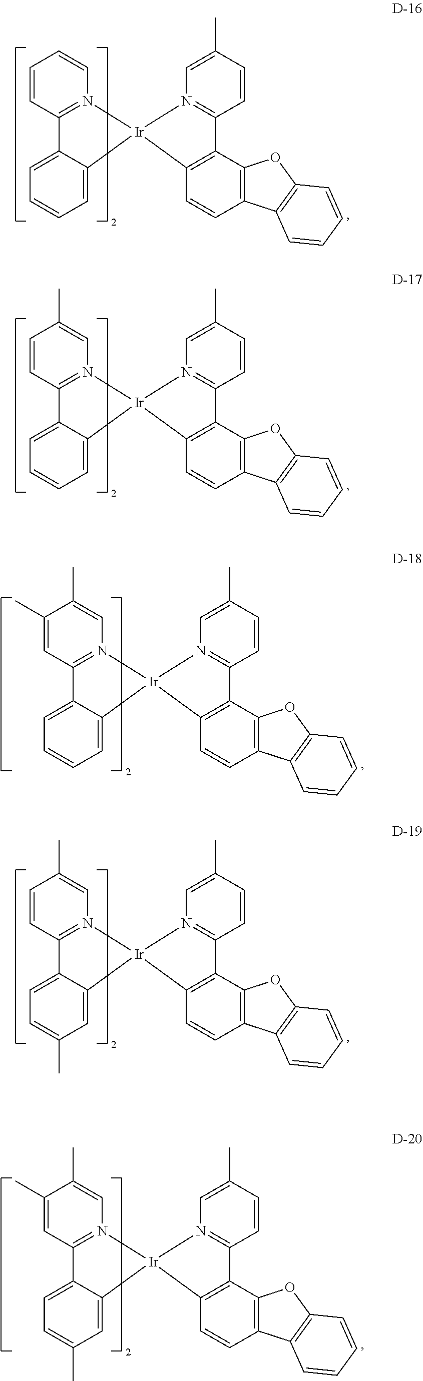

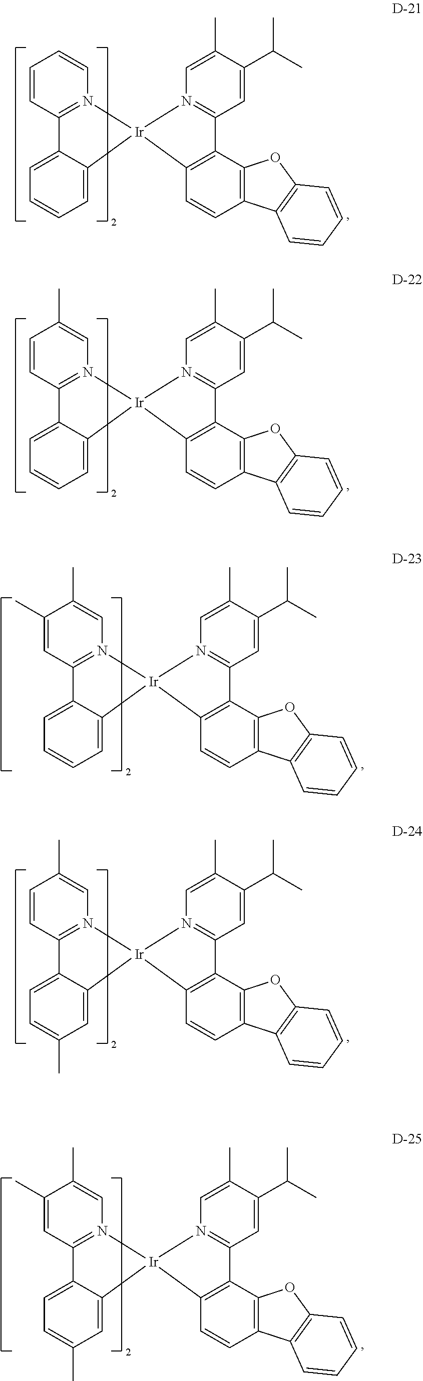

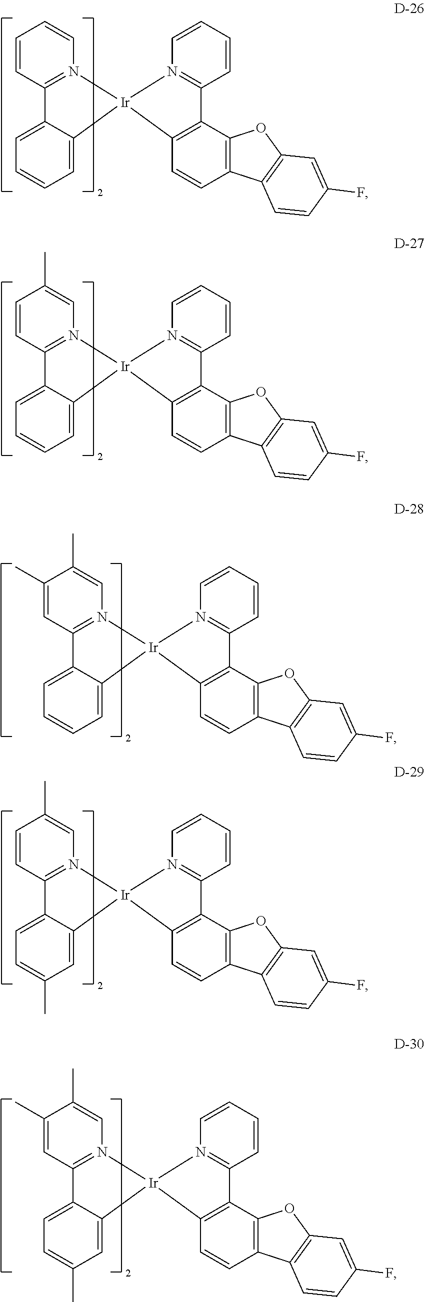

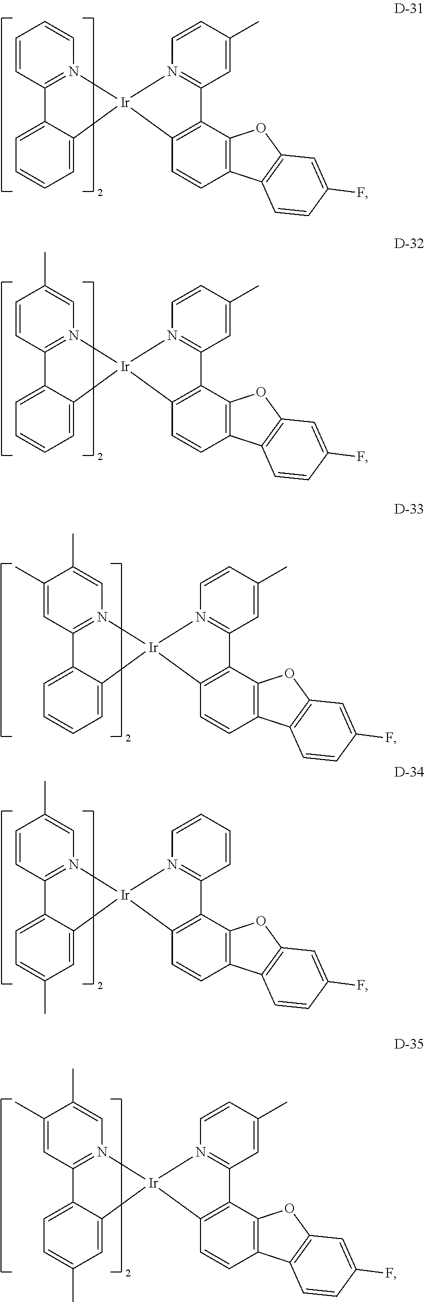

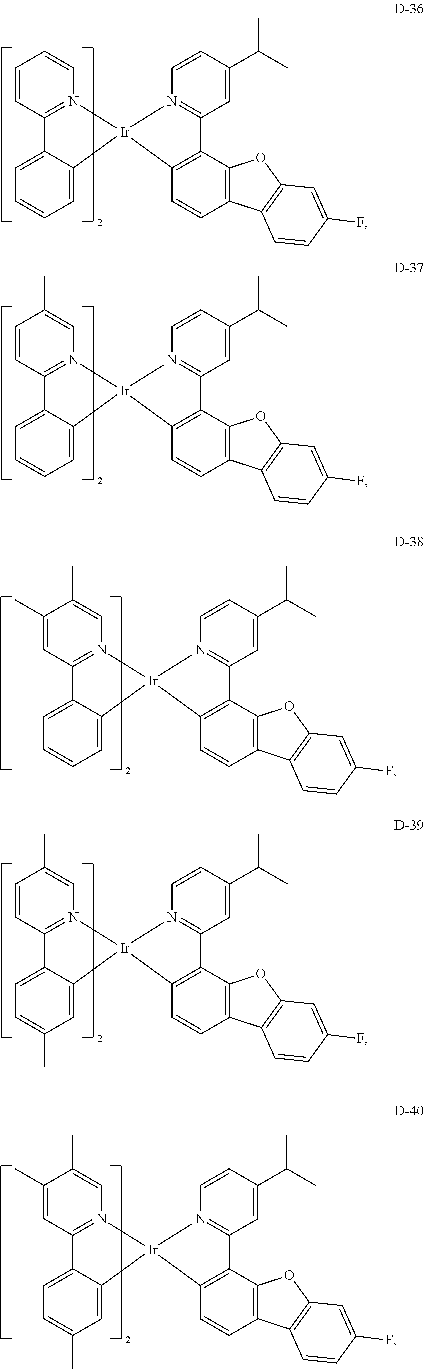

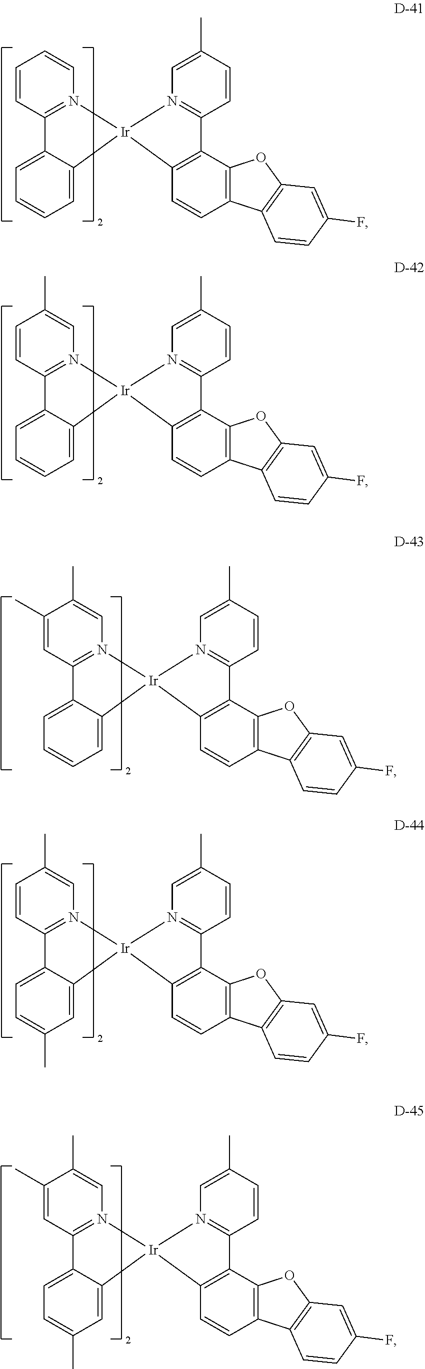

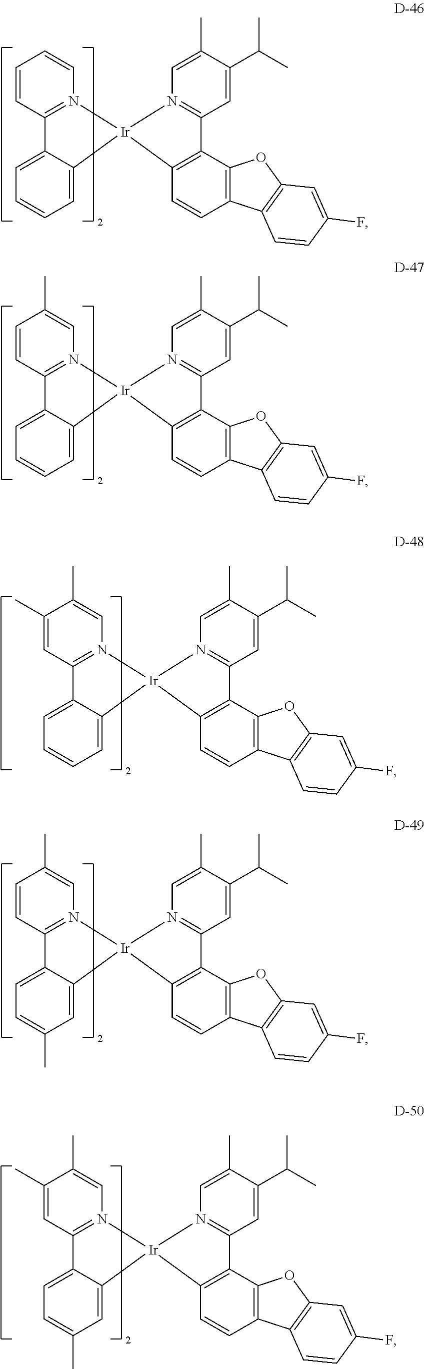

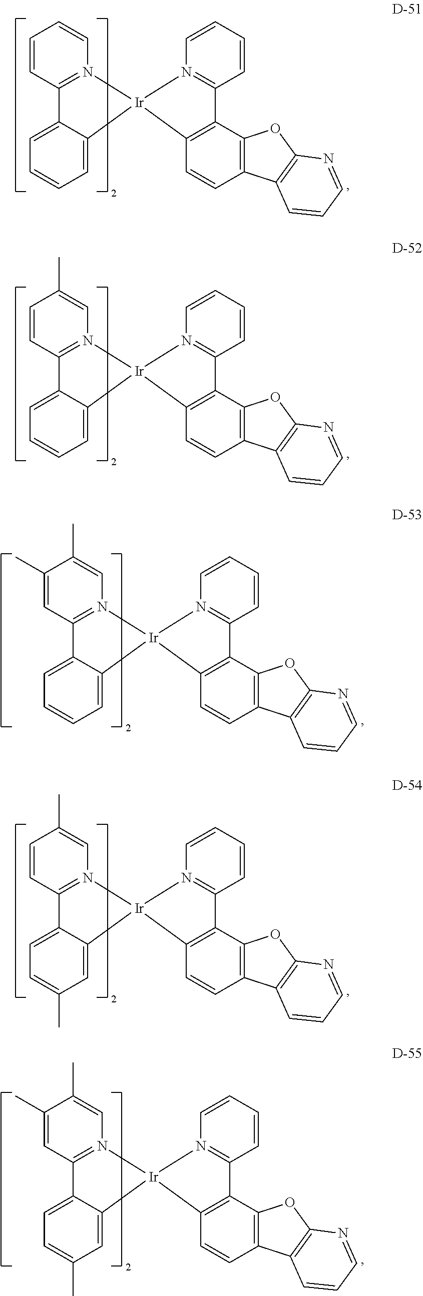

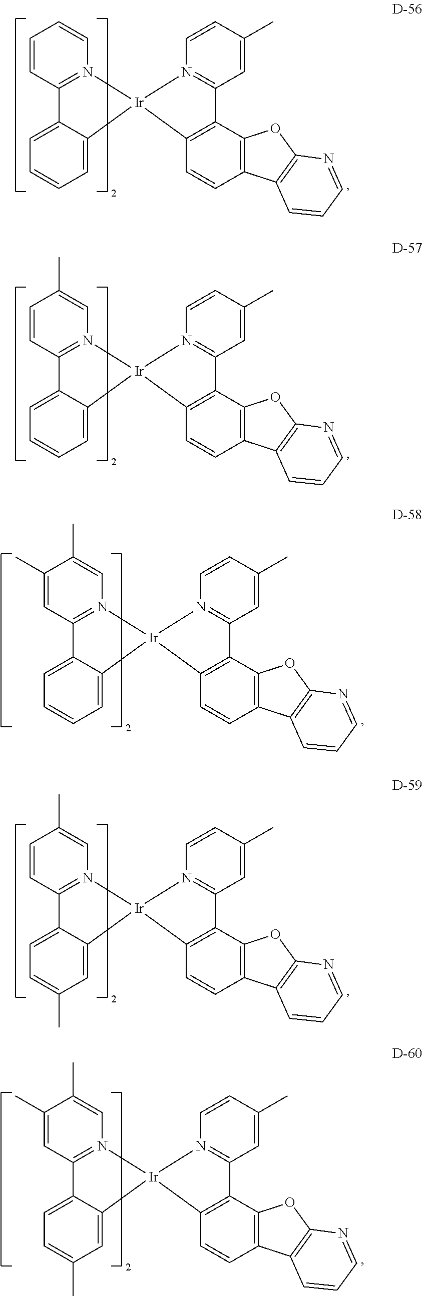

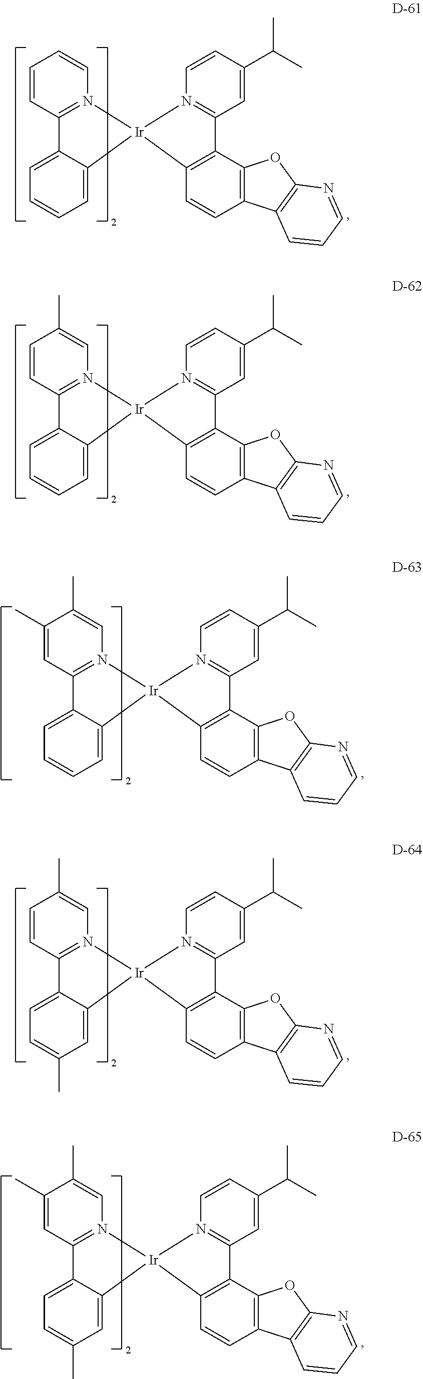

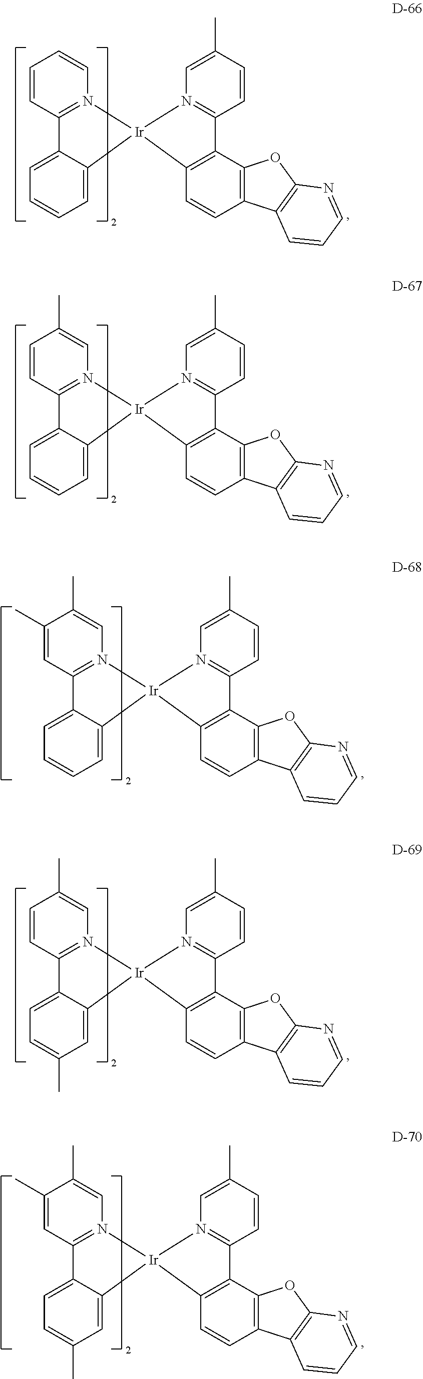

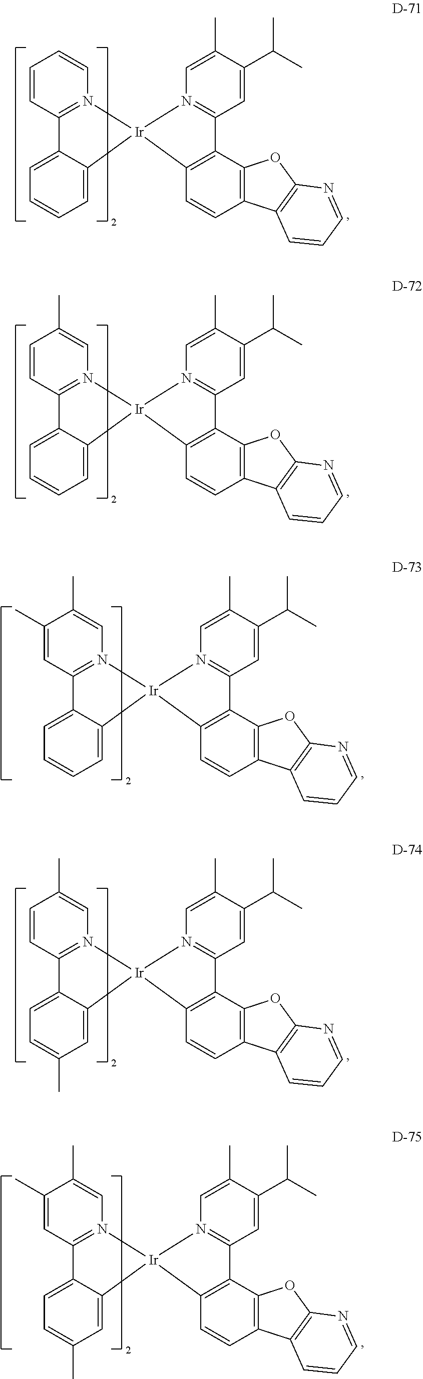

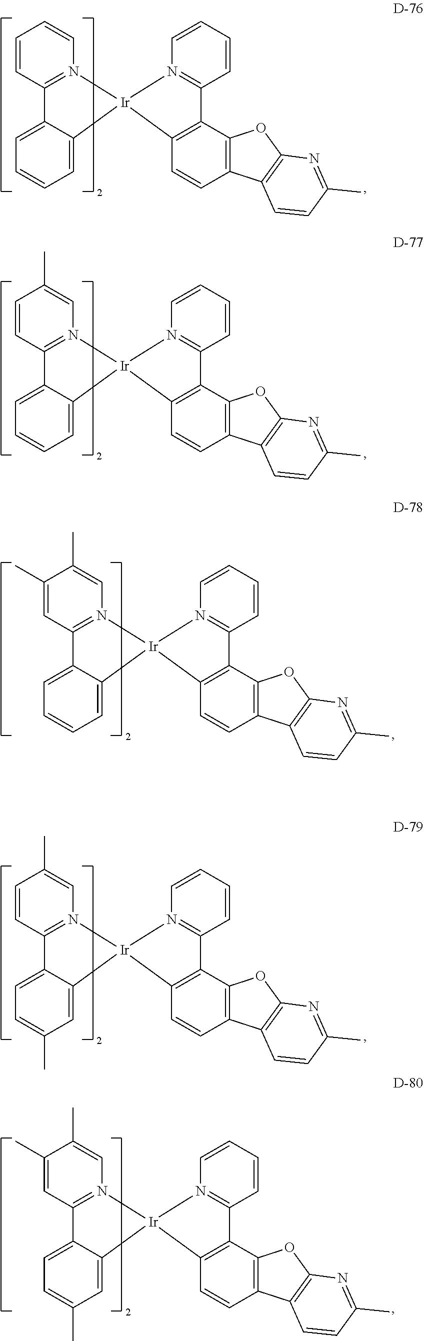

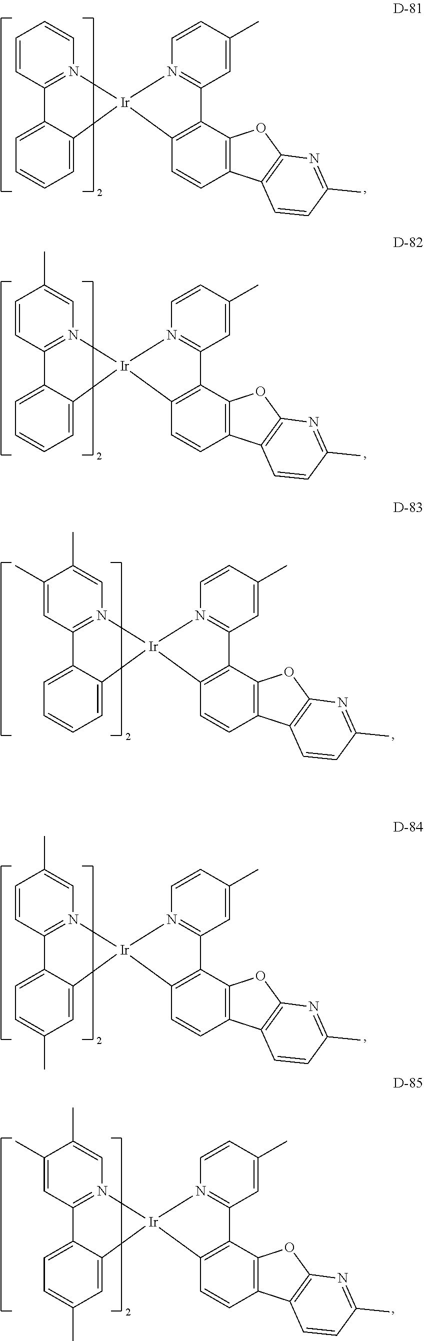

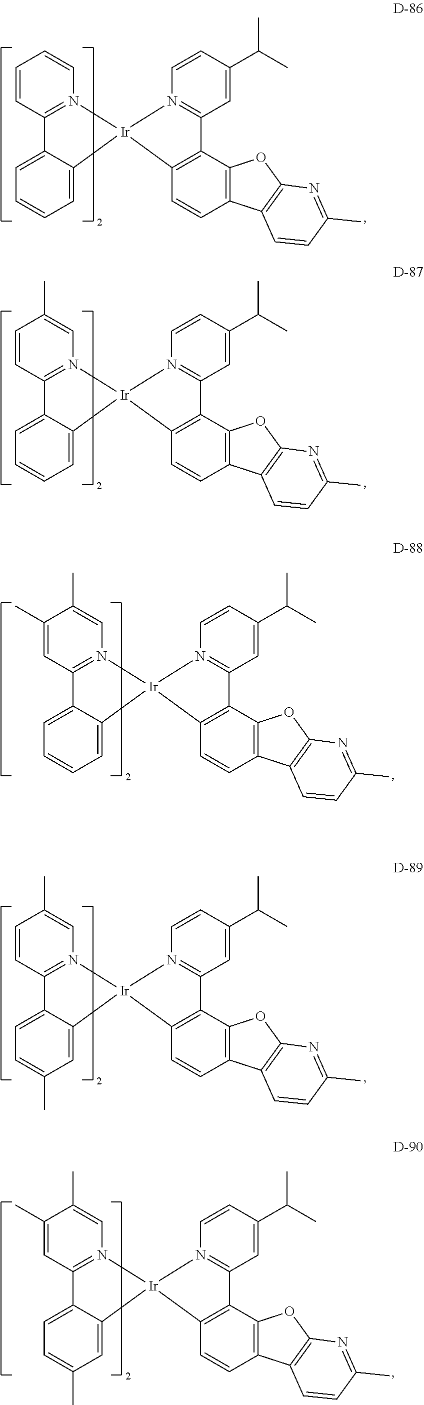

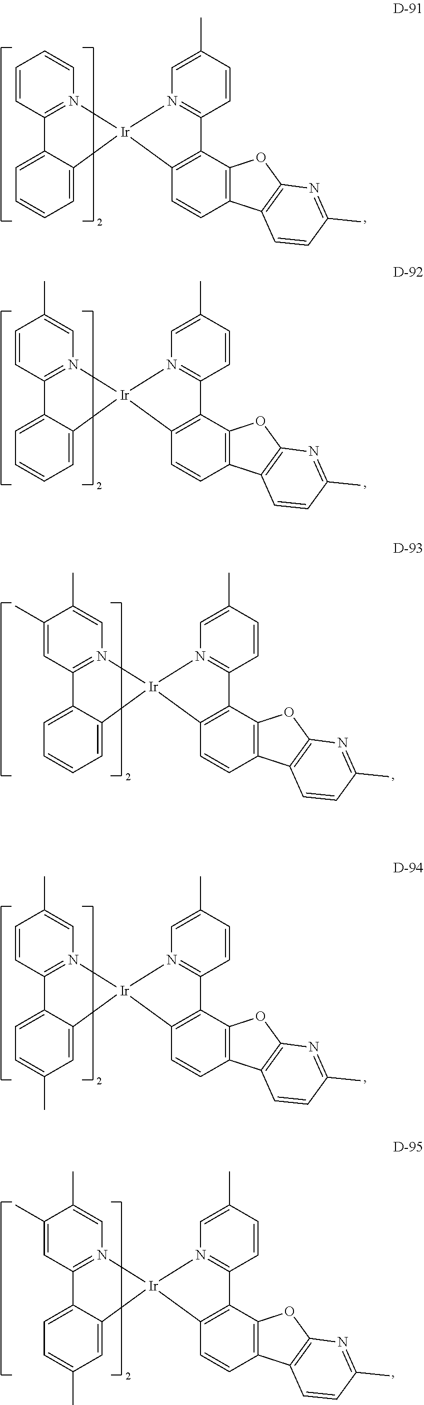

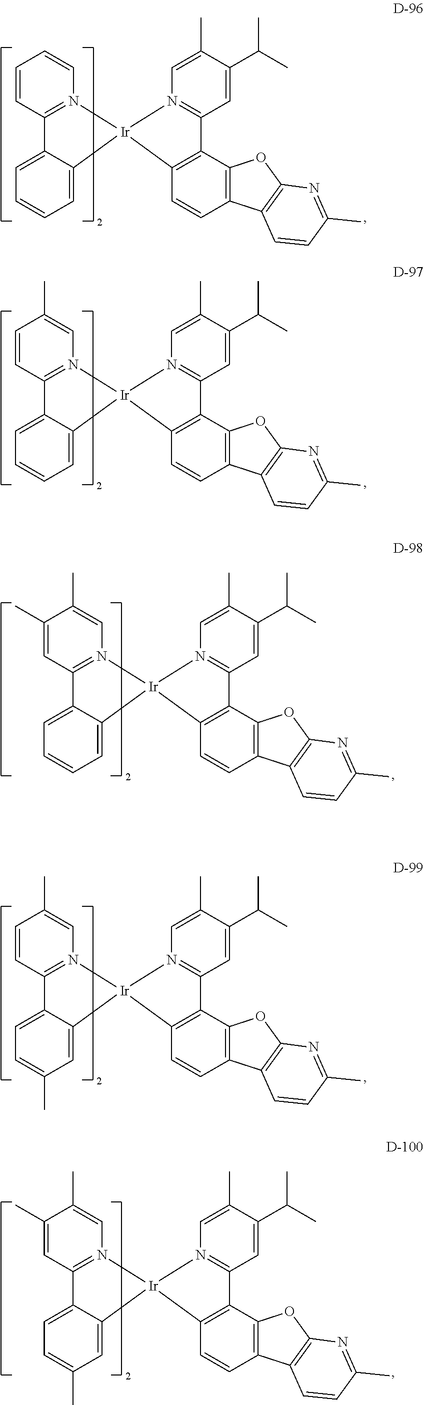

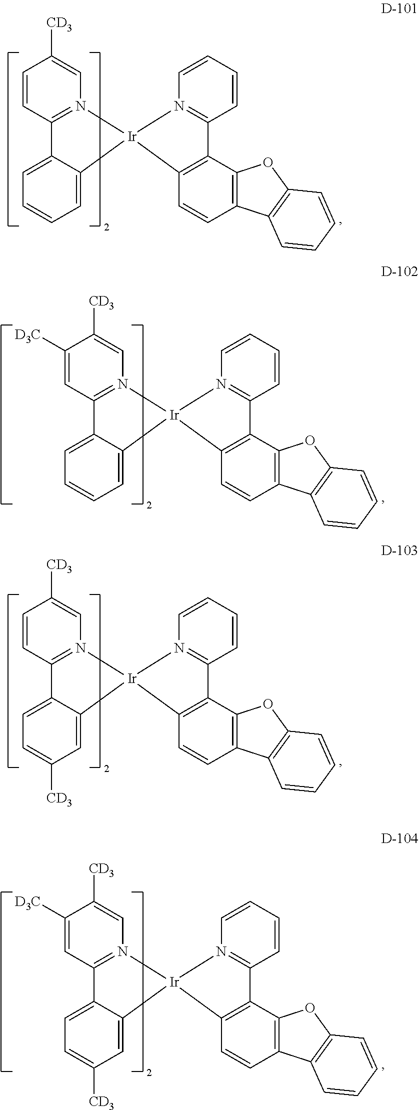

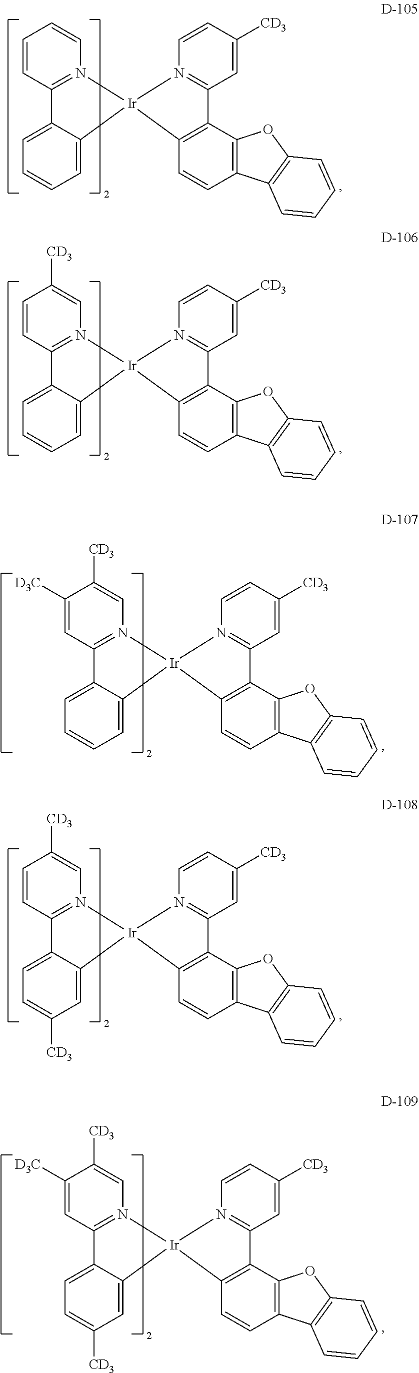

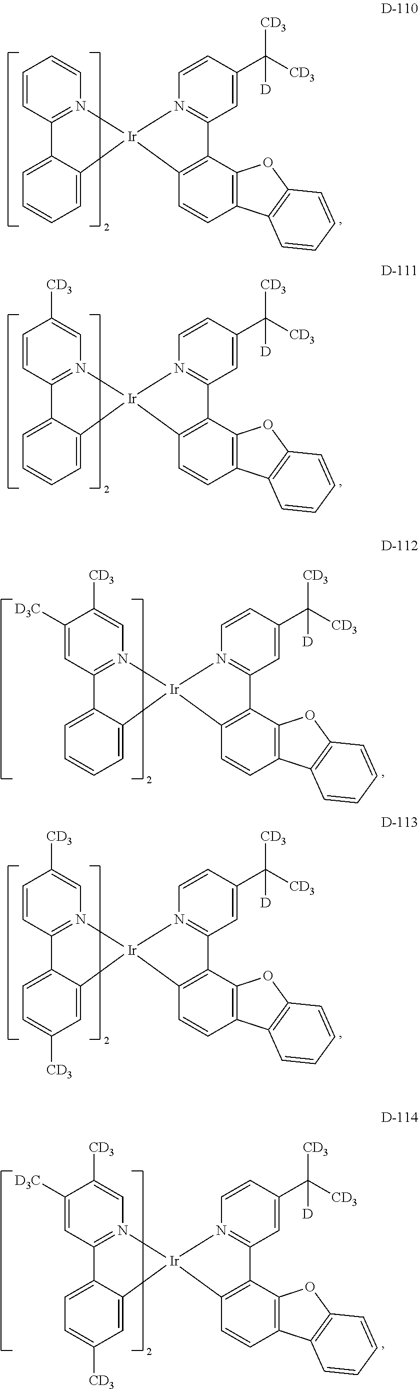

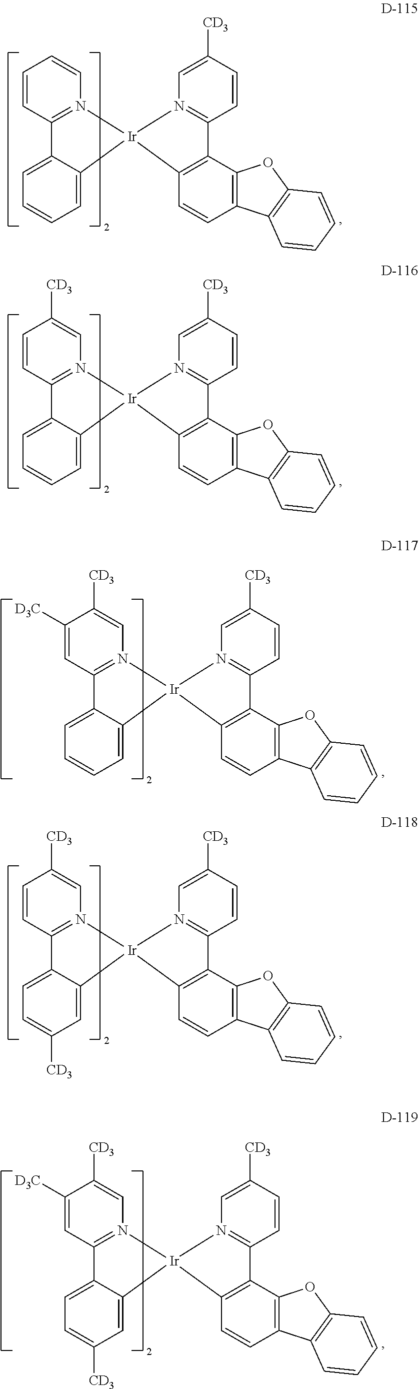

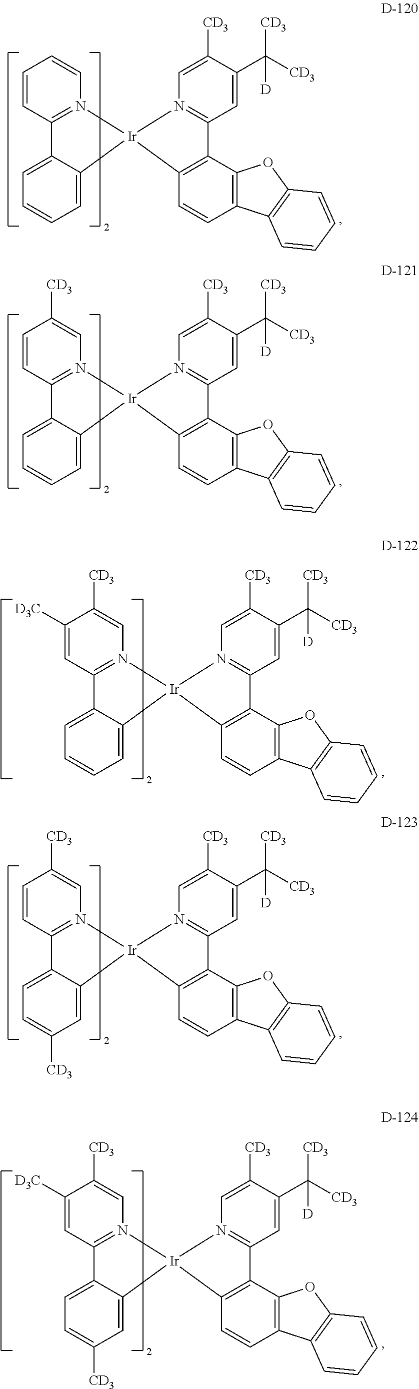

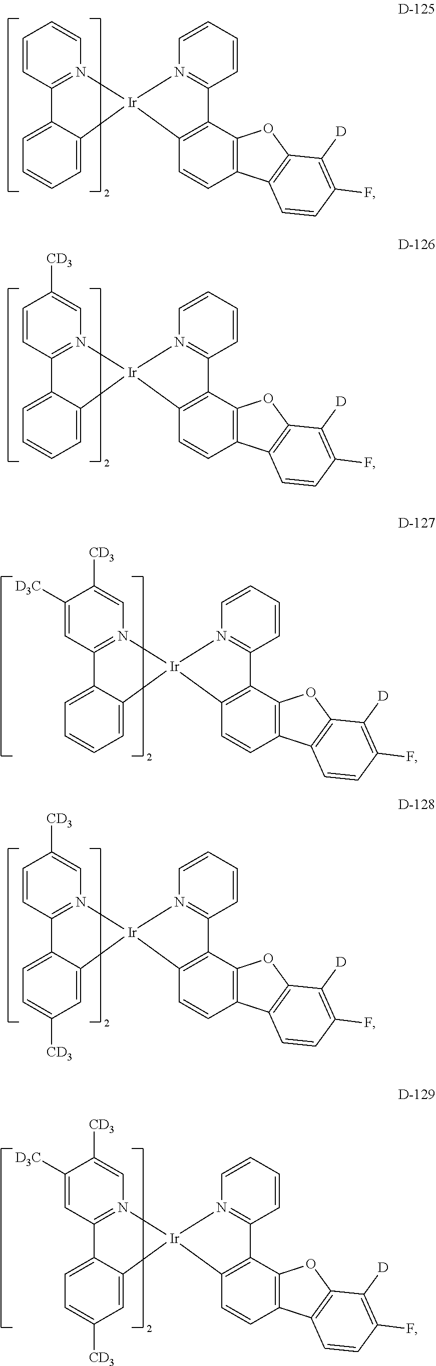

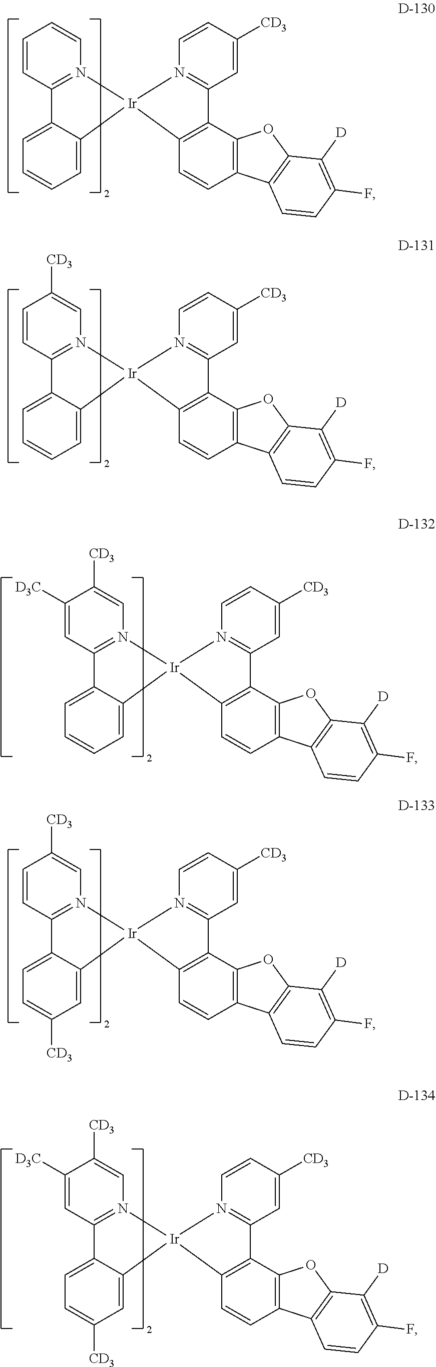

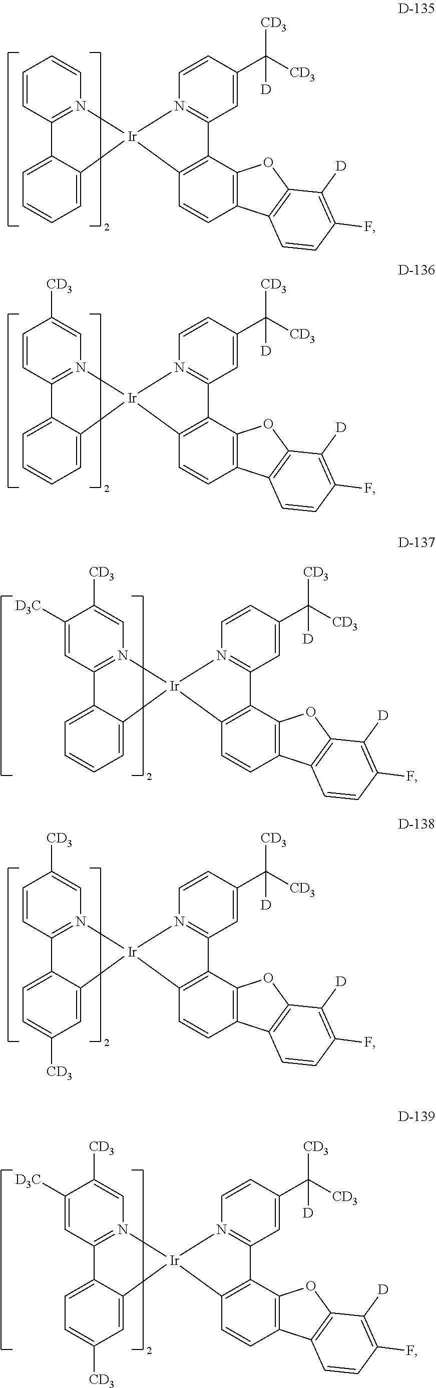

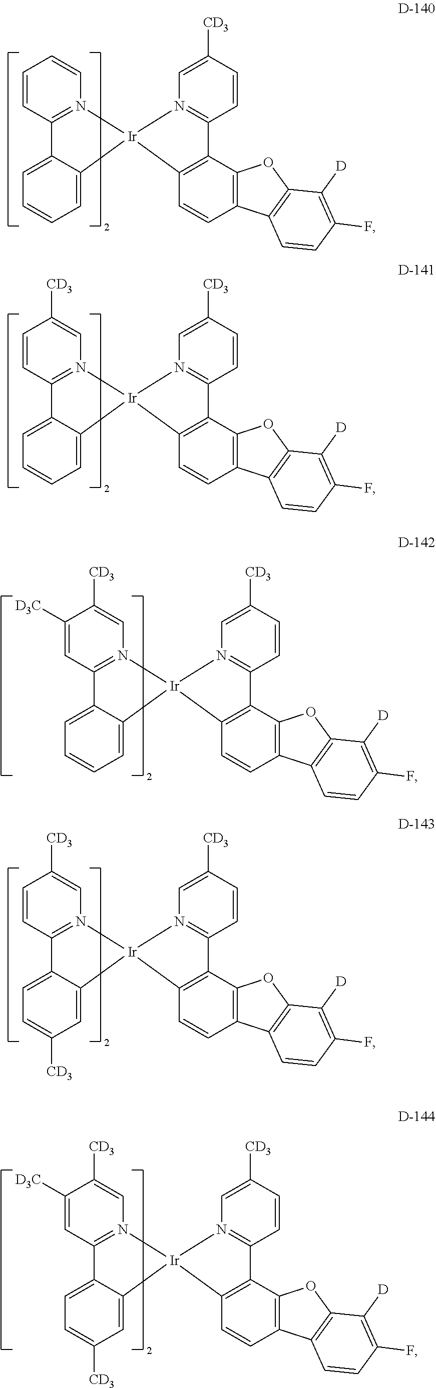

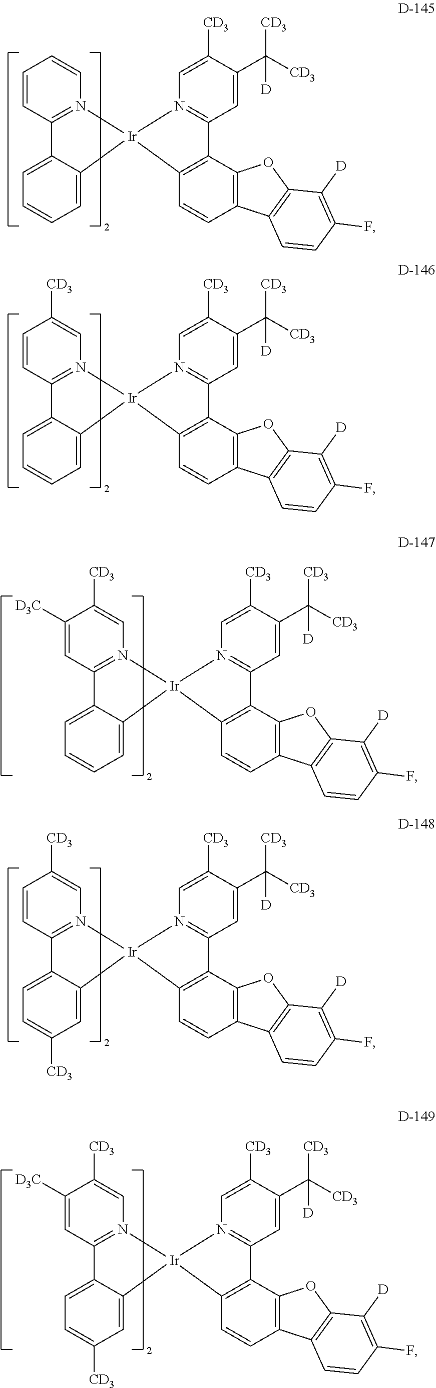

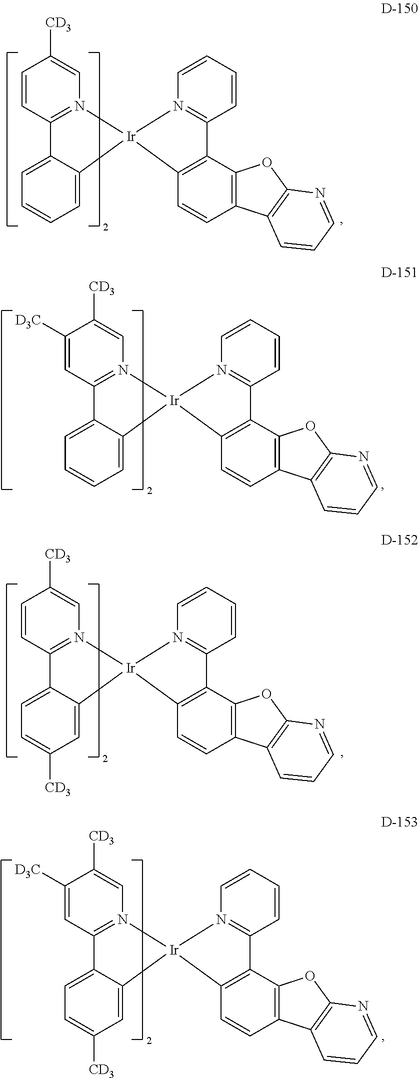

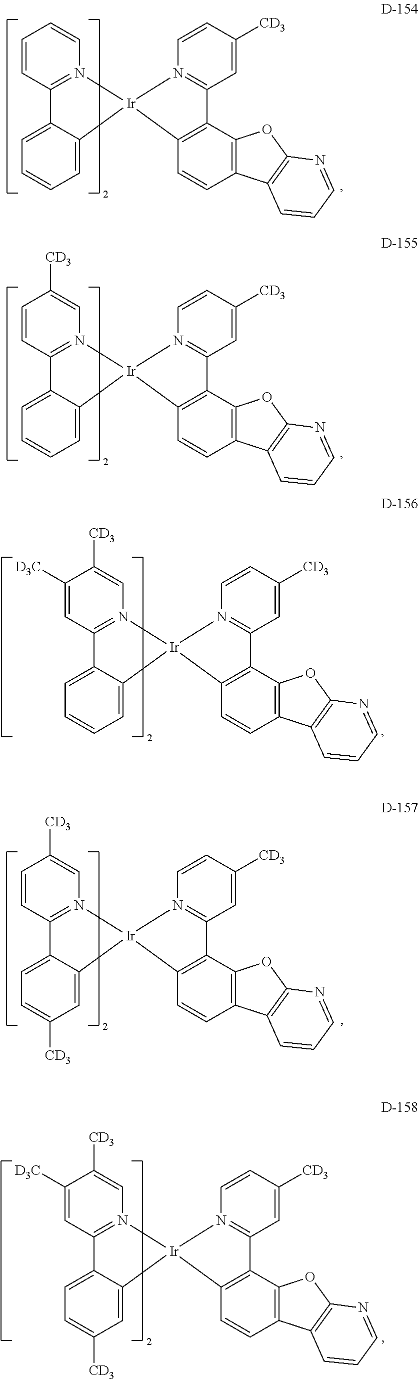

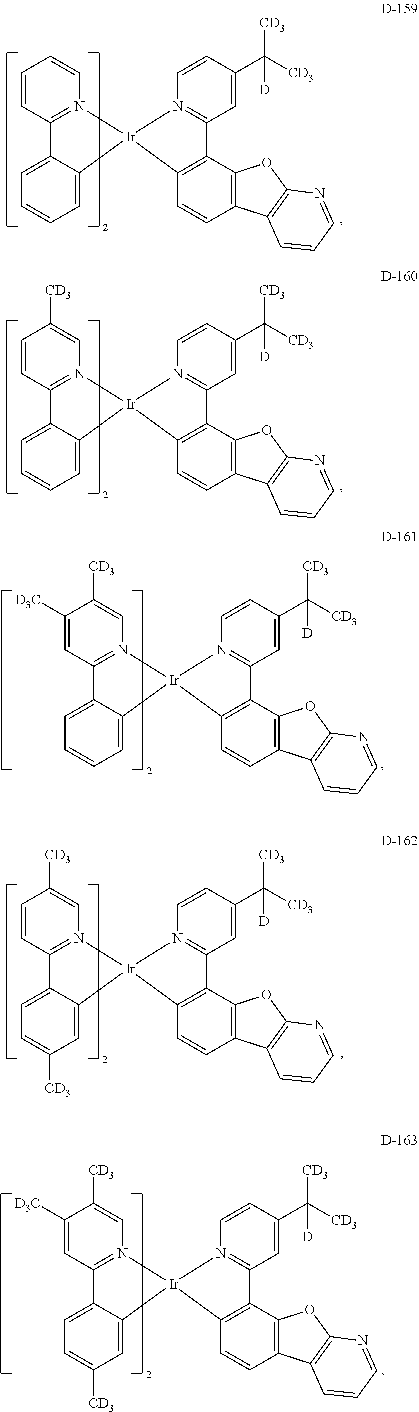

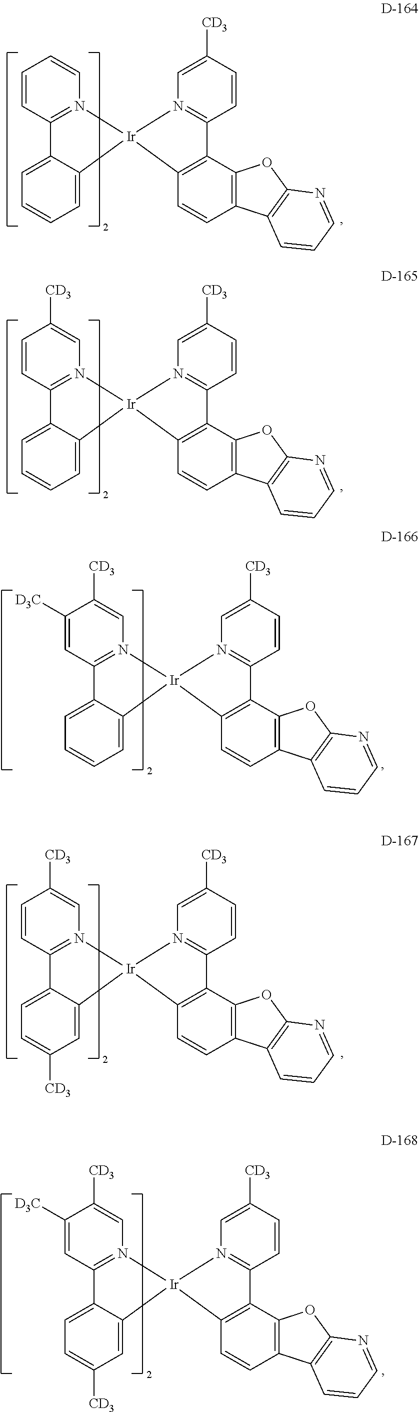

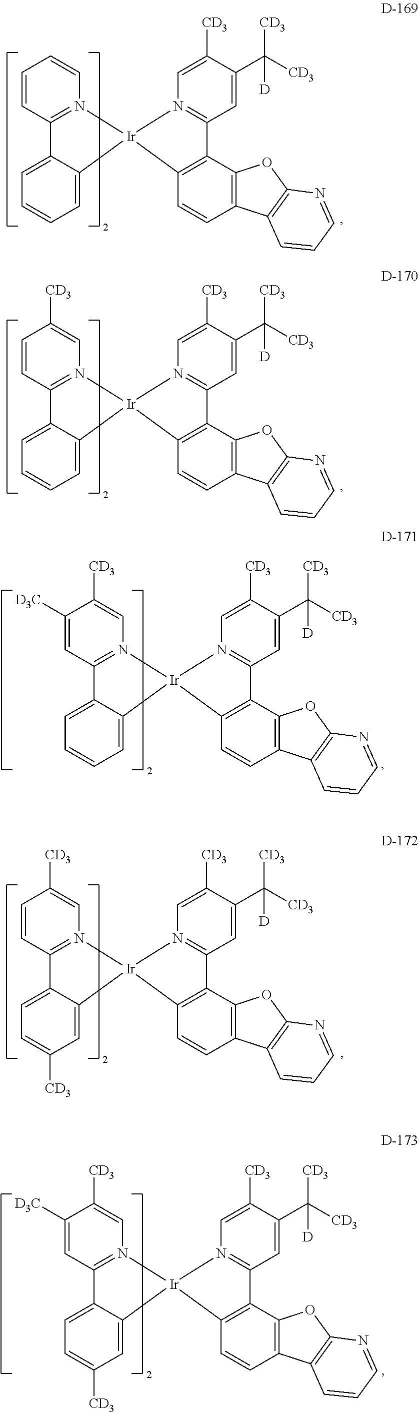

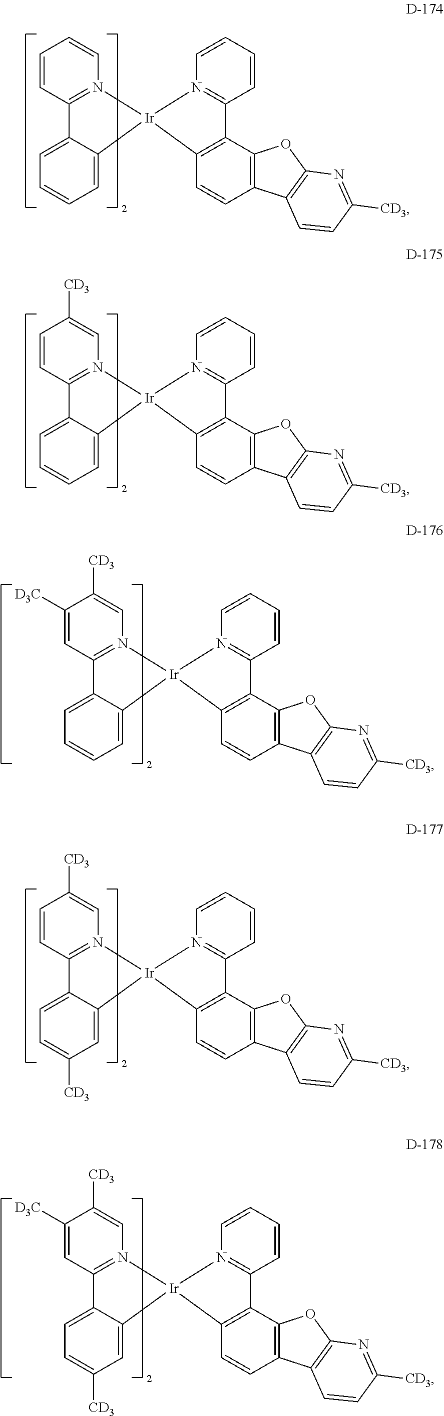

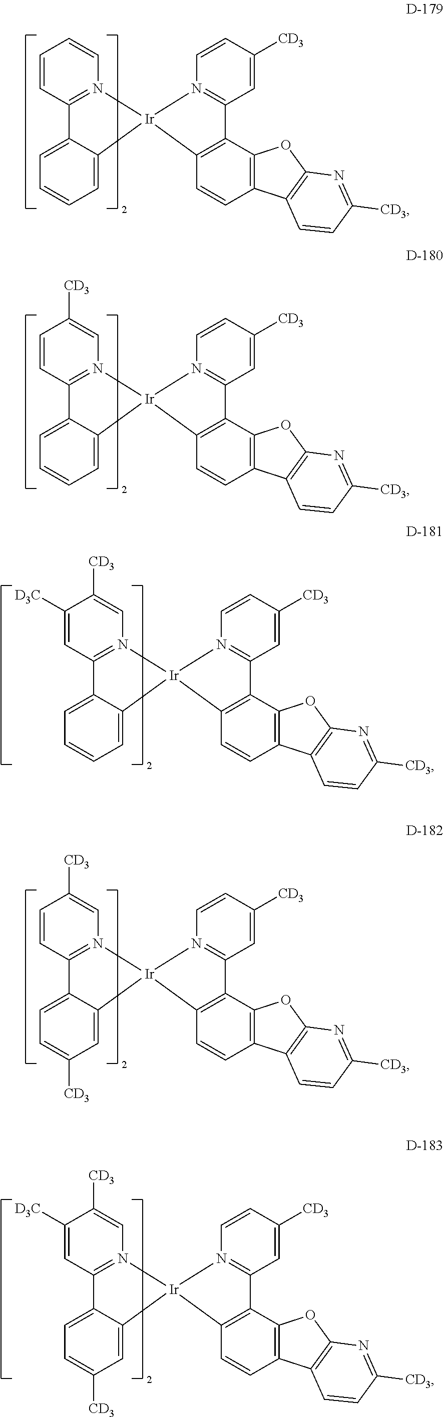

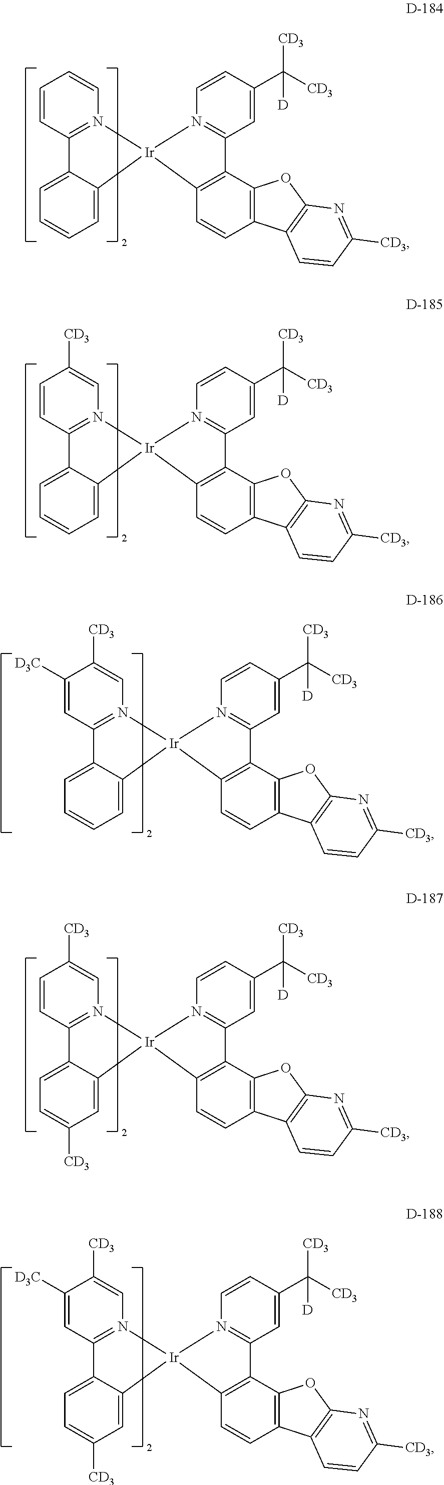

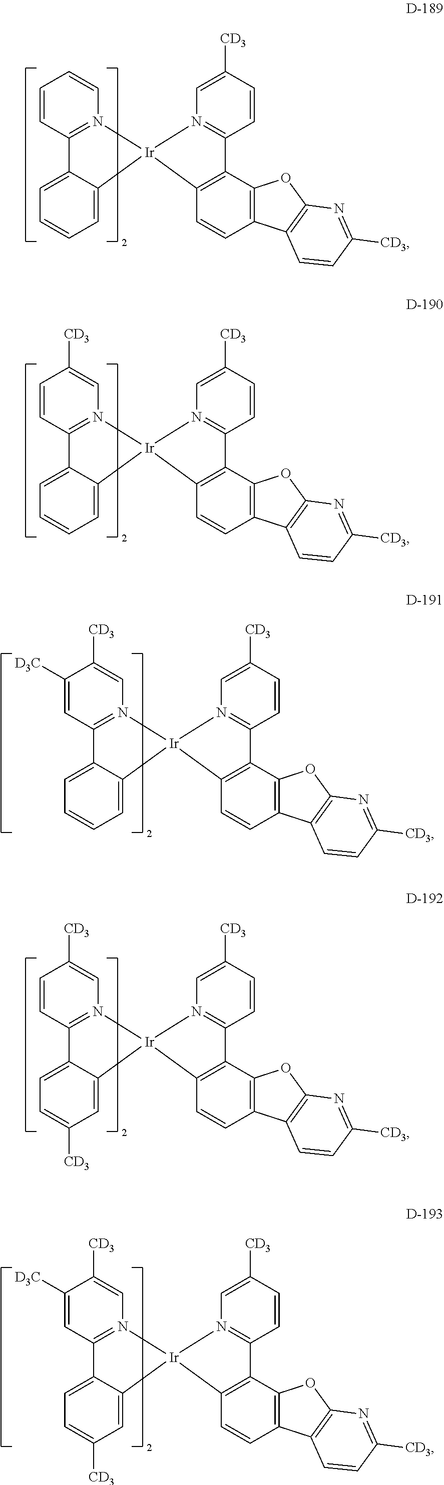

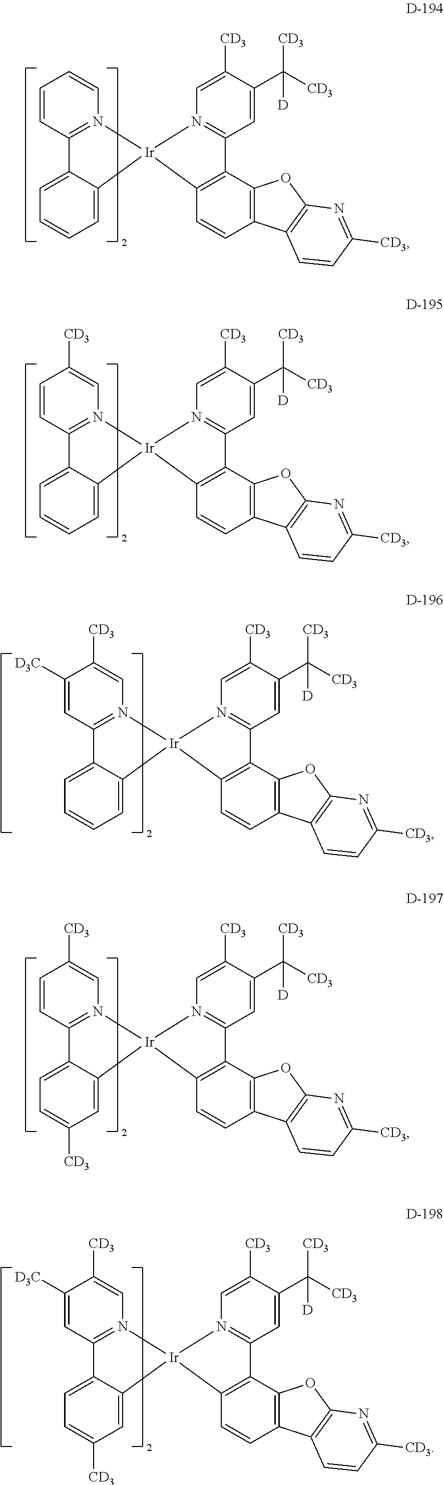

According to another embodiment of the present disclosure, a display module is also disclosed, which comprises an organic electroluminescent device. Wherein the specific structure of the organic electroluminescent device, refer to any of the above embodiments.

According to another embodiment of the present disclosure, a formulation is also disclosed, which comprising host materials and a dopant material, wherein the host materials comprises a first host compound represented by formula 1 and a second host compound represented by formula 2, wherein the dopant material comprises a dopant compound represented by formula 3:

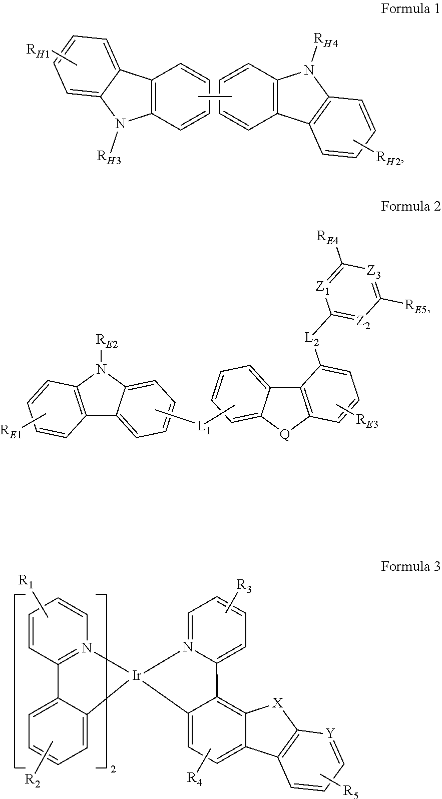

wherein,

R_{H1}, R_{H2}, R_{E1}, R_{E3}, R₁ to R₅ may represent mono substitution, multiple substitutions or no substitution; when R_{H1}, R_{H2}, R_{E1}, R_{E3}, R₁ to R₅ represent multiple substitution, each of R_{H1}, R_{H2}, R_{E1}, R_{E3}, R₁ to R₅ may be the same or different;

X and Q are each independently selected from O or S;

Z₁, Z₂, Z₃ and Y are each independently selected from CR or N;

L₁ and L₂ are each independently selected from the group consisting of a single bond, a substituted or unsubstituted arylene having 6 to 30 carbon atoms, a substituted or unsubstituted heteroarylene having 3 to 30 carbon atoms, and combinations thereof;

R₁ to R₅, R_{H1} to R_{H4}, R_{E1} to R_{E5} and R are each independently selected from the group consisting of hydrogen, deuterium, halogen, a substituted or unsubstituted alkyl group having 1 to 20 carbon atoms, a substituted or unsubstituted cycloalkyl group having 3 to 20 ring carbon atoms, a substituted or unsubstituted heteroalkyl group having 1 to 20 carbon atoms, a substituted or unsubstituted arylalkyl group having 7 to 30 carbon atoms, a substituted or unsubstituted alkoxy group having 1 to 20 carbon atoms, a substituted or unsubstituted aryloxy group having 6 to 30 carbon atoms, a substituted or unsubstituted alkenyl group having 2 to 20 carbon atoms, a substituted or unsubstituted aryl group having 6 to 30 carbon atoms, a substituted or unsubstituted heteroaryl group having 3 to 30 carbon atoms, a substituted or unsubstituted alkylsilyl group having 3 to 20 carbon atoms, a substituted or unsubstituted arylsilyl group having 6 to 20 carbon atoms, a substituted or unsubstituted amino group having 0 to 20 carbon atoms, an acyl group, a carbonyl group, a carboxylic acid group, an ester group, a nitrile group, an isonitrile group, a thiolalkyl group, a sulfinyl group, a sulfonyl group, a phosphino group, and combinations thereof;

any adjacent substitution may be optionally joined to form a ring.

Combination with Other Materials

The materials described in the present disclosure for a particular layer in an organic light emitting device may be used in combination with various other materials present in the device. The combinations of these materials are described in more detail in U.S. Pat. App. No. 20160359122A1 at paragraphs 0132-0161, which is incorporated by reference herein in its entirety. The materials described or referred to the disclosure are non-limiting examples of materials that may be useful in combination with the materials disclosed herein, and one of skill in the art may readily consult the literature to identify other materials that may be useful in combination.

The materials described herein as useful for a particular layer in an organic light emitting device may be used in combination with a variety of other materials present in the device. For example, the emitting layer materials disclosed herein may be used in combination with a wide variety of transporting layers, blocking layers, injection layers, electrodes and other layers that may be present. The combination of these materials is described in detail in paragraphs 0080-0101 of U.S. Pat. App. No. 20150349273, which is incorporated by reference herein in its entirety. The materials described or referred to the disclosure are non-limiting examples of materials that may be useful in combination with the materials disclosed herein, and one of skill in the art may readily consult the literature to identify other materials that may be useful in combination.

The manufacturing method of the organic electroluminescent device is not limited, and the manufacturing methods of the following examples are only examples, and should not be construed as limiting. Those skilled in the art will be able to rationally improve the preparation methods of the following examples in accordance with the prior art. Illustratively, the ratio of the various materials in the emitting layer is not particularly limited, and those skilled in the art may reasonably select within a certain range according to the prior art, for example, based on the total weight of the emitting layer material, the first host compound accounts for 10% to 90%, the second host compound accounts for 10% to 90%, the dopant compound accounts for 1% to 60% or the preferred dopant compound accounts for 3% to 30%. More preferably, the first host compound comprises from 20% to 60%, the second host compound comprises from 20% to 60%, and the dopant compound comprises from 3% to 30%. The characteristics of the light-emitting device prepared in the examples were tested using devices conventional in the art, using methods well known to those skilled in the art. Since those skilled in the art are aware of the above-mentioned device usage, test methods, and the like, the inherent data of the sample may be obtained with certainty and unaffectedness, and thus the above related content will not be further described in this patent.

### Example 1-1: Preparation of an Organic Electroluminescent Device Comprising a Material Combination of the Disclosure

A glass substrate with 80 nm thick of ITO electrode was cleaned and subjected to UV ozone and oxygen plasma treatment. After the treatments, the substrate was dried in a nitrogen-filled glovebox to remove moisture, the substrate was then mounted on a substrate holder and loaded into a vacuum chamber. The organic layers specified below were deposited in sequence by thermal vacuum deposition on the ITO anode at a rate of 0.2-2 Å/s under a vacuum degree of around 10⁻⁸ torr. Compound HI was used as the hole injection layer (HIL), the thickness is 100 Å. Compound HT was used as the hole transporting layer (HTL), the thickness is 350 Å. Compound H-25 was used as the electron blocking layer (EBL), the thickness is 50 Å. Then the Compound D-174 was then doped in the first host Compound H-25 and the second host compound E-1 to co-deposited as the emitting layer (EML), the total thickness is 400 Å, the weight ratio of compound H-25 to compound E-1 is 1:1, the dopant compound D-174 accounts for 10% of the total weight of the emitting layer. Compound Host 1 was used as hole blocking layer (HBL), the thickness is 100 Å. On HBL, Compound ET and 8-hydroxyquinoline-lithium (Liq) were co-deposition as an electron transporting layer (ETL), wherein Liq accounts for 60% of the total weight of the ETL layer, the total thickness of ETL is 350 Å. Finally, 10 Å-thick 8-hydroxyquinoline-lithium (Liq) was deposited as the electron injection layer (EIL) and 1200 Å of Al was deposited as the cathode. Then, the device was transferred back to the glovebox and encapsulated with a glass lid and a moisture getter to complete the device.

Comparative Example 1-1 to 1-3: fabricated in the same manner as in Example 1-1, except that the host materials in light-emitting layer of Comparative Examples 1-1 to 1-3 in Table 1 were used, and the weight ratio of the host material to the dopant was adjusted to 80:20, which is the optimal weight ratio of the device using one-component host and dopant that may obtain the best performance of the device.

Comparative Example 1-4 to 1-6: fabricated in the same manner as in Example 1-1, except that the host materials in light-emitting layer of Comparative Examples 1-4 to 1-6 in Table 1 were used.

The detailed partial structures of devices and thicknesses are shown in table 1. In the layers in which more than one material was used, they were obtained by doping different compounds in the weight ratios described therein.

TABLE 1

| Device ID | HIL | HTL | EBL | EML | HBL | ETL |
|---|---|---|---|---|---|---|
| Example 1-1 | Compound | Compound | Compound | Compound H-25: | Compound | Compound |
| | HI | HT | H-25 | Compound E-1: | Host 1 | ET:Liq (40:60) |
| | (100 Å) | (350 Å) | (50 Å) | Compound D-174 | (100 Å) | (350 Å) |
| | | | | (45:45:10)(400 Å) | | |
| Comparative | Compound | Compound | Compound | Compound H-25: | Compound | Compound |
| Example 1-1 | HI | HT | H-25 | Compound D-174 | Host 1 | ET:Liq (40:60) |
| | (100 Å) | (350 Å) | (50 Å) | (80:20)(400 Å) | (100 Å) | (350 Å) |
| Comparative | Compound | Compound | Compound | Compound Host 1: | Compound | Compound |
| Example 1-2 | HI | HT | H-25 | Compound D-174 | Host 1 | ET:Liq (40:60) |
| | (100 Å) | (350 Å) | (50 Å) | (80:20)(400 Å) | (100 Å) | (350 Å) |
| Comparative | Compound | Compound | Compound | Compound E-1: | Compound | Compound |
| Example 1-3 | HI | HT | H-25 | Compound D-174 | Host 1 | ET:Liq (40:60) |
| | (100 Å) | (350 Å) | (50 Å) | (80:20)(400 Å) | (100 Å) | (350 Å) |
| Comparative | Compound | Compound | Compound | Compound H-25: | Compound | Compound |
| Example 1-4 | HI | HT | H-25 | Compound Host 2: | Host 1 | ET:Liq (40:60) |
| | (100 Å) | (350 Å) | (50 Å) | Compound D-174 | (100 Å) | (350 Å) |
| | | | | (45:45:10)(400 Å) | | |
| Comparative | Compound | Compound | Compound | Compound H-25: | Compound | Compound |
| Example 1-5 | HI | HT | H-25 | Compound Host 1: | Host 1 | ET:Liq (40:60) |
| | (100 Å) | (350 Å) | (50 Å) | Compound D-174 | (100 Å) | (350 Å) |
| | | | | (45:45:10)(400 Å) | | |
| Comparative | Compound | Compound | Compound | Compound Host 3: | Compound | Compound |
| Example 1-6 | HI | HT | H-25 | Compound Host 1: | Host 1 | ET:Liq (40:60) |
| | (100 Å) | (350 Å) | (50 Å) | Compound D-174 | (100 Å) | (350 Å) |
| | | | | (45:45:10)(400 Å) | | |

Structure of the partial materials used in the devices are shown as below:

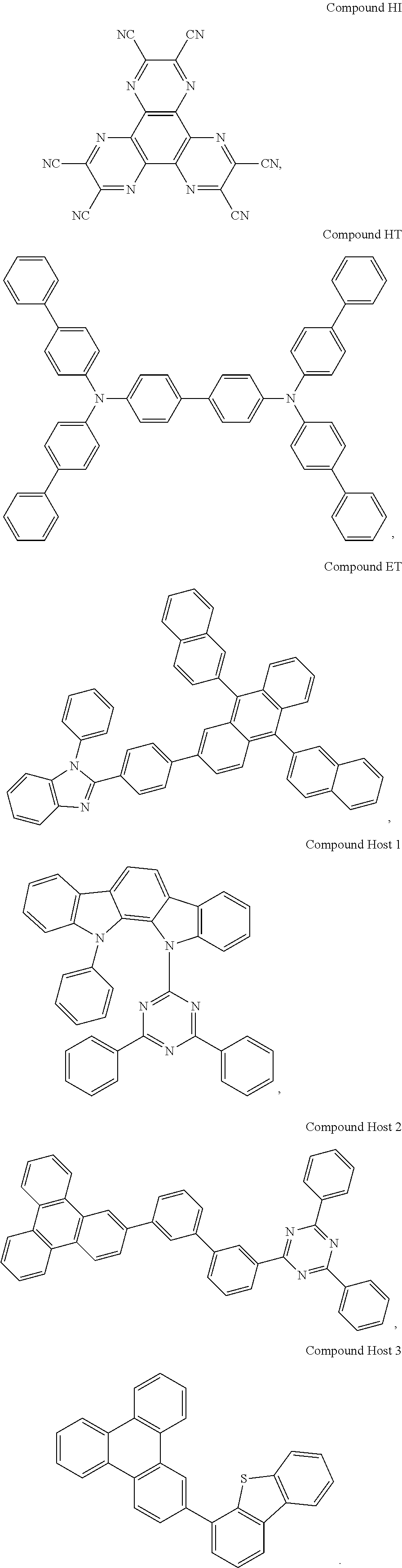 [SMILES: N#Cc1nc2c3nc(C#N)c(C#N)nc3c3nc(C#N)c(C#N)nc3c2nc1C#N ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2nc(-c3ccccc3)nc(-n3c4ccccc4c4ccc5c6ccccc6n(-c6ccccc6)c5c43)n2)cc1 ; c1ccc(-c2nc(-c3ccccc3)nc(-c3cccc(-c4cccc(-c5ccc6c7ccccc7c7ccccc7c6c5)c4)c3)n2)cc1 ; c1ccc2c(c1)sc1c(-c3ccc4c5ccccc5c5ccccc5c4c3)cccc12]

The test results of Example 1-1 and Comparative Example 1-1 to Comparative Example 1-6 are listed in table 2. The chromaticity coordinate (CIE), λₘₐₓ and half-width (FWHM) in table 2 were tested at 1000 nits, voltage, external quantum efficiency (EQE) and current efficiency (CE) were tested under current density of 15 mA/cm², the lifetime is the time required for the initial 10000 nits luminance to decay to 95% of the initial luminance.

TABLE 2

| Device | CIE | | λₘₐₓ | FWHM | Voltage | EQE | CE | LT95 |
|---|---|---|---|---|---|---|---|---|
| ID | (x y) | | (nm) | (nm) | (v) | (%) | (cd/A) | (h) |
| Example | 0.338 | 0.632 | 527 | 58.3 | 4.19 | 21.87 | 84 | 1260 |
| 1-1 | | | | | | | | |
| Com- | 0.343 | 0.629 | 529 | 59.8 | 5.35 | 16.98 | 66 | 21 |
| parative | | | | | | | | |
| Example | | | | | | | | |
| 1-1 | | | | | | | | |
| Com- | 0.341 | 0.631 | 529 | 56.4 | 4.19 | 19.42 | 75 | 334 |
| parative | | | | | | | | |
| Example | | | | | | | | |
| 1-2 | | | | | | | | |
| Com- | 0.351 | 0.623 | 530 | 60.3 | 4.03 | 18.52 | 71 | 251 |
| parative | | | | | | | | |
| Example | | | | | | | | |
| 1-3 | | | | | | | | |
| Com- | 0.333 | 0.635 | 527 | 58.1 | 5.24 | 21.03 | 81 | 854 |
| parative | | | | | | | | |
| Example | | | | | | | | |
| 1-4 | | | | | | | | |
| Com- | 0.332 | 0.637 | 527 | 56.4 | 4.21 | 21.46 | 83 | 961 |
| parative | | | | | | | | |
| Example | | | | | | | | |
| 1-5 | | | | | | | | |
| Com- | 0.339 | 0.632 | 529 | 57.7 | 4.63 | 22.20 | 85 | 182 |
| parative | | | | | | | | |
| Example | | | | | | | | |
| 1-6 | | | | | | | | |

Table 2 shows the test results of electroluminescent devices with different host materials and dopant D-174. As shown in the table, the color coordinate of Example 1-1 is (0.338, 0.632), the λₘₐₓ is 527 nm, the FWHM is 58.3 nm, the voltage under 15 mA/cm² is 4.19 V, the EQE is 21.87%, and the CE is 84 cd/A, lifetime of LT95 under 10000 nits is 1260 hours. The performance parameters of Comparative Example 1-1 are all inferior than Example 1-1, the spectrum red-shift, especially the lifetime of LT95 is only 21 hours which is far less than Example 1-1. Although the FWHM of Comparative Example 1-2 is only 56.4 nm, which is slightly narrower than that of Example 1-1, and the voltage is the same as that of Example 1-1, the EQE, CE, and the lifetime of LT95 are all inferior than Example 1-1. Although the voltage of Comparative Example 1-3 is only 4.03 V, the spectrum compared with Example 1-1 is red-shift, and EQE, CE, and the lifetime of LT95 are all inferior than Example 1-1. The λₘₐₓ and the FWHM of Comparative Example 1-4 are both close to Example 1-1, but the voltage is as high as 5.24 V, which is larger than Example 1-1 by more than 1 V, and the EQE, CE, and the lifetime of LT95 are still lower than Example 1-1. The spectra of Comparative Example 1-5 is slightly blue-shift than Example 1-1, and the FWHM is 56.4 nm which is slightly narrower than Example 1-1. The three parameters of voltage, EQE, and CE are close to Example 1-1, but the lifetime is significantly lower than Example 1-1, differed by 299 hours. The spectra of Comparative Example 1-6 is red-shifted than Example 1-1, although the EQE and CE are slightly higher than Example 1-1, but the voltage is as high as 4.63 V, exceeding the voltage of Example 1-1 by 0.44 V, and the lifetime of LT95 is only 182 hours which is much smaller than the life of Example 1-1. These results indicate that the use of a combination of emitting layer materials comprising the present disclosure may improve the spectrum and significantly improve the overall performance of the device, such as lowering the voltage, improving efficiency and lifetime.

Example 2-1: fabricated in the same manner as in Example 1-1, except that the dopant compound used in the light-emitting layer is dopant compound D-125, which accounts for 8% of the total weight of the light-emitting layer.

Comparative Example 2-1 to 2-3: fabricated in the same manner as in Example 2-1, except that the host materials in light-emitting layer of Comparative Examples 2-1 to 2-3 in Table 3 were used, and the weight ratio of the host material to the dopant was adjusted to 80:20, which is the optimal weight ratio of the device using one-component host and dopant that may obtain the best performance of the device.

Comparative Example 2-4 to 2-6: fabricated in the same manner as in Example 2-1, except that the host materials in light-emitting layer of Comparative Examples 2-4 to 2-6 in Table 3 were used.

The detailed partial structures of devices and thicknesses are shown in the table. In the layers in which more than one material was used, they were obtained by doping different compounds in the weight ratios described therein.

TABLE 3

| Device ID | HIL | HTL | EBL | EML | HBL | ETL |
|---|---|---|---|---|---|---|
| Example 2-1 | Compound | Compound | Compound | Compound H-25: | Compound | Compound |
| | HI | HT | H-25 | Compound E-1: | Host 1 | ET:Liq (40:60) |
| | (100 Å) | (350 Å) | (50 Å) | Compound D-125 | (100 Å) | (350 Å) |
| | | | | (46:46:8)(400 Å) | | |
| Comparative | Compound | Compound | Compound | Compound H-25: | Compound | Compound |
| Example 2-1 | HI | HT | H-25 | Compound D-125 | Host 1 | ET:Liq (40:60) |
| | (100 Å) | (350 Å) | (50 Å) | (80:20)(400 Å) | (100 Å) | (350 Å) |
| Comparative | Compound | Compound | Compound | Compound Host 1: | Compound | Compound |
| Example 2-2 | HI | HT | H-25 | Compound D-125 | Host 1 | ET:Liq (40:60) |
| | (100 Å) | (350 Å) | (50 Å) | (80:20)(400 Å) | (100 Å) | (350 Å) |
| Comparative | Compound | Compound | Compound | Compound E-1: | Compound | Compound |
| Example 2-3 | HI | HT | H-25 | Compound D-125 | Host 1 | ET:Liq (40:60) |
| | (100 Å) | (350 Å) | (50 Å) | (80:20)(400 Å) | (100 Å) | (350 Å) |
| Comparative | Compound | Compound | Compound | Compound H-25: | Compound | Compound |
| Example 2-4 | HI | HT | H-25 | Compound Host 2: | Host 1 | ET:Liq (40:60) |
| | (100 Å) | (350 Å) | (50 Å) | Compound D-125 | (100 Å) | (350 Å) |
| | | | | (46:46:8)(400 Å) | | |
| Comparative | Compound | Compound | Compound | Compound H-25: | Compound | Compound |
| Example 2-5 | HI | HT | H-25 | Compound Host 1: | Host 1 | ET:Liq (40:60) |
| | (100 Å) | (350 Å) | (50 Å) | Compound D-125 | (100 Å) | (350 Å) |
| | | | | (46:46:8)(400 Å) | | |
| Comparative | Compound | Compound | Compound | Compound Host 3: | Compound | Compound |
| Example 2-6 | HI | HT | H-25 | Compound Host 1: | Host 1 | ET:Liq (40:60) |
| | (100 Å) | (350 Å) | (50 Å) | Compound D-125 | (100 Å) | (350 Å) |
| | | | | (46:46:8)(400 Å) | | |

The compounds HI, HT, ET, Host 1, Host 2 and Host 3 used in the devices of Table 3 have the same structures as in Table 1.

The test results of Example 2-1 and Comparative Example 2-1 to Comparative Example 2-6 are listed in table 4. the chromaticity coordinate (CIE), λₘₐₓ and half-width (FWHM) in table 4 were tested at 1000 nits, voltage, external quantum efficiency (EQE) and current efficiency (CE) were tested under current density of 15 mA/cm², the lifetime is the time required for the initial 10000 nits luminance to decay to 95% of the initial luminance.

TABLE 4

| Device | CIE | | λₘₐₓ | FWHM | Voltage | EQE | CE | LT95 |
|---|---|---|---|---|---|---|---|---|
| ID | (x y) | | (nm) | (nm) | (v) | (%) | (cd/A) | (h) |
| Example | 0.345 | 0.627 | 529 | 60.7 | 4.03 | 21.41 | 82 | 873 |
| 2-1 | | | | | | | | |
| Com- | 0.348 | 0.626 | 530 | 60.2 | 5.10 | 16.70 | 64 | 76 |
| parative | | | | | | | | |
| Example | | | | | | | | |
| 2-1 | | | | | | | | |
| Com- | 0.349 | 0.625 | 530 | 59.2 | 4.31 | 20.11 | 77 | 309 |
| parative | | | | | | | | |
| Example | | | | | | | | |
| 2-2 | | | | | | | | |
| Com- | 0.361 | 0.615 | 532 | 63.8 | 4.15 | 19.02 | 72 | 222 |
| parative | | | | | | | | |
| Example | | | | | | | | |
| 2-3 | | | | | | | | |
| Com- | 0.337 | 0.632 | 527 | 59.9 | 5.16 | 20.83 | 80 | 624 |
| parative | | | | | | | | |
| Example | | | | | | | | |
| 2-4 | | | | | | | | |
| Com- | 0.340 | 0.630 | 528 | 59.2 | 4.11 | 21.49 | 82 | 691 |
| parative | | | | | | | | |
| Example | | | | | | | | |
| 2-5 | | | | | | | | |
| Com- | 0.345 | 0.627 | 529 | 60.5 | 4.60 | 21.30 | 81 | 117 |
| parative | | | | | | | | |
| Example | | | | | | | | |
| 2-6 | | | | | | | | |

Table 4 shows the test results of electroluminescent devices with different host materials and dopant D-125. The color coordinate of Example 2-1 is (0.345, 0.627), the λₘₐₓ is 529 nm, the FWHM is 60.7 nm, the voltage under 15 mA/cm² is 4.03 V, the EQE is 21.41%, and the CE is 82 cd/A, lifetime of LT95 under 10000 nits is 873 hours. The spectrum of Comparative Example 2-1 shows a slight red-shift, and the FWHM is close to that of Example 2-1, but the voltage is as high as 5.1 V which is higher than Example 2-1 by more than 1 V, and the EQE, CE, and lifetime of LT95 are far less than those in Example 2-1. The spectrum of Comparative Example 2-2 is also slightly red-shift, and the FWHM is slightly narrow, but the voltage is 4.31 V which is 0.28 V higher than Example 2-1, and the EQE, CE, and lifetime of LT95 are inferior to Example 2-1. The color coordinates of Comparative Example 2-3 are (0.361, 0.615), and the λₘₐₓ is 532 nm. The spectrum is significantly red-shifted as compared with Example 2-1, and although the voltage is close to Example 2-1, the EQE, CE, the lifetime of LT95 is far less than Example 2-1. The spectrum of Comparative Example 2-4 is blue-shifted, FWHM is slightly narrower, other parameters are less than Example 2-1, especially the voltage is as high as 5.16 V, which is 1.13 V higher than Example 2-1. In Comparative Example 2-5, the spectrum is slightly blue-shifted, the FWHM is slightly narrow, and the EQE is 21.49%, which is slightly higher than Example 2-1, and the CE is 82 cd/A which is the same as Example 2-1, but The lifetime of LT95 is only 691 hours, which is 182 hours less than Example 2-1. The spectrum, FWHM, EQE, and CE of Comparative Example 2-6 are all similar to Example 2-1, but the voltage of Comparative Example 2-5 is as high as 4.60 V which is 0.57 V higher than Example 2-1, and the lifetime of LT95 is only 117 hours which is 756 hours less than Example 2-1. These results indicate that the use of the combination of the emitting layer materials comprising the present disclosure may improve the voltage of the organic electroluminescent device and improve device efficiency and lifetime. Therefore, the electroluminescent device comprising the combination of the host and the dopant material disclosed in the present disclosure has more excellent overall performance.

In summary, the organic electroluminescent device disclosed in the present disclosure comprises at least one light emitting layer, wherein the light emitting layer comprising two host compounds and a phosphorescent dopant compound, wherein at least the first host compound comprises carbazole-carbazole structure, and the second host compound comprises a structure of formula 2, and in combination with a particular phosphorescent dopant compound. For a particular phosphorescent dopant included in a combination of materials, the combination of the particular materials described above may significantly improve the overall performance of the organic electroluminescent device, such as spectrum, voltage, luminous efficiency and lifetime, compared to the use of conventional host material combinations or single component host materials.

It is understood that the various embodiments described herein are by way of example only, and are not intended to limit the scope of the disclosure. The present disclosure as claimed may therefore include variations from the particular examples and preferred embodiments described herein, as will be apparent to one of skill in the art. Many of the materials and structures described herein may be substituted with other materials and structures without deviating from the spirit of the present disclosure. It is understood that various theories as to why the disclosure works are not intended to be limiting.

What is claimed is:

1. An organic electroluminescent device comprising:
an anode,
a cathode,
and at least one light emitting layer disposed between the anode and the cathode, the light emitting layer comprises host materials and a dopant material, wherein the host materials comprises a first host compound represented by Formula 1 and a second host compound represented by Formula 2, the dopant material comprises a dopant compound represented by Formula 3: 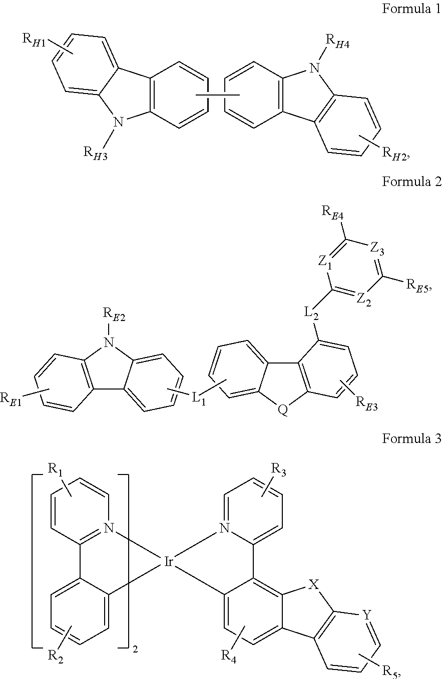
wherein
R_{H1}, R_{H2}, R_{E1}, R_{E3}, R₁ to R₅ may represent mono substitution, multiple substitutions or no substitution; when R_{H1}, R_{H2}, R_{E1}, R_{E3}, R₁ to R₅ represent multiple substitutions, each of R_{H1}, R_{H2}, R_{E1}, R_{E3}, R₁ to R₅ may be the same or different;
X and Q are each independently selected from O or S;
Z₁, Z₂, Z₃ and Y are each independently selected from CR or N;
L₁ and L₂ are each independently selected from the group consisting of a single bond, a substituted or unsubstituted arylene having 6 to 30 carbon atoms, a substituted or unsubstituted heteroarylene having 3 to 30 carbon atoms, and combinations thereof;
R₁ to R₅, R_{H1} to R_{H4}, R_{E1} to R_{E5} and R are each independently selected from the group consisting of hydrogen, deuterium, halogen, a substituted or unsubstituted alkyl group having 1 to 20 carbon atoms, a substituted or unsubstituted cycloalkyl group having 3 to 20 ring carbon atoms, a substituted or unsubstituted heteroalkyl group having 1 to 20 carbon atoms, a substituted or unsubstituted arylalkyl group having 7 to 30 carbon atoms, a substituted or unsubstituted alkoxy group having 1 to 20 carbon atoms, a substituted or unsubstituted aryloxy group having 6 to 30 carbon atoms, a substituted or unsubstituted alkenyl group having 2 to 20 carbon atoms, a substituted or unsubstituted aryl group having 6 to 30 carbon atoms, a substituted or unsubstituted heteroaryl group having 3 to 30 carbon atoms, a substituted or unsubstituted alkylsilyl group having 3 to 20 carbon atoms, a substituted or unsubstituted arylsilyl group having 6 to 20 carbon atoms, a substituted or unsubstituted amino group having 0 to 20 carbon atoms, an acyl group, a carbonyl group, a carboxylic acid group, an ester group, a nitrile group, an isonitrile group, a thiolalkyl group, a sulfinyl group, a sulfonyl group, a phosphino group, and combinations thereof;
adjacent substitutions may be optionally joined to form a ring.

2. The device of claim 1, wherein the first host compound having the structure represent by Formula 4: 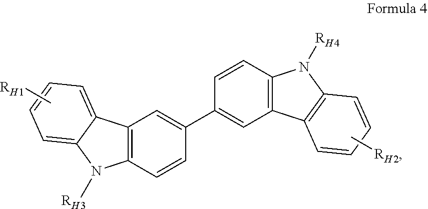
wherein R_{H1} and R_{H2} may represent mono substitution, multiple substitutions or no substitution; when R_{H1}, R_{H2} represent multiple substitutions, each of R_{H1}, R_{H2} may be the same or different;
R_{H1} to R_{H4} are each independently selected from the group consisting of hydrogen, deuterium, halogen, a substituted or unsubstituted alkyl group having 1 to 20 carbon atoms, a substituted or unsubstituted cycloalkyl group having 3 to 20 ring carbon atoms, a substituted or unsubstituted heteroalkyl group having 1 to 20 carbon atoms, a substituted or unsubstituted arylalkyl group having 7 to 30 carbon atoms, a substituted or unsubstituted alkoxy group having 1 to 20 carbon atoms, a substituted or unsubstituted aryloxy group having 6 to 30 carbon atoms, a substituted or unsubstituted alkenyl group having 2 to 20 carbon atoms, a substituted or unsubstituted aryl group having 6 to 30 carbon atoms, a substituted or unsubstituted heteroaryl group having 3 to 30 carbon atoms, a substituted or unsubstituted alkylsilyl group having 3 to 20 carbon atoms, a substituted or unsubstituted arylsilyl group having 6 to 20 carbon atoms, a substituted or unsubstituted amino group having 0 to 20 carbon atoms, an acyl group, a carbonyl group, a carboxylic acid group, an ester group, a nitrile group, an isonitrile group, a thiolalkyl group, a sulfinyl group, a sulfonyl group, a phosphino group, and combinations thereof.

3. The device of claim 1, wherein R_{H1} to R_{H4} are each independently selected from the group consisting of hydrogen, deuterium, a substituted or unsubstituted aryl group having 6 to 30 carbon atoms, a substituted or unsubstituted heteroaryl group having 3 to 30 carbon atoms, and combinations thereof.

4. The device of claim 3, wherein R_{H1} to R_{H4} are each independently selected from the group consisting of hydrogen, deuterium, phenyl, biphenyl, terphenyl, naphthyl, phenanthryl, triphenylene, deuterated phenyl, deuterated biphenyl, deuterated terphenyl, deuterated naphthyl, deuterated phenanthryl, deuterated triphenylene, and combinations thereof.

5. The device of claim 1, wherein the second host compound having the structure represent by Formula 5: 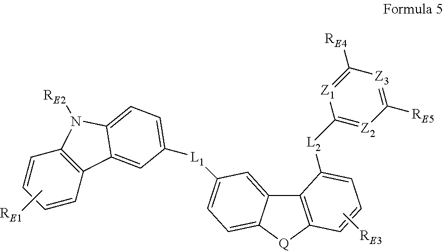
wherein R_{E1} and R_{E3} may represent mono substitution, multiple substitutions or no substitution; when R_{E1}, R_{E3} represent multiple substitutions, each of R_{E1}, R_{E3} may be the same or different;
Q is selected from O or S;
Z₁, Z₂ and Z₃ are each independently selected from CR or N;
L₁ and L₂ are each independently selected from the group consisting of a single bond, a substituted or unsubstituted arylene having 6 to 30 carbon atoms, a substituted or unsubstituted heteroarylene having 3 to 30 carbon atoms, and combinations thereof;
R_{E1} to R_{E5} are each independently selected from the group consisting of hydrogen, deuterium, halogen, a substituted or unsubstituted alkyl group having 1 to 20 carbon atoms, a substituted or unsubstituted cycloalkyl group having 3 to 20 ring carbon atoms, a substituted or unsubstituted heteroalkyl group having 1 to 20 carbon atoms, a substituted or unsubstituted arylalkyl group having 7 to 30 carbon atoms, a substituted or unsubstituted alkoxy group having 1 to 20 carbon atoms, a substituted or unsubstituted aryloxy group having 6 to 30 carbon atoms, a substituted or unsubstituted alkenyl group having 2 to 20 carbon atoms, a substituted or unsubstituted aryl group having 6 to 30 carbon atoms, a substituted or unsubstituted heteroaryl group having 3 to 30 carbon atoms, a substituted or unsubstituted alkylsilyl group having 3 to 20 carbon atoms, a substituted or unsubstituted arylsilyl group having 6 to 20 carbon atoms, a substituted or unsubstituted amino group having 0 to 20 carbon atoms, an acyl group, a carbonyl group, a carboxylic acid group, an ester group, a nitrile group, an isonitrile group, a thiolalkyl group, a sulfinyl group, a sulfonyl group, a phosphino group, and combinations thereof.
adjacent substitutions may be optionally joined to form a ring.

6. The device of claim 1, wherein R_{E1} to R_{E5} are each independently selected from the group consisting of hydrogen, deuterium, a substituted or unsubstituted aryl group having 6 to 30 carbon atoms, a substituted or unsubstituted heteroaryl group having 3 to 30 carbon atoms, and combinations thereof.

7. The device of claim 6, wherein R_{E1} represents multiple substitutions, and adjacent R_{E1} may be optionally joined to form a ring.

8. The device of claim 6, wherein R_{E1} to R_{E5} are each independently selected from the group consisting of hydrogen, deuterium, phenyl, biphenyl, fluorenyl, dibenzofuranyl, naphthyl, deuterated phenyl, deuterated biphenyl, deuterated fluorenyl, deuterated dibenzofuranyl, deuterated naphthyl, and combinations thereof.

9. The device of claim 1, wherein Z₁ to Z₃ are all N.

10. The device of claim 1, wherein L₁ and L₂ are each independently selected from the group consisting of a single bond, a substituted or unsubstituted arylene having 6 to 20 carbon atoms, a substituted or unsubstituted heteroarylene having 3 to 15 carbon atoms, and combinations thereof.

11. The device of claim 10, wherein L₁ and L₂ are each independently selected from a single bond or phenylene.

12. The device of claim 1, wherein X is O.

13. The device of claim 1, wherein Y is CH, CD or N.

14. The device of claim 1, wherein R₁ to R₅ are each independently selected from the group consisting of hydrogen, deuterium, halogen, a substituted or unsubstituted alkyl group having 1 to 20 carbon atoms, a substituted or unsubstituted cycloalkyl group having 3 to 20 ring carbon atoms, and combinations thereof.

15. The device of claim 14, wherein R₁ to R₅ are each independently selected from the group consisting of hydrogen, deuterium, fluorine, methyl, isopropyl, deuterated methyl, deuterated isopropyl, and combinations thereof.

16. The device of claim 1, wherein the first host compound is selected from the group consisting of:

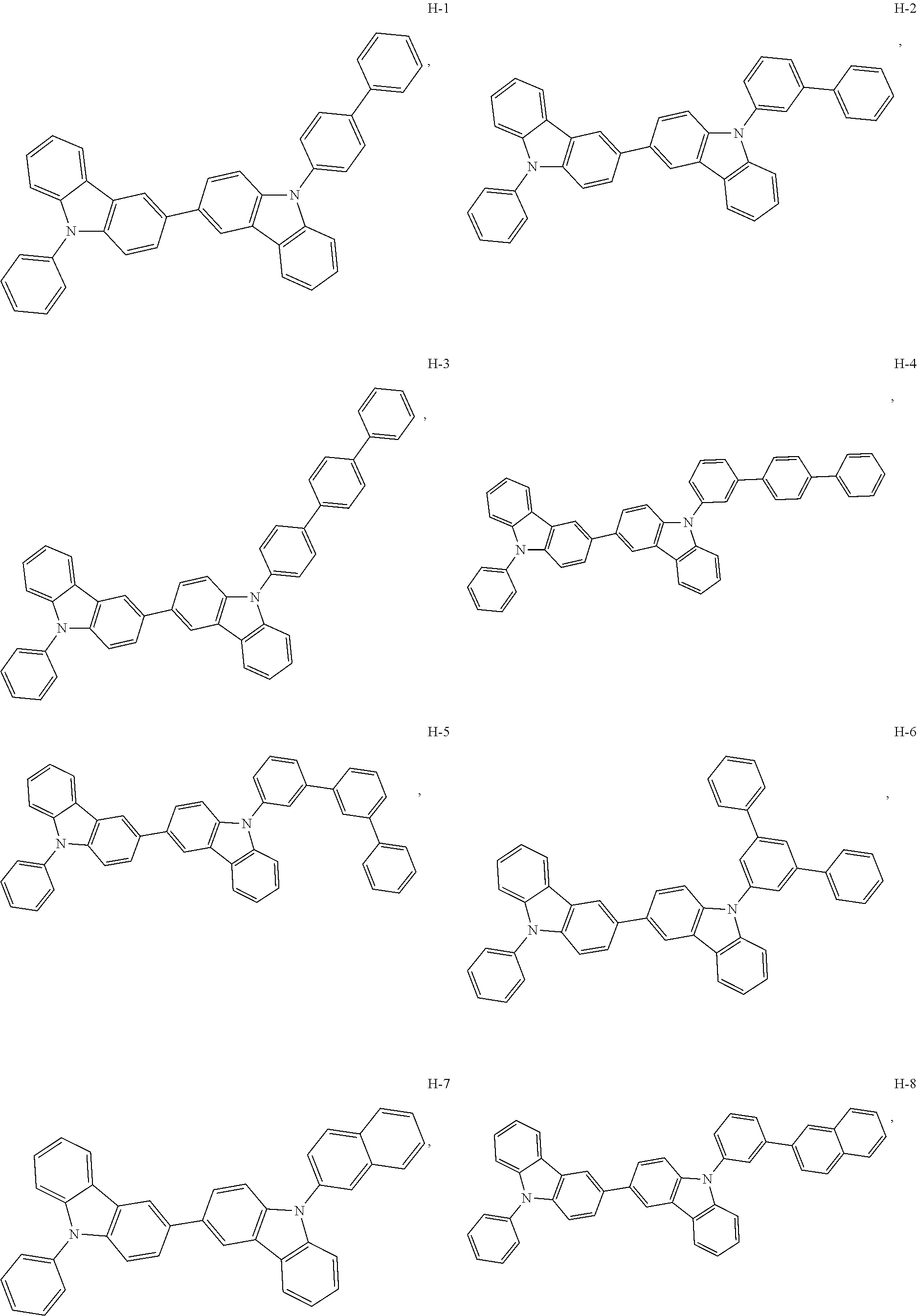

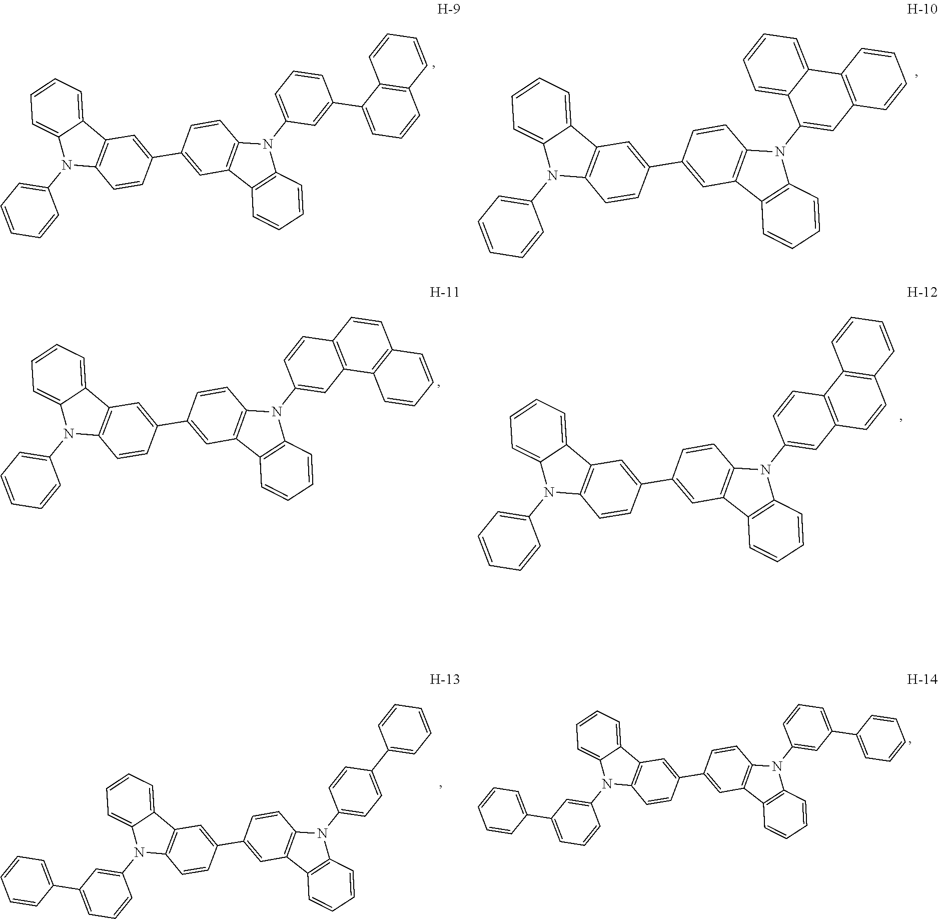 [SMILES: c1ccc(-n2c3ccccc3c3cc(-c4ccc5c(c4)c4ccccc4n5-c4cccc(-c5cccc6ccccc56)c4)ccc32)cc1 ; c1ccc(-n2c3ccccc3c3cc(-c4ccc5c(c4)c4ccccc4n5-c4cc5ccccc5c5ccccc45)ccc32)cc1 ; c1ccc(-n2c3ccccc3c3cc(-c4ccc5c(c4)c4ccccc4n5-c4ccc5ccc6ccccc6c5c4)ccc32)cc1 ; c1ccc(-n2c3ccccc3c3cc(-c4ccc5c(c4)c4ccccc4n5-c4ccc5c(ccc6ccccc65)c4)ccc32)cc1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1]

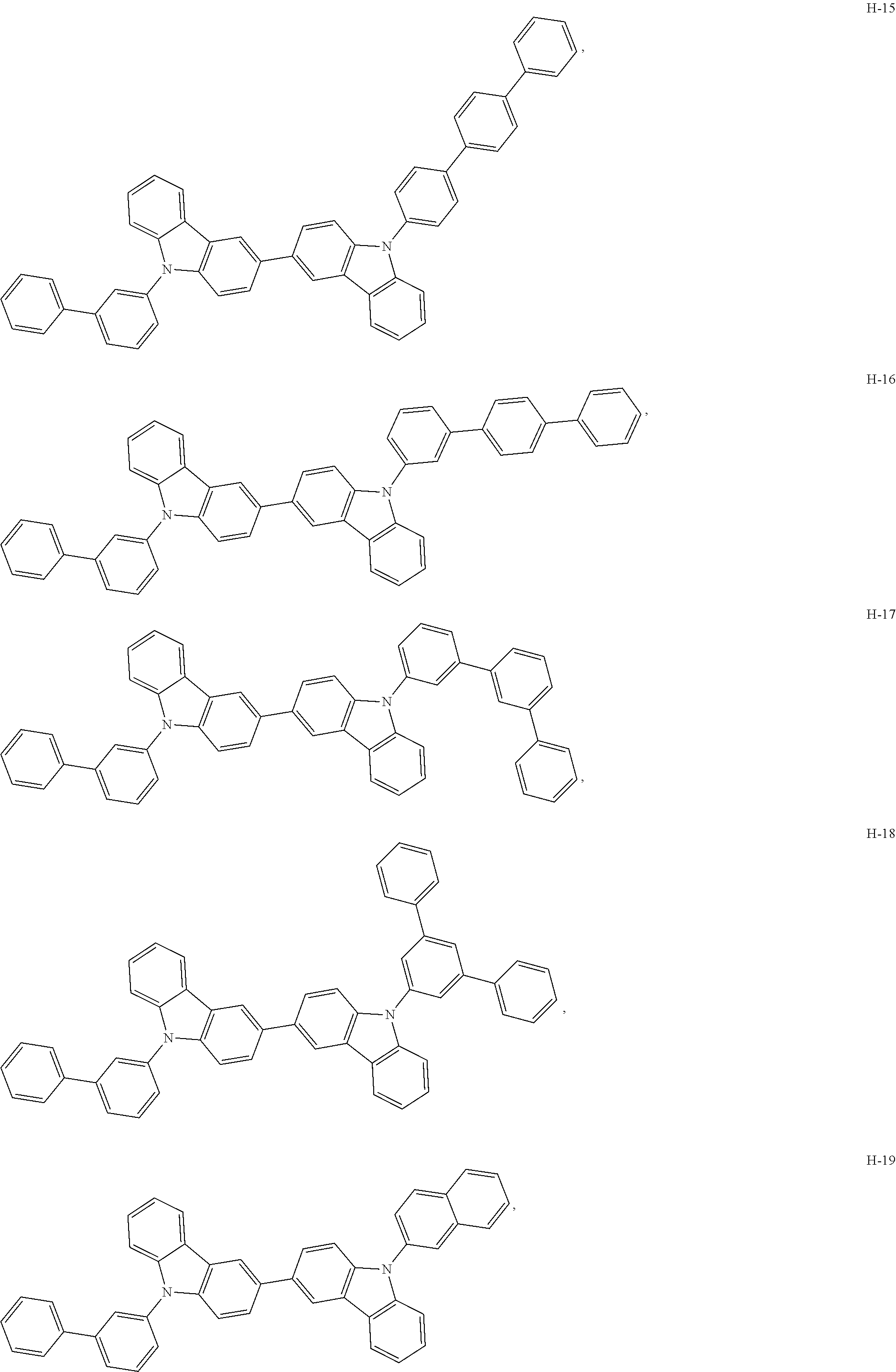

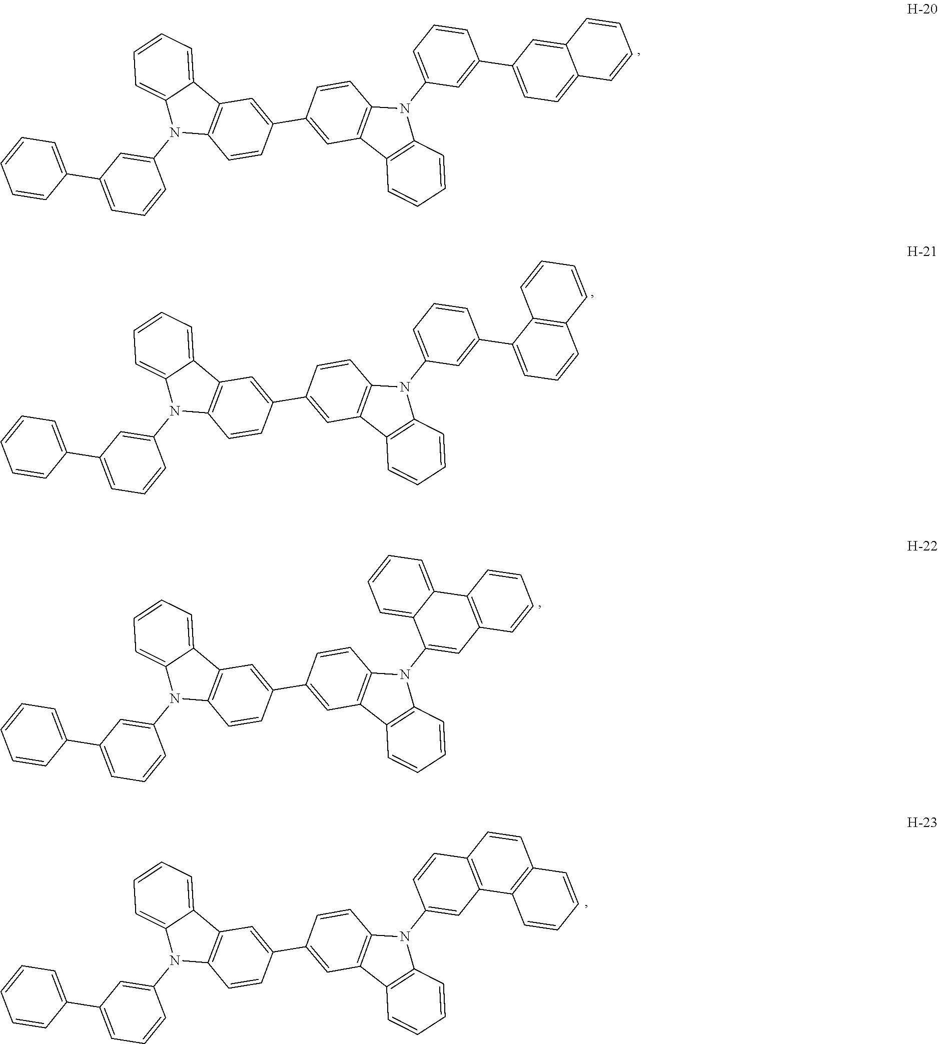 [SMILES: c1ccc(-c2cccc(-n3c4ccccc4c4cc(-c5ccc6c(c5)c5ccccc5n6-c5cccc(-c6ccc7ccccc7c6)c5)ccc43)c2)cc1 ; c1ccc(-c2cccc(-n3c4ccccc4c4cc(-c5ccc6c(c5)c5ccccc5n6-c5cccc(-c6cccc7ccccc67)c5)ccc43)c2)cc1 ; c1ccc(-c2cccc(-n3c4ccccc4c4cc(-c5ccc6c(c5)c5ccccc5n6-c5cc6ccccc6c6ccccc56)ccc43)c2)cc1 ; c1ccc(-c2cccc(-n3c4ccccc4c4cc(-c5ccc6c(c5)c5ccccc5n6-c5ccc6ccc7ccccc7c6c5)ccc43)c2)cc1]

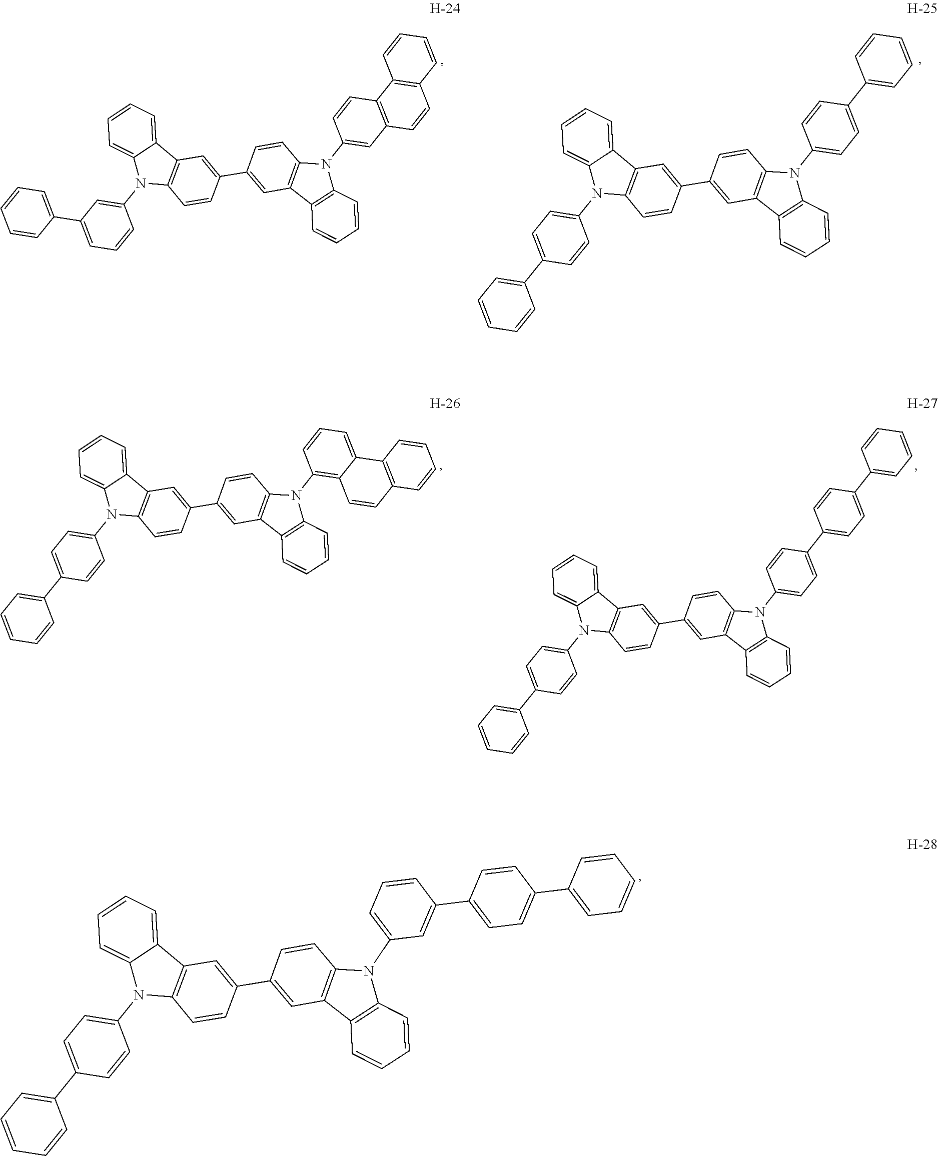 [SMILES: c1ccc(-c2cccc(-n3c4ccccc4c4cc(-c5ccc6c(c5)c5ccccc5n6-c5ccc6c(ccc7ccccc76)c5)ccc43)c2)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-n3c4ccccc4c4cc(-c5ccc6c(c5)c5ccccc5n6-c5cccc6c5ccc5ccccc56)ccc43)cc2)cc1 ; c1ccc(-c2ccc(-c3ccc(-n4c5ccccc5c5cc(-c6ccc7c(c6)c6ccccc6n7-c6ccc(-c7ccccc7)cc6)ccc54)cc3)cc2)cc1 ; c1ccc(-c2ccc(-c3cccc(-n4c5ccccc5c5cc(-c6ccc7c(c6)c6ccccc6n7-c6ccc(-c7ccccc7)cc6)ccc54)c3)cc2)cc1]

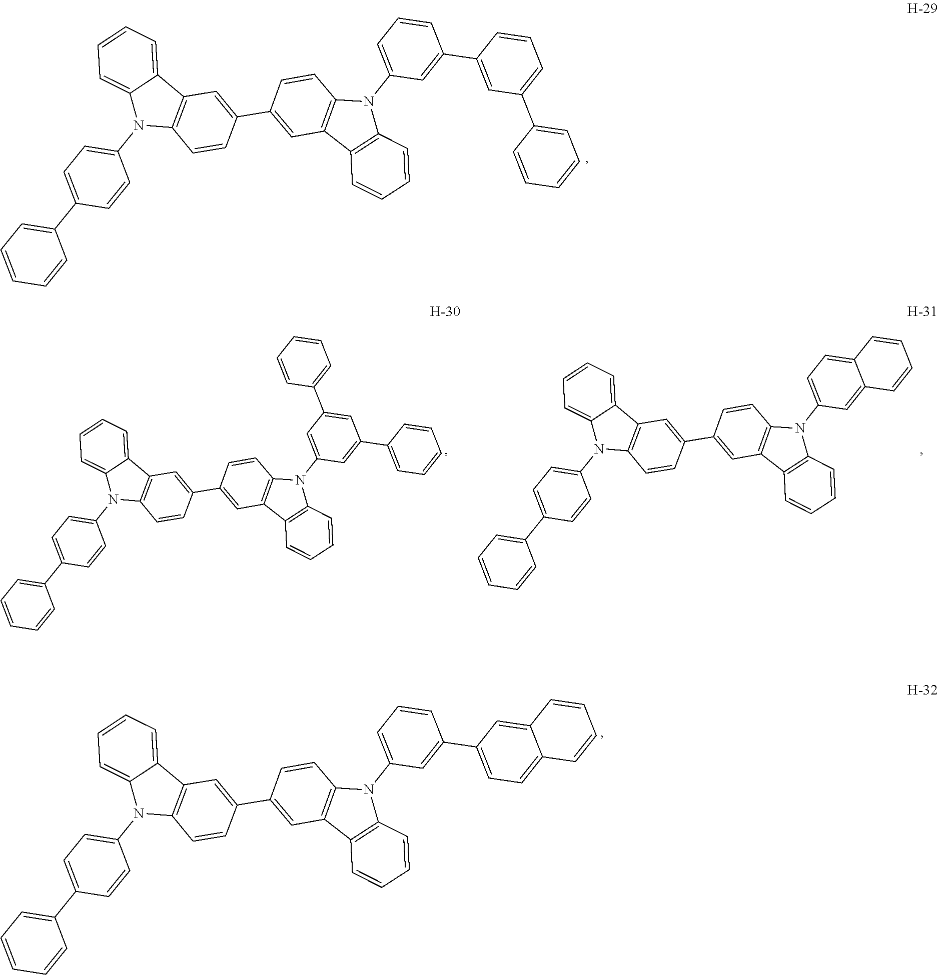 [SMILES: c1ccc(-c2ccc(-n3c4ccccc4c4cc(-c5ccc6c(c5)c5ccccc5n6-c5cccc(-c6cccc(-c7ccccc7)c6)c5)ccc43)cc2)cc1 ; c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1 ; c1ccc(-c2ccc(-n3c4ccccc4c4cc(-c5ccc6c(c5)c5ccccc5n6-c5ccc6ccccc6c5)ccc43)cc2)cc1 ; c1ccc(-c2ccc(-n3c4ccccc4c4cc(-c5ccc6c(c5)c5ccccc5n6-c5cccc(-c6ccc7ccccc7c6)c5)ccc43)cc2)cc1]

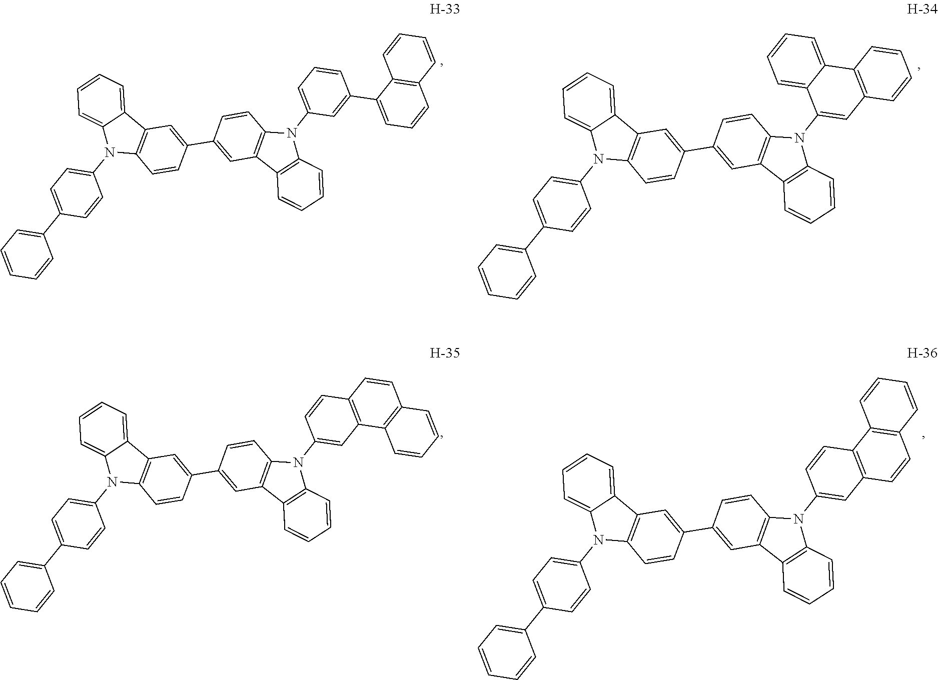 [SMILES: c1ccc(-c2ccc(-n3c4ccccc4c4cc(-c5ccc6c(c5)c5ccccc5n6-c5cccc(-c6cccc7ccccc67)c5)ccc43)cc2)cc1 ; c1ccc(-c2ccc(-n3c4ccccc4c4cc(-c5ccc6c(c5)c5ccccc5n6-c5cc6ccccc6c6ccccc56)ccc43)cc2)cc1 ; c1ccc(-c2ccc(-n3c4ccccc4c4cc(-c5ccc6c(c5)c5ccccc5n6-c5ccc6ccc7ccccc7c6c5)ccc43)cc2)cc1 ; c1ccc(-c2ccc(-n3c4ccccc4c4cc(-c5ccc6c(c5)c5ccccc5n6-c5ccc6c(ccc7ccccc76)c5)ccc43)cc2)cc1]

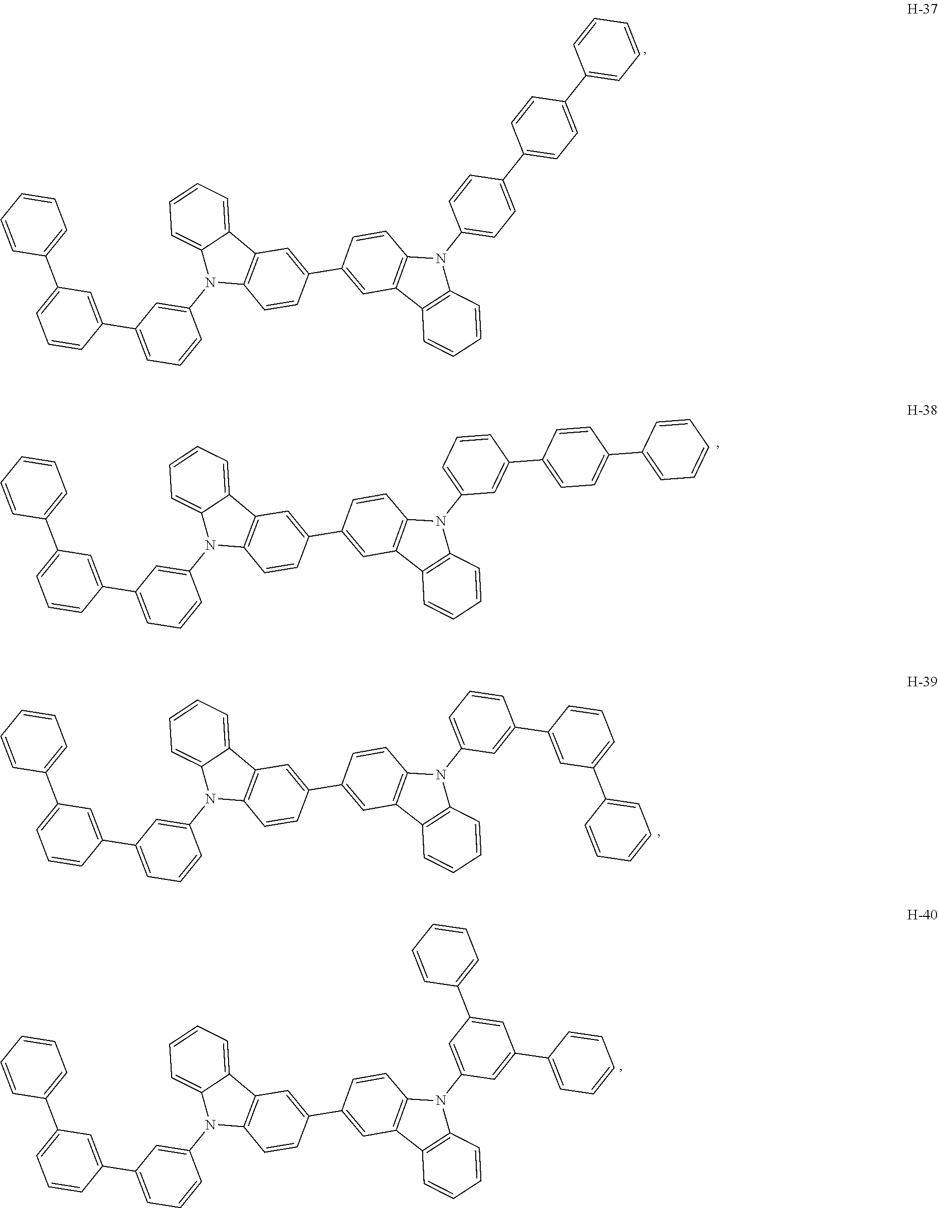 [SMILES: c1ccc(-c2ccc(-c3ccc(-n4c5ccccc5c5cc(-c6ccc7c(c6)c6ccccc6n7-c6cccc(-c7cccc(-c8ccccc8)c7)c6)ccc54)cc3)cc2)cc1 ; c1ccc(-c2ccc(-c3cccc(-n4c5ccccc5c5cc(-c6ccc7c(c6)c6ccccc6n7-c6cccc(-c7cccc(-c8ccccc8)c7)c6)ccc54)c3)cc2)cc1 ; c1ccc(-c2cccc(-c3cccc(-n4c5ccccc5c5cc(-c6ccc7c(c6)c6ccccc6n7-c6cccc(-c7cccc(-c8ccccc8)c7)c6)ccc54)c3)c2)cc1 ; c1ccc(-c2cccc(-c3cccc(-n4c5ccccc5c5cc(-c6ccc7c(c6)c6ccccc6n7-c6cc(-c7ccccc7)cc(-c7ccccc7)c6)ccc54)c3)c2)cc1]

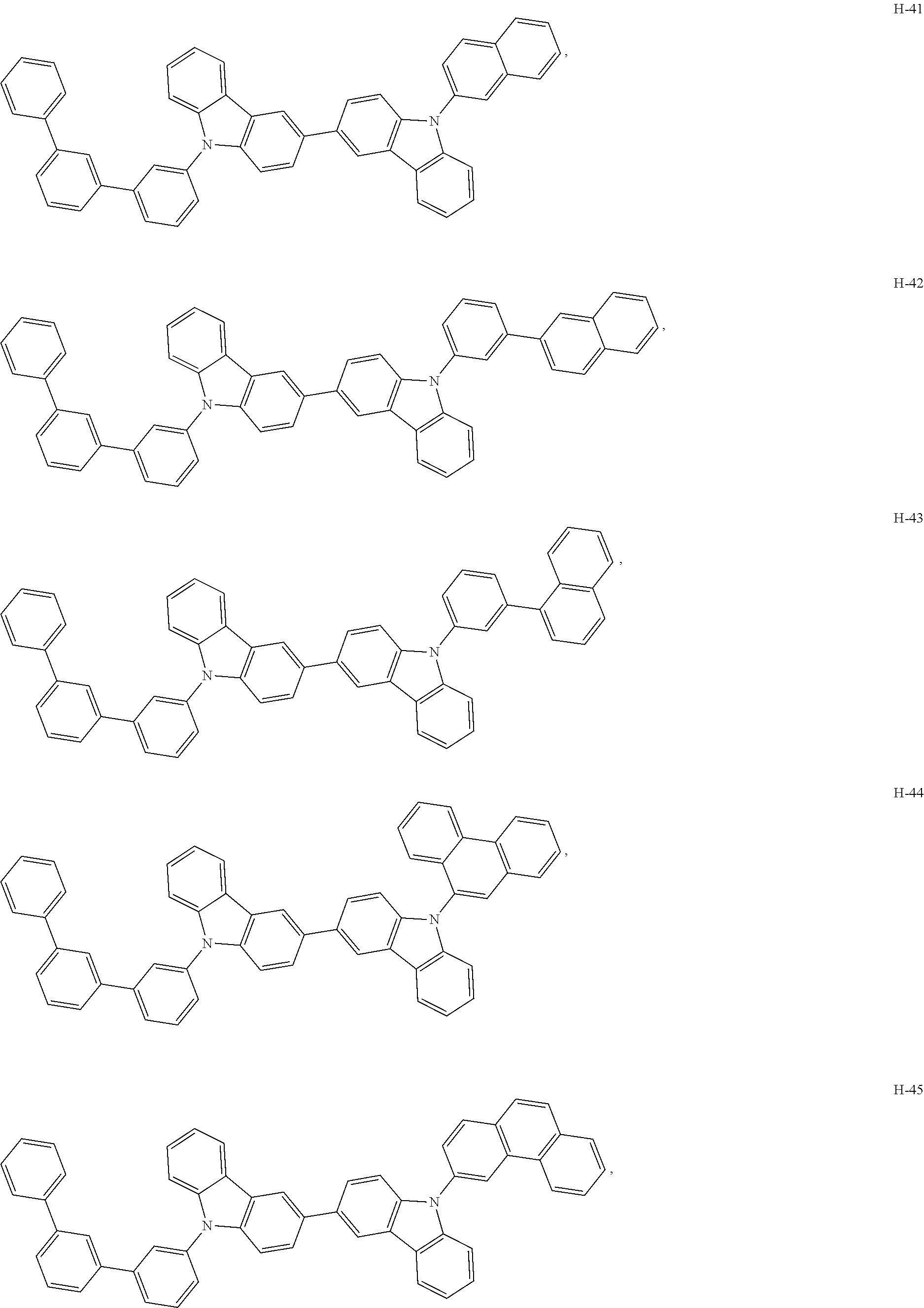

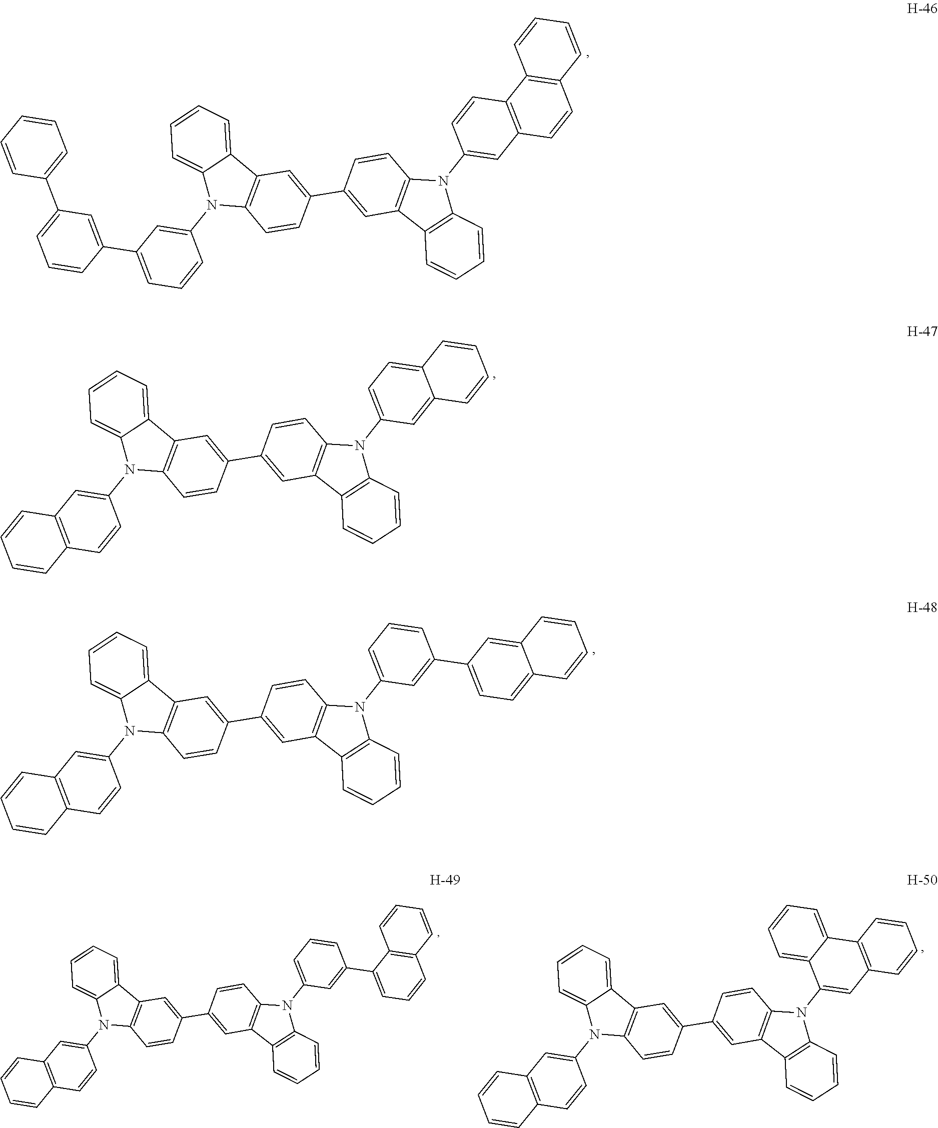 [SMILES: c1ccc(-c2cccc(-c3cccc(-n4c5ccccc5c5cc(-c6ccc7c(c6)c6ccccc6n7-c6ccc7c(ccc8ccccc87)c6)ccc54)c3)c2)cc1 ; c1ccc2cc(-n3c4ccccc4c4cc(-c5ccc6c(c5)c5ccccc5n6-c5ccc6ccccc6c5)ccc43)ccc2c1 ; c1cc(-c2ccc3ccccc3c2)cc(-n2c3ccccc3c3cc(-c4ccc5c(c4)c4ccccc4n5-c4ccc5ccccc5c4)ccc32)c1 ; c1cc(-c2cccc3ccccc23)cc(-n2c3ccccc3c3cc(-c4ccc5c(c4)c4ccccc4n5-c4ccc5ccccc5c4)ccc32)c1 ; c1ccc2cc(-n3c4ccccc4c4cc(-c5ccc6c(c5)c5ccccc5n6-c5cc6ccccc6c6ccccc56)ccc43)ccc2c1]

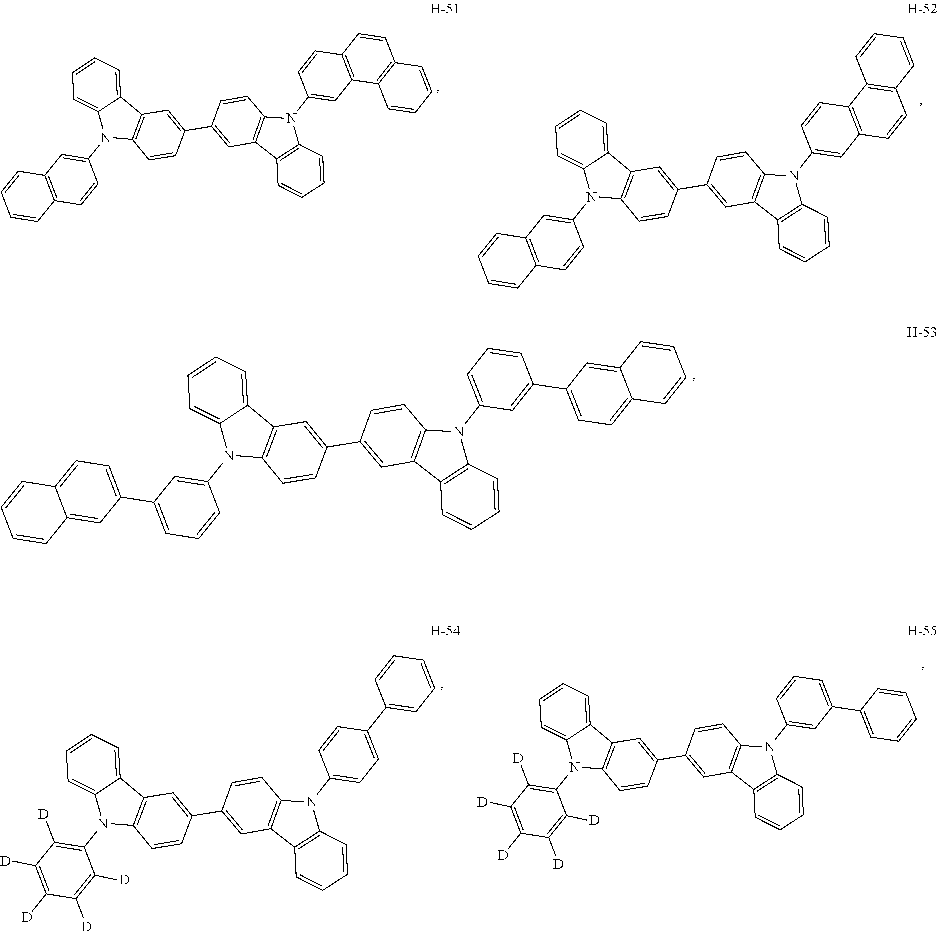 [SMILES: c1ccc2cc(-n3c4ccccc4c4cc(-c5ccc6c(c5)c5ccccc5n6-c5ccc6ccc7ccccc7c6c5)ccc43)ccc2c1 ; c1ccc2cc(-n3c4ccccc4c4cc(-c5ccc6c(c5)c5ccccc5n6-c5ccc6c(ccc7ccccc76)c5)ccc43)ccc2c1 ; c1cc(-c2ccc3ccccc3c2)cc(-n2c3ccccc3c3cc(-c4ccc5c(c4)c4ccccc4n5-c4cccc(-c5ccc6ccccc6c5)c4)ccc32)c1 ; [2H]c1c([2H])c([2H])c(-n2c3ccccc3c3cc(-c4ccc5c(c4)c4ccccc4n5-c4ccc(-c5ccccc5)cc4)ccc32)c([2H])c1[2H] ; [2H]c1c([2H])c([2H])c(-n2c3ccccc3c3cc(-c4ccc5c(c4)c4ccccc4n5-c4cccc(-c5ccccc5)c4)ccc32)c([2H])c1[2H]]

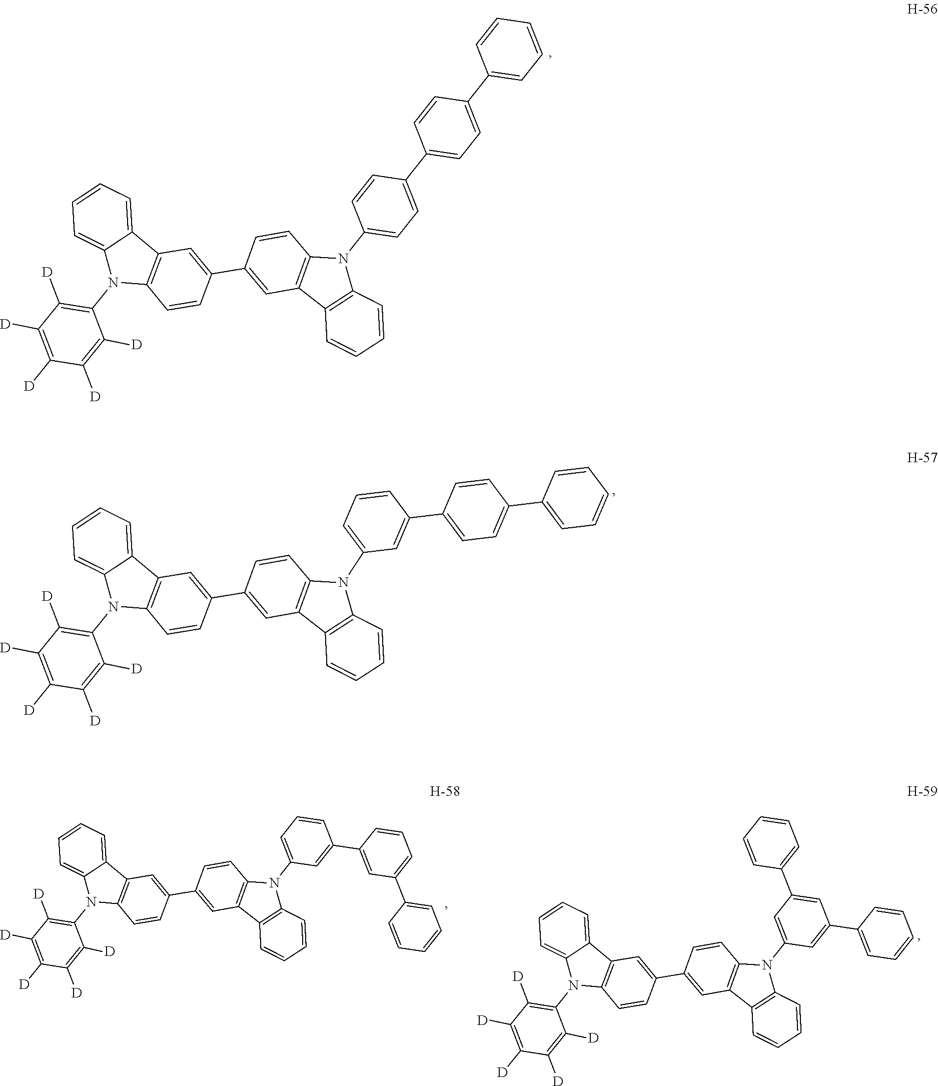 [SMILES: [2H]c1c([2H])c([2H])c(-n2c3ccccc3c3cc(-c4ccc5c(c4)c4ccccc4n5-c4ccc(-c5ccc(-c6ccccc6)cc5)cc4)ccc32)c([2H])c1[2H] ; [2H]c1c([2H])c([2H])c(-n2c3ccccc3c3cc(-c4ccc5c(c4)c4ccccc4n5-c4cccc(-c5ccc(-c6ccccc6)cc5)c4)ccc32)c([2H])c1[2H] ; [2H]c1c([2H])c([2H])c(-n2c3ccccc3c3cc(-c4ccc5c(c4)c4ccccc4n5-c4cccc(-c5cccc(-c6ccccc6)c5)c4)ccc32)c([2H])c1[2H] ; [2H]c1c([2H])c([2H])c(-n2c3ccccc3c3cc(-c4ccc5c(c4)c4ccccc4n5-c4cc(-c5ccccc5)cc(-c5ccccc5)c4)ccc32)c([2H])c1[2H]]

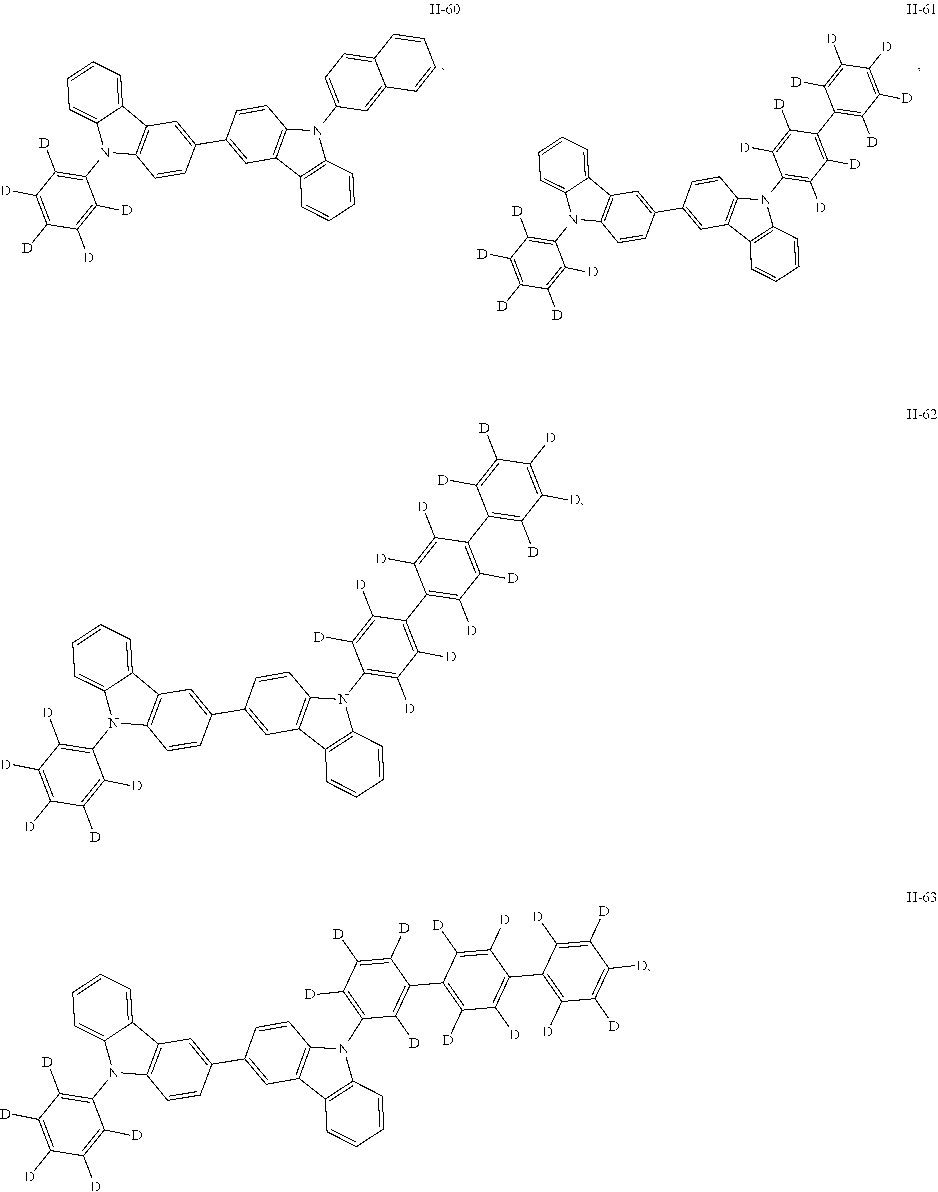

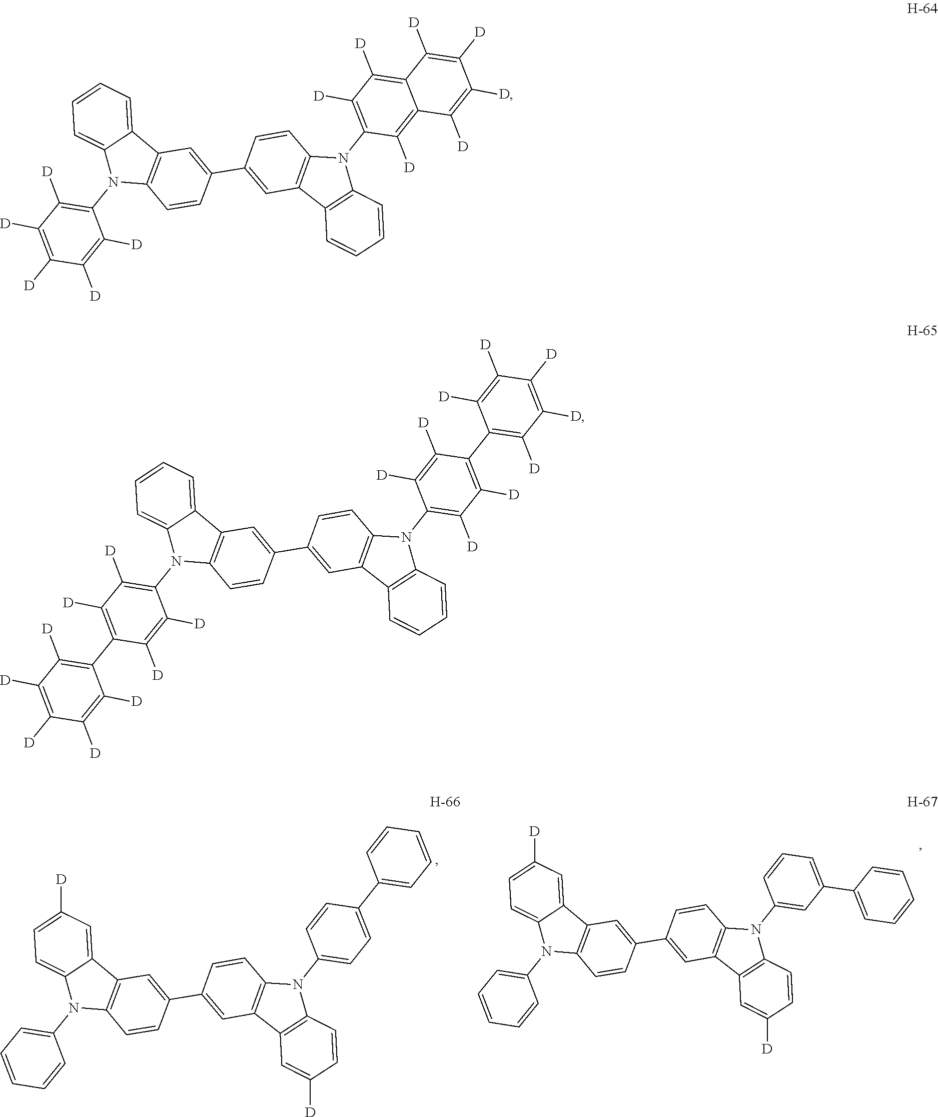

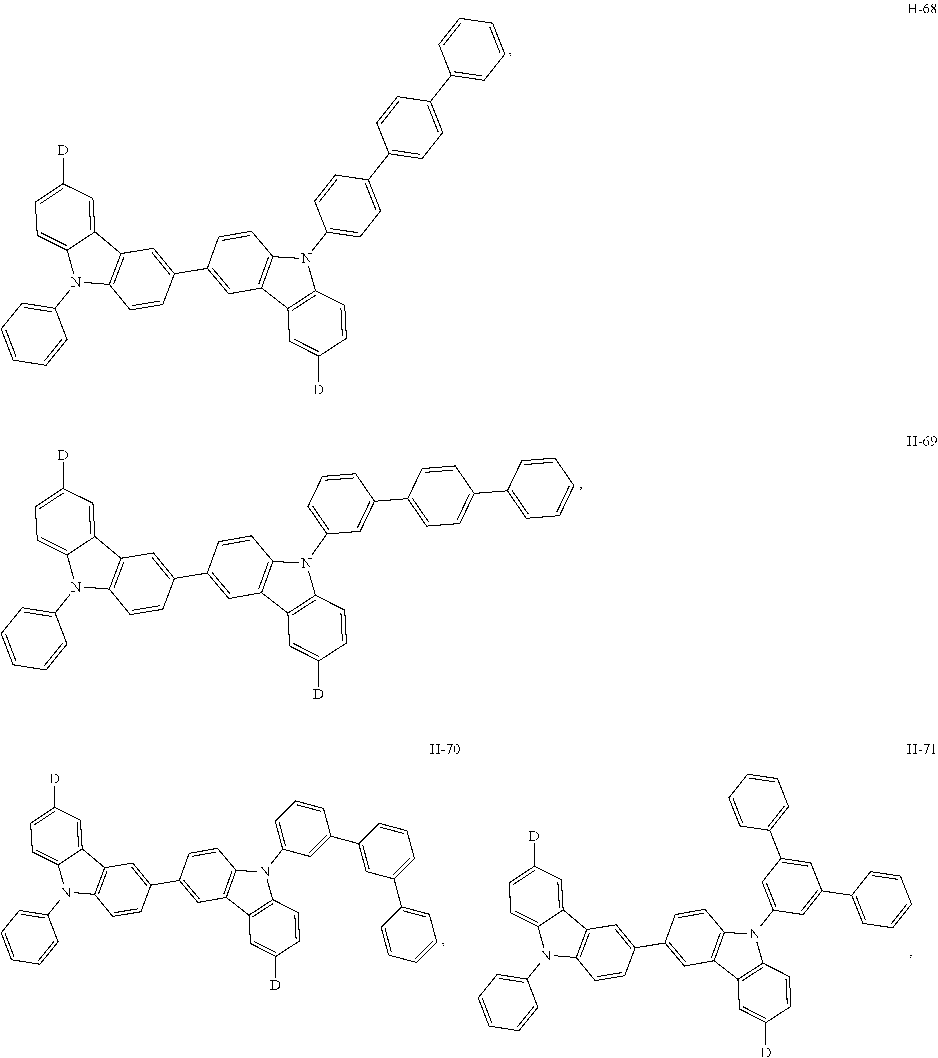 [SMILES: [2H]c1ccc2c(c1)c1cc(-c3ccc4c(c3)c3cc([2H])ccc3n4-c3ccc(-c4ccc(-c5ccccc5)cc4)cc3)ccc1n2-c1ccccc1 ; [2H]c1ccc2c(c1)c1cc(-c3ccc4c(c3)c3cc([2H])ccc3n4-c3cccc(-c4ccc(-c5ccccc5)cc4)c3)ccc1n2-c1ccccc1 ; [2H]c1ccc2c(c1)c1cc(-c3ccc4c(c3)c3cc([2H])ccc3n4-c3cccc(-c4cccc(-c5ccccc5)c4)c3)ccc1n2-c1ccccc1 ; [2H]c1ccc2c(c1)c1cc(-c3ccc4c(c3)c3cc([2H])ccc3n4-c3cc(-c4ccccc4)cc(-c4ccccc4)c3)ccc1n2-c1ccccc1]

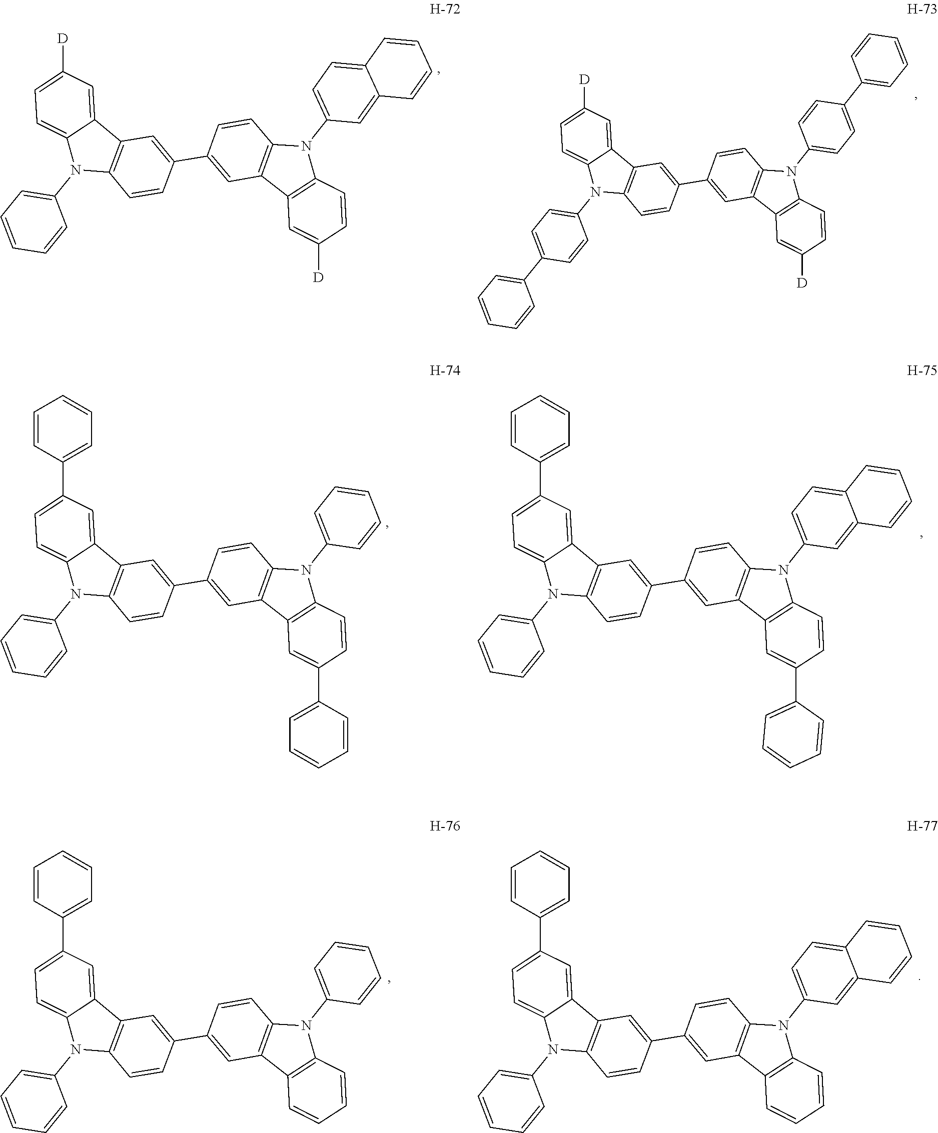

17. The device of claim 1, wherein the second host compound is selected from the group consisting of:

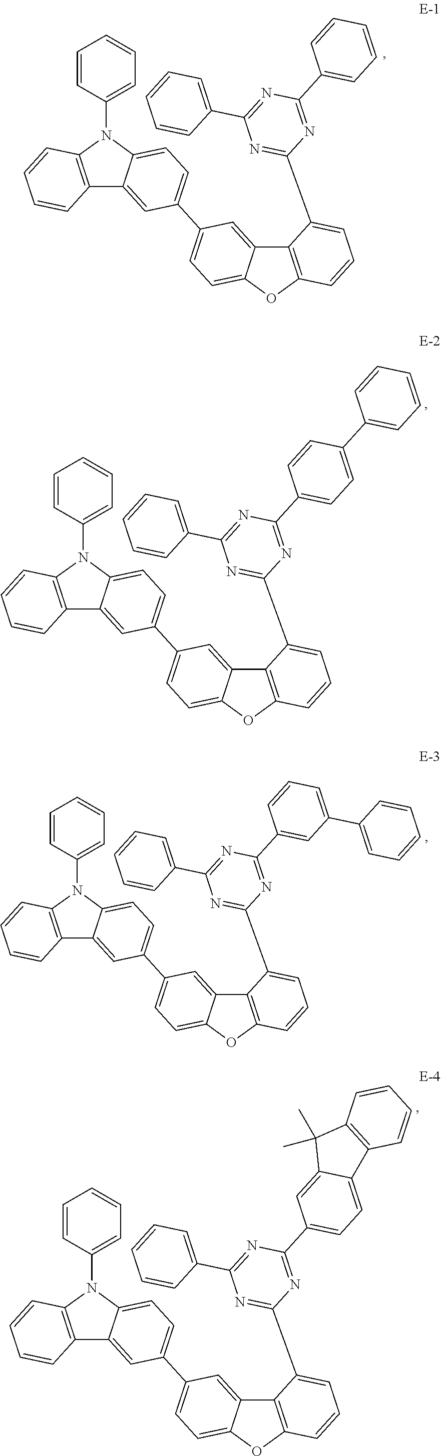 [SMILES: c1ccc(-c2nc(-c3ccccc3)nc(-c3cccc4oc5ccc(-c6ccc7c(c6)c6ccccc6n7-c6ccccc6)cc5c34)n2)cc1 ; c1ccc(-c2ccc(-c3nc(-c4ccccc4)nc(-c4cccc5oc6ccc(-c7ccc8c(c7)c7ccccc7n8-c7ccccc7)cc6c45)n3)cc2)cc1 ; c1ccc(-c2cccc(-c3nc(-c4ccccc4)nc(-c4cccc5oc6ccc(-c7ccc8c(c7)c7ccccc7n8-c7ccccc7)cc6c45)n3)c2)cc1 ; CC1(C)c2ccccc2-c2ccc(-c3nc(-c4ccccc4)nc(-c4cccc5oc6ccc(-c7ccc8c(c7)c7ccccc7n8-c7ccccc7)cc6c45)n3)cc21]

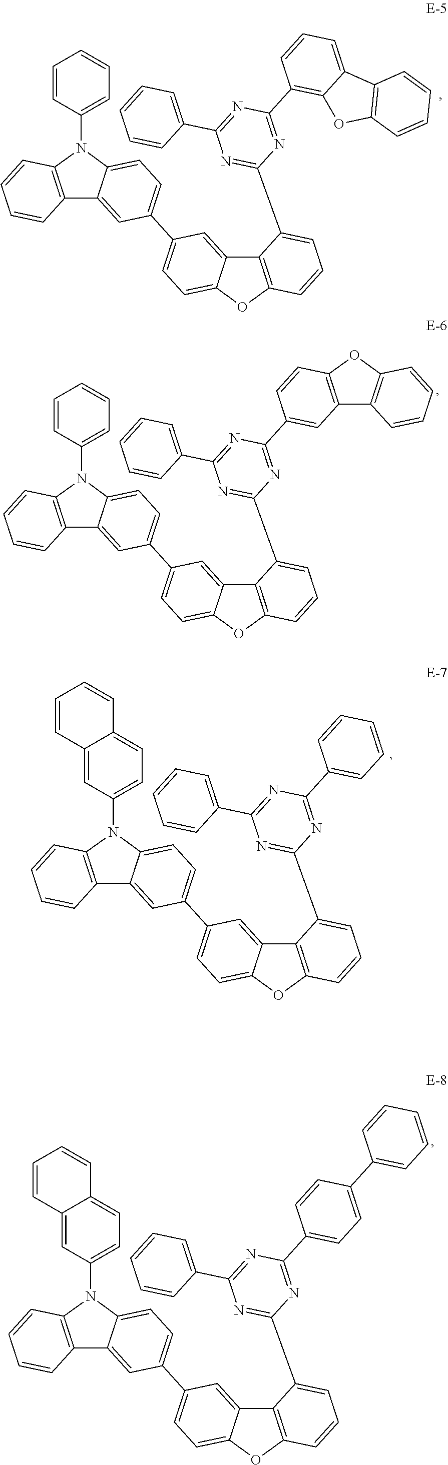 [SMILES: c1ccc(-c2nc(-c3cccc4c3oc3ccccc34)nc(-c3cccc4oc5ccc(-c6ccc7c(c6)c6ccccc6n7-c6ccccc6)cc5c34)n2)cc1 ; c1ccc(-c2nc(-c3ccc4oc5ccccc5c4c3)nc(-c3cccc4oc5ccc(-c6ccc7c(c6)c6ccccc6n7-c6ccccc6)cc5c34)n2)cc1 ; c1ccc(-c2nc(-c3ccccc3)nc(-c3cccc4oc5ccc(-c6ccc7c(c6)c6ccccc6n7-c6ccc7ccccc7c6)cc5c34)n2)cc1 ; c1ccc(-c2ccc(-c3nc(-c4ccccc4)nc(-c4cccc5oc6ccc(-c7ccc8c(c7)c7ccccc7n8-c7ccc8ccccc8c7)cc6c45)n3)cc2)cc1]

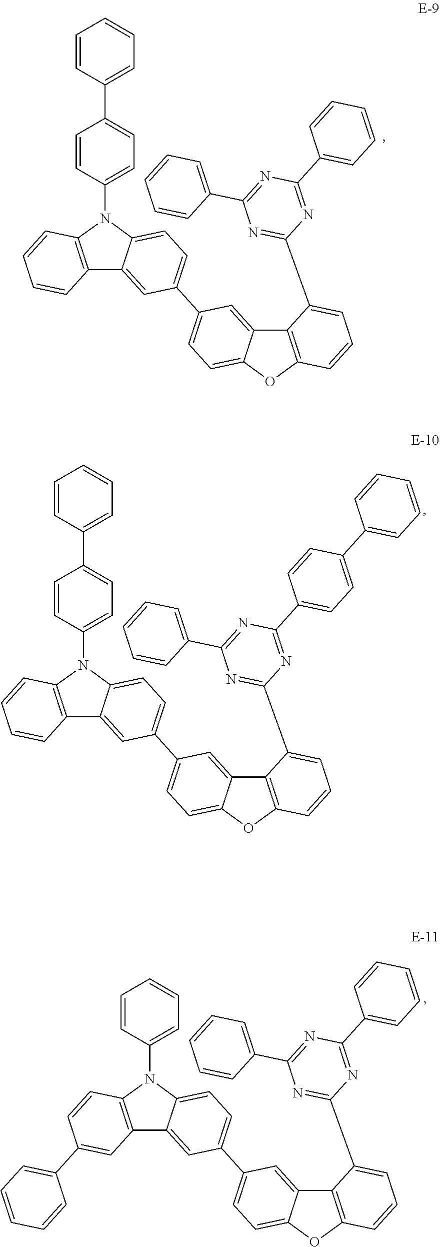 [SMILES: c1ccc(-c2ccc(-n3c4ccccc4c4cc(-c5ccc6oc7cccc(-c8nc(-c9ccccc9)nc(-c9ccccc9)n8)c7c6c5)ccc43)cc2)cc1 ; c1ccc(-c2ccc(-c3nc(-c4ccccc4)nc(-c4cccc5oc6ccc(-c7ccc8c(c7)c7ccccc7n8-c7ccc(-c8ccccc8)cc7)cc6c45)n3)cc2)cc1 ; c1ccc(-c2ccc3c(c2)c2cc(-c4ccc5oc6cccc(-c7nc(-c8ccccc8)nc(-c8ccccc8)n7)c6c5c4)ccc2n3-c2ccccc2)cc1]

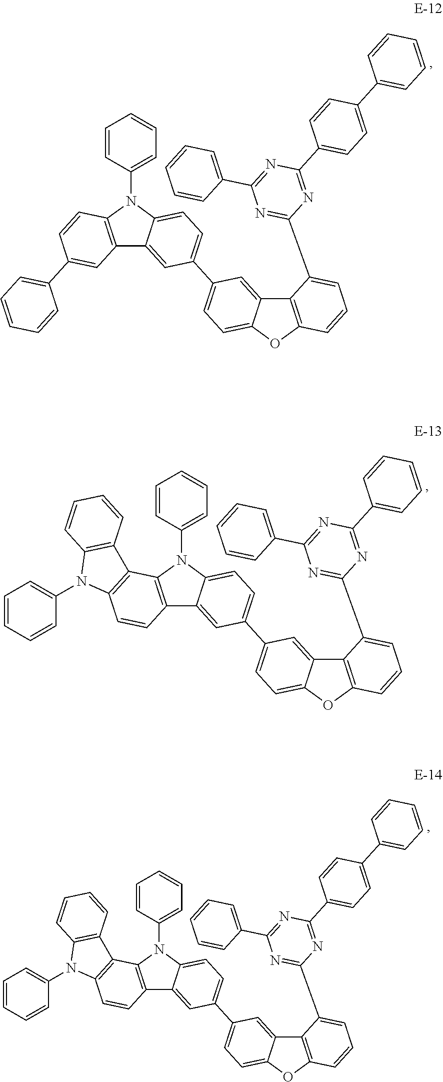 [SMILES: c1ccc(-c2ccc(-c3nc(-c4ccccc4)nc(-c4cccc5oc6ccc(-c7ccc8c(c7)c7cc(-c9ccccc9)ccc7n8-c7ccccc7)cc6c45)n3)cc2)cc1 ; c1ccc(-c2nc(-c3ccccc3)nc(-c3cccc4oc5ccc(-c6ccc7c(c6)c6ccc8c(c9ccccc9n8-c8ccccc8)c6n7-c6ccccc6)cc5c34)n2)cc1 ; c1ccc(-c2ccc(-c3nc(-c4ccccc4)nc(-c4cccc5oc6ccc(-c7ccc8c(c7)c7ccc9c(c%10ccccc%10n9-c9ccccc9)c7n8-c7ccccc7)cc6c45)n3)cc2)cc1]

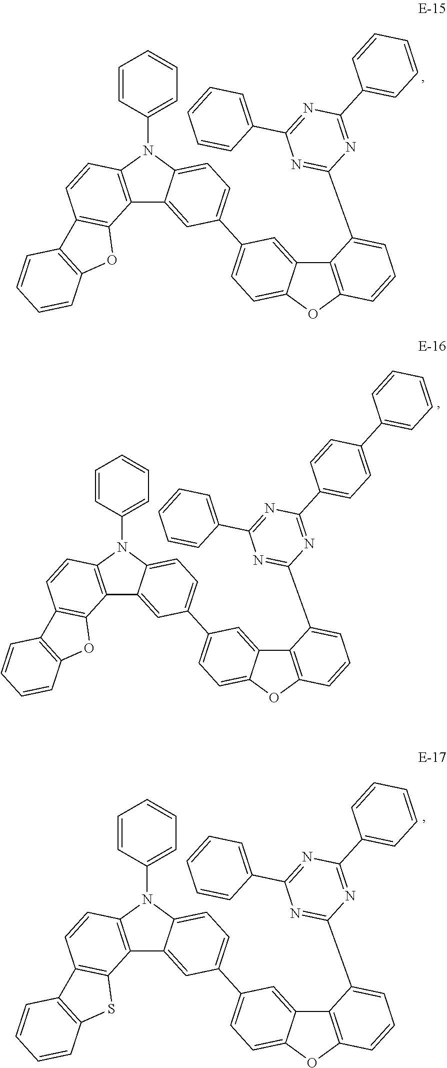 [SMILES: c1ccc(-c2nc(-c3ccccc3)nc(-c3cccc4oc5ccc(-c6ccc7c(c6)c6c8oc9ccccc9c8ccc6n7-c6ccccc6)cc5c34)n2)cc1 ; c1ccc(-c2ccc(-c3nc(-c4ccccc4)nc(-c4cccc5oc6ccc(-c7ccc8c(c7)c7c9oc%10ccccc%10c9ccc7n8-c7ccccc7)cc6c45)n3)cc2)cc1 ; c1ccc(-c2nc(-c3ccccc3)nc(-c3cccc4oc5ccc(-c6ccc7c(c6)c6c8sc9ccccc9c8ccc6n7-c6ccccc6)cc5c34)n2)cc1]

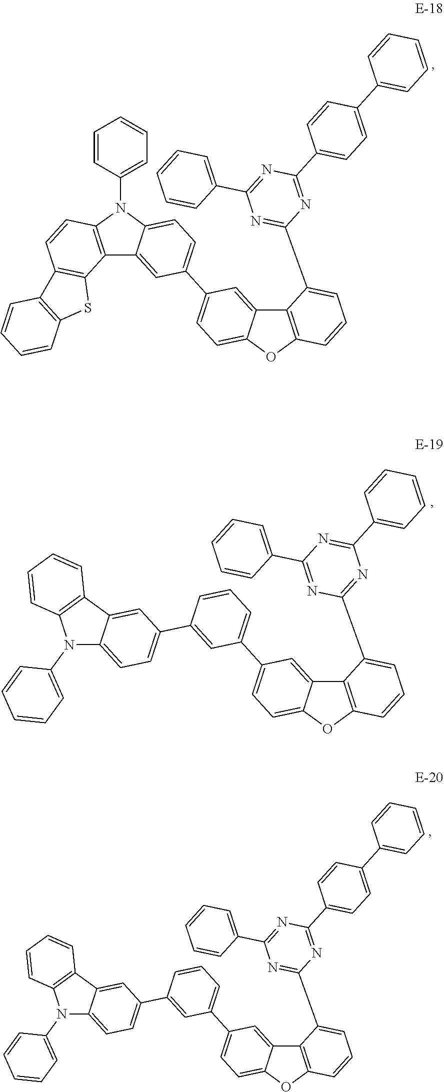 [SMILES: c1ccc(-c2ccc(-c3nc(-c4ccccc4)nc(-c4cccc5oc6ccc(-c7ccc8c(c7)c7c9sc%10ccccc%10c9ccc7n8-c7ccccc7)cc6c45)n3)cc2)cc1 ; c1ccc(-c2nc(-c3ccccc3)nc(-c3cccc4oc5ccc(-c6cccc(-c7ccc8c(c7)c7ccccc7n8-c7ccccc7)c6)cc5c34)n2)cc1 ; c1ccc(-c2ccc(-c3nc(-c4ccccc4)nc(-c4cccc5oc6ccc(-c7cccc(-c8ccc9c(c8)c8ccccc8n9-c8ccccc8)c7)cc6c45)n3)cc2)cc1]

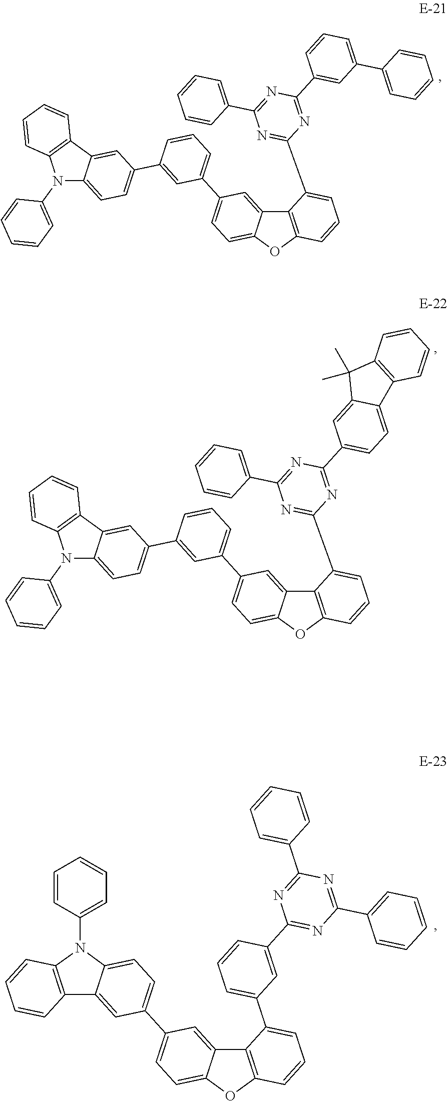 [SMILES: c1ccc(-c2cccc(-c3nc(-c4ccccc4)nc(-c4cccc5oc6ccc(-c7cccc(-c8ccc9c(c8)c8ccccc8n9-c8ccccc8)c7)cc6c45)n3)c2)cc1 ; CC1(C)c2ccccc2-c2ccc(-c3nc(-c4ccccc4)nc(-c4cccc5oc6ccc(-c7cccc(-c8ccc9c(c8)c8ccccc8n9-c8ccccc8)c7)cc6c45)n3)cc21 ; c1ccc(-c2nc(-c3ccccc3)nc(-c3cccc(-c4cccc5oc6ccc(-c7ccc8c(c7)c7ccccc7n8-c7ccccc7)cc6c45)c3)n2)cc1]

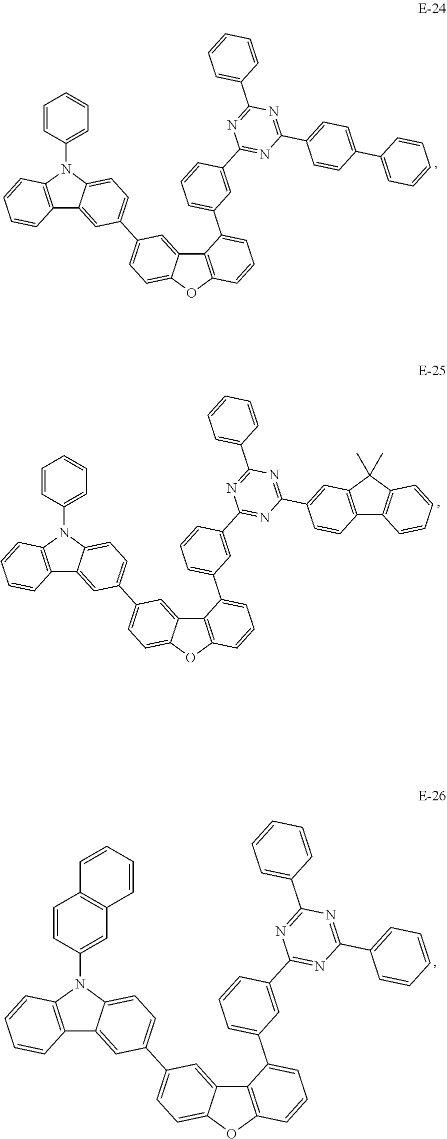 [SMILES: c1ccc(-c2ccc(-c3nc(-c4ccccc4)nc(-c4cccc(-c5cccc6oc7ccc(-c8ccc9c(c8)c8ccccc8n9-c8ccccc8)cc7c56)c4)n3)cc2)cc1 ; CC1(C)c2ccccc2-c2ccc(-c3nc(-c4ccccc4)nc(-c4cccc(-c5cccc6oc7ccc(-c8ccc9c(c8)c8ccccc8n9-c8ccccc8)cc7c56)c4)n3)cc21 ; c1ccc(-c2nc(-c3ccccc3)nc(-c3cccc(-c4cccc5oc6ccc(-c7ccc8c(c7)c7ccccc7n8-c7ccc8ccccc8c7)cc6c45)c3)n2)cc1]

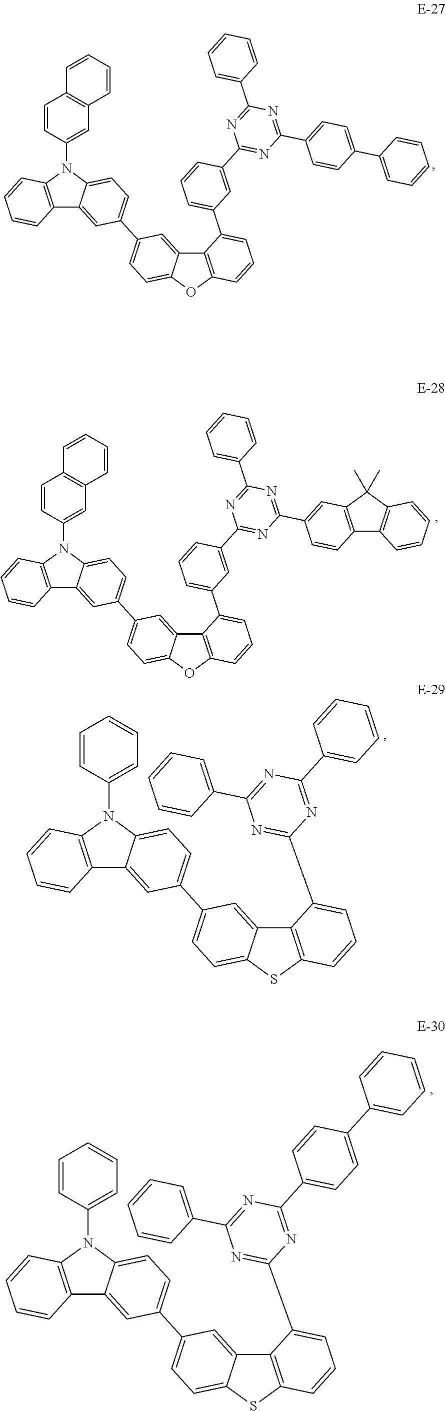 [SMILES: c1ccc(-c2ccc(-c3nc(-c4ccccc4)nc(-c4cccc(-c5cccc6oc7ccc(-c8ccc9c(c8)c8ccccc8n9-c8ccc9ccccc9c8)cc7c56)c4)n3)cc2)cc1 ; CC1(C)c2ccccc2-c2ccc(-c3nc(-c4ccccc4)nc(-c4cccc(-c5cccc6oc7ccc(-c8ccc9c(c8)c8ccccc8n9-c8ccc9ccccc9c8)cc7c56)c4)n3)cc21 ; c1ccc(-c2nc(-c3ccccc3)nc(-c3cccc4sc5ccc(-c6ccc7c(c6)c6ccccc6n7-c6ccccc6)cc5c34)n2)cc1 ; c1ccc(-c2ccc(-c3nc(-c4ccccc4)nc(-c4cccc5sc6ccc(-c7ccc8c(c7)c7ccccc7n8-c7ccccc7)cc6c45)n3)cc2)cc1]

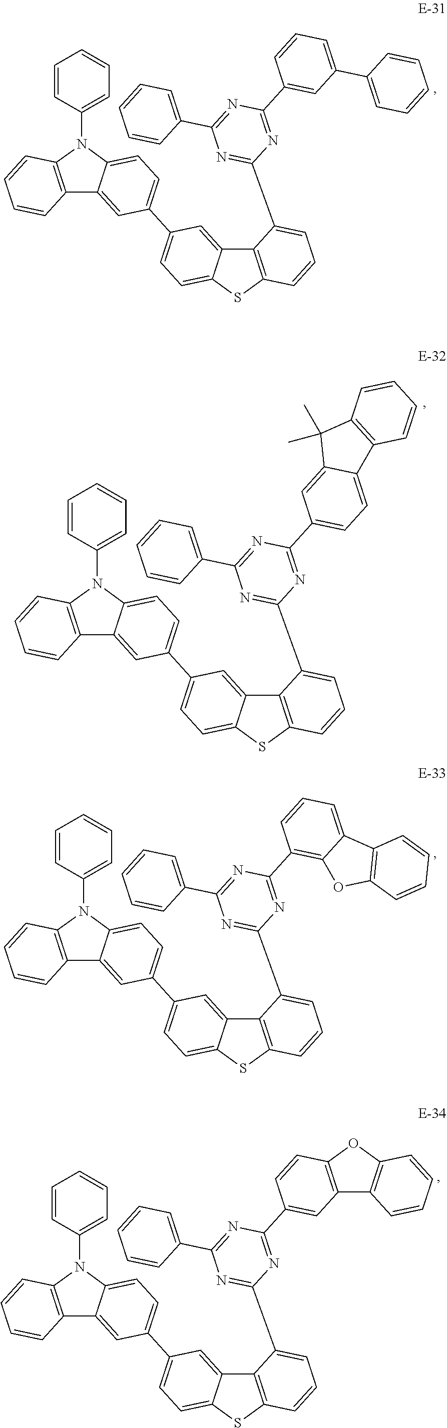 [SMILES: c1ccc(-c2cccc(-c3nc(-c4ccccc4)nc(-c4cccc5sc6ccc(-c7ccc8c(c7)c7ccccc7n8-c7ccccc7)cc6c45)n3)c2)cc1 ; CC1(C)c2ccccc2-c2ccc(-c3nc(-c4ccccc4)nc(-c4cccc5sc6ccc(-c7ccc8c(c7)c7ccccc7n8-c7ccccc7)cc6c45)n3)cc21 ; c1ccc(-c2nc(-c3cccc4c3oc3ccccc34)nc(-c3cccc4sc5ccc(-c6ccc7c(c6)c6ccccc6n7-c6ccccc6)cc5c34)n2)cc1 ; c1ccc(-c2nc(-c3ccc4oc5ccccc5c4c3)nc(-c3cccc4sc5ccc(-c6ccc7c(c6)c6ccccc6n7-c6ccccc6)cc5c34)n2)cc1]

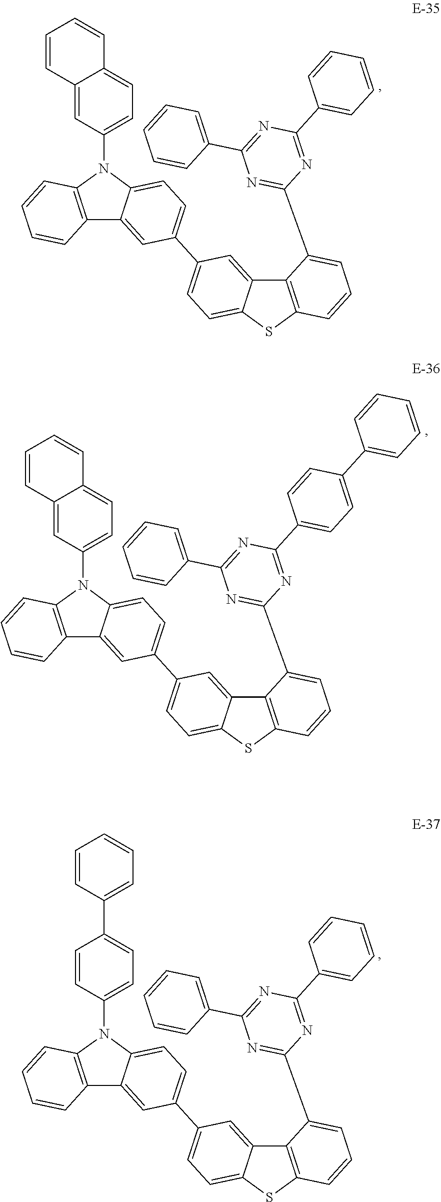 [SMILES: c1ccc(-c2nc(-c3ccccc3)nc(-c3cccc4sc5ccc(-c6ccc7c(c6)c6ccccc6n7-c6ccc7ccccc7c6)cc5c34)n2)cc1 ; c1ccc(-c2ccc(-c3nc(-c4ccccc4)nc(-c4cccc5sc6ccc(-c7ccc8c(c7)c7ccccc7n8-c7ccc8ccccc8c7)cc6c45)n3)cc2)cc1 ; c1ccc(-c2ccc(-n3c4ccccc4c4cc(-c5ccc6sc7cccc(-c8nc(-c9ccccc9)nc(-c9ccccc9)n8)c7c6c5)ccc43)cc2)cc1]

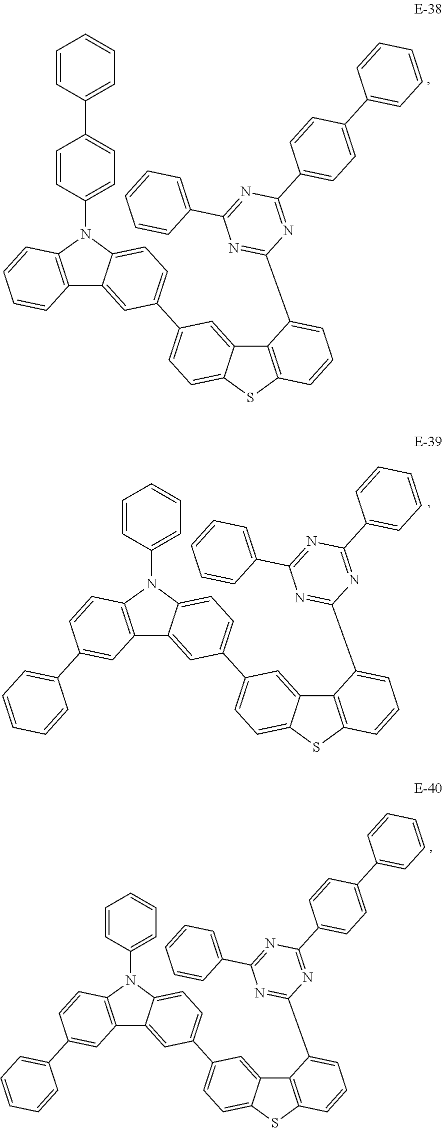 [SMILES: c1ccc(-c2ccc(-c3nc(-c4ccccc4)nc(-c4cccc5sc6ccc(-c7ccc8c(c7)c7ccccc7n8-c7ccc(-c8ccccc8)cc7)cc6c45)n3)cc2)cc1 ; c1ccc(-c2ccc3c(c2)c2cc(-c4ccc5sc6cccc(-c7nc(-c8ccccc8)nc(-c8ccccc8)n7)c6c5c4)ccc2n3-c2ccccc2)cc1 ; c1ccc(-c2ccc(-c3nc(-c4ccccc4)nc(-c4cccc5sc6ccc(-c7ccc8c(c7)c7cc(-c9ccccc9)ccc7n8-c7ccccc7)cc6c45)n3)cc2)cc1]

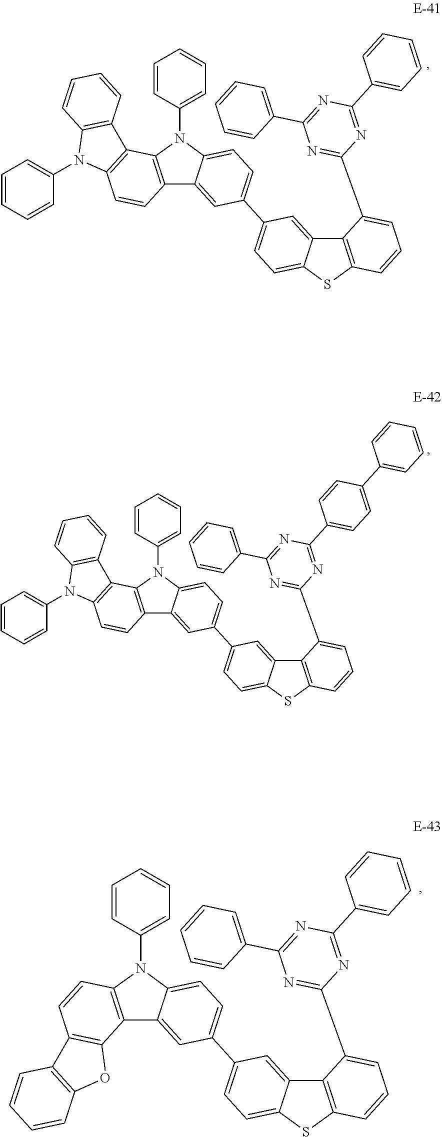 [SMILES: c1ccc(-c2nc(-c3ccccc3)nc(-c3cccc4sc5ccc(-c6ccc7c(c6)c6ccc8c(c9ccccc9n8-c8ccccc8)c6n7-c6ccccc6)cc5c34)n2)cc1 ; c1ccc(-c2ccc(-c3nc(-c4ccccc4)nc(-c4cccc5sc6ccc(-c7ccc8c(c7)c7ccc9c(c%10ccccc%10n9-c9ccccc9)c7n8-c7ccccc7)cc6c45)n3)cc2)cc1 ; c1ccc(-c2nc(-c3ccccc3)nc(-c3cccc4sc5ccc(-c6ccc7c(c6)c6c8oc9ccccc9c8ccc6n7-c6ccccc6)cc5c34)n2)cc1]

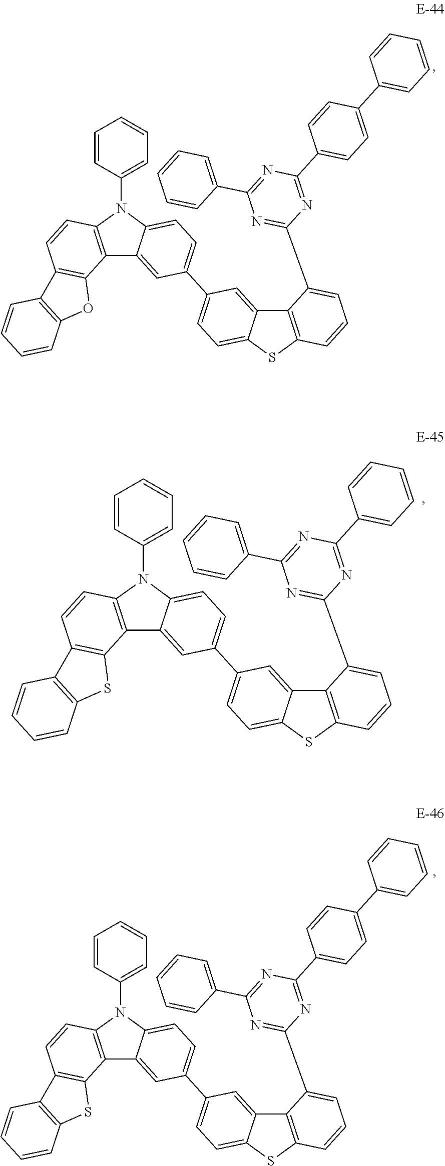 [SMILES: c1ccc(-c2ccc(-c3nc(-c4ccccc4)nc(-c4cccc5sc6ccc(-c7ccc8c(c7)c7c9oc%10ccccc%10c9ccc7n8-c7ccccc7)cc6c45)n3)cc2)cc1 ; c1ccc(-c2nc(-c3ccccc3)nc(-c3cccc4sc5ccc(-c6ccc7c(c6)c6c8sc9ccccc9c8ccc6n7-c6ccccc6)cc5c34)n2)cc1 ; c1ccc(-c2ccc(-c3nc(-c4ccccc4)nc(-c4cccc5sc6ccc(-c7ccc8c(c7)c7c9sc%10ccccc%10c9ccc7n8-c7ccccc7)cc6c45)n3)cc2)cc1]

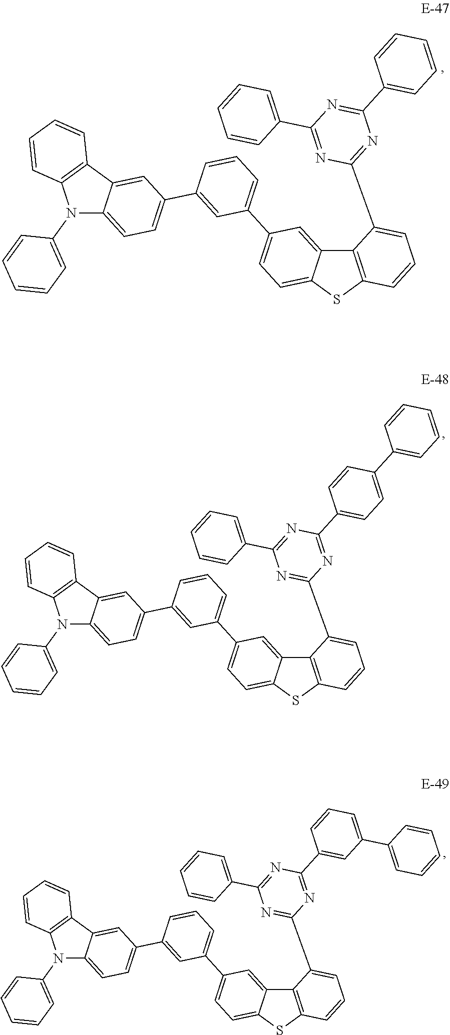 [SMILES: c1ccc(-c2nc(-c3ccccc3)nc(-c3cccc4sc5ccc(-c6cccc(-c7ccc8c(c7)c7ccccc7n8-c7ccccc7)c6)cc5c34)n2)cc1 ; c1ccc(-c2ccc(-c3nc(-c4ccccc4)nc(-c4cccc5sc6ccc(-c7cccc(-c8ccc9c(c8)c8ccccc8n9-c8ccccc8)c7)cc6c45)n3)cc2)cc1 ; c1ccc(-c2cccc(-c3nc(-c4ccccc4)nc(-c4cccc5sc6ccc(-c7cccc(-c8ccc9c(c8)c8ccccc8n9-c8ccccc8)c7)cc6c45)n3)c2)cc1]

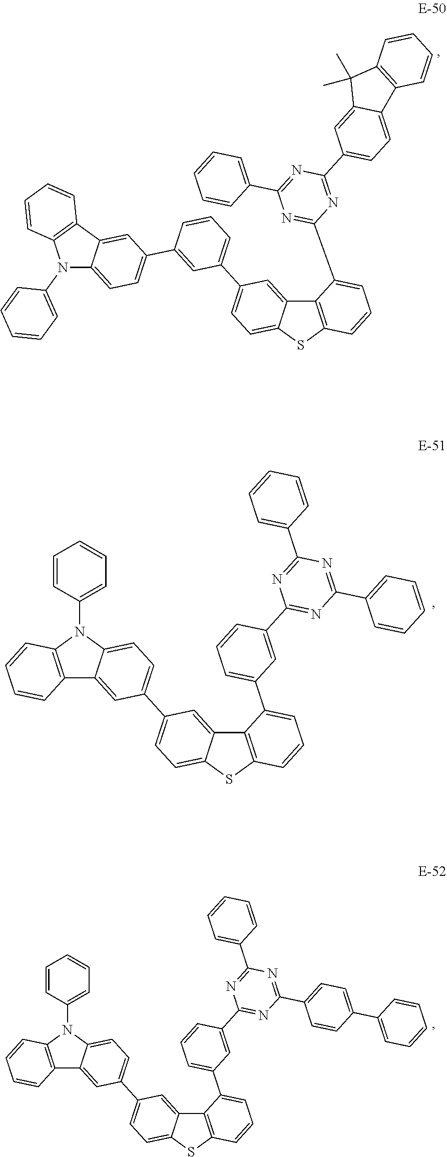 [SMILES: CC1(C)c2ccccc2-c2ccc(-c3nc(-c4ccccc4)nc(-c4cccc5sc6ccc(-c7cccc(-c8ccc9c(c8)c8ccccc8n9-c8ccccc8)c7)cc6c45)n3)cc21 ; c1ccc(-c2nc(-c3ccccc3)nc(-c3cccc(-c4cccc5sc6ccc(-c7ccc8c(c7)c7ccccc7n8-c7ccccc7)cc6c45)c3)n2)cc1 ; c1ccc(-c2ccc(-c3nc(-c4ccccc4)nc(-c4cccc(-c5cccc6sc7ccc(-c8ccc9c(c8)c8ccccc8n9-c8ccccc8)cc7c56)c4)n3)cc2)cc1]

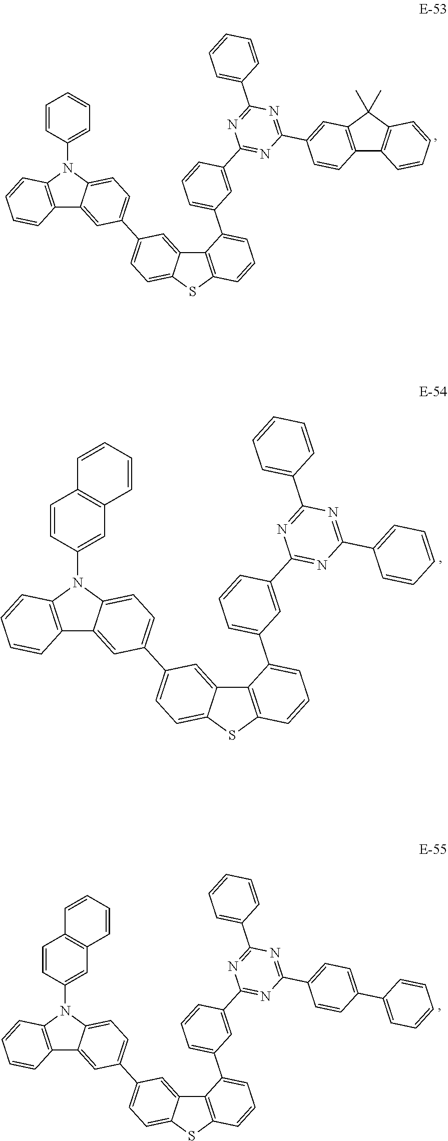 [SMILES: CC1(C)c2ccccc2-c2ccc(-c3nc(-c4ccccc4)nc(-c4cccc(-c5cccc6sc7ccc(-c8ccc9c(c8)c8ccccc8n9-c8ccccc8)cc7c56)c4)n3)cc21 ; c1ccc(-c2nc(-c3ccccc3)nc(-c3cccc(-c4cccc5sc6ccc(-c7ccc8c(c7)c7ccccc7n8-c7ccc8ccccc8c7)cc6c45)c3)n2)cc1 ; c1ccc(-c2ccc(-c3nc(-c4ccccc4)nc(-c4cccc(-c5cccc6sc7ccc(-c8ccc9c(c8)c8ccccc8n9-c8ccc9ccccc9c8)cc7c56)c4)n3)cc2)cc1]

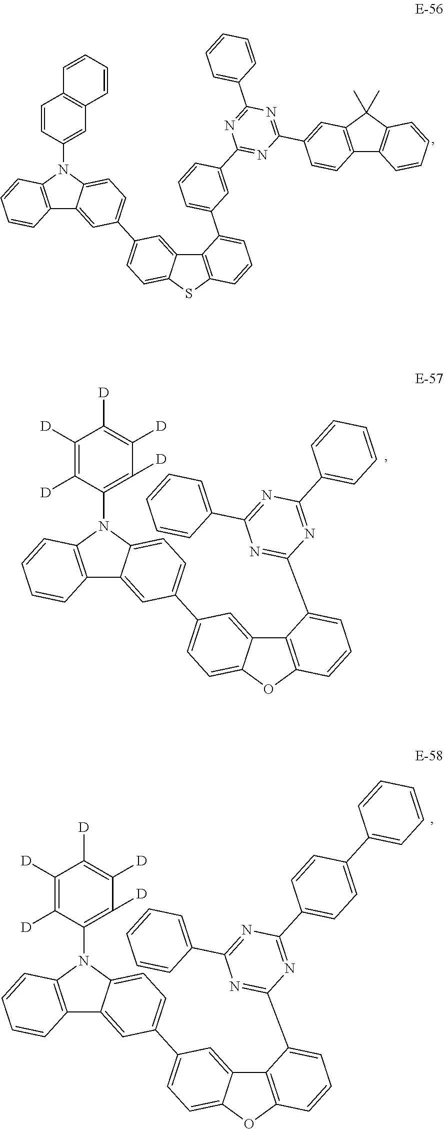 [SMILES: CC1(C)c2ccccc2-c2ccc(-c3nc(-c4ccccc4)nc(-c4cccc(-c5cccc6sc7ccc(-c8ccc9c(c8)c8ccccc8n9-c8ccc9ccccc9c8)cc7c56)c4)n3)cc21 ; [2H]c1c([2H])c([2H])c(-n2c3ccccc3c3cc(-c4ccc5oc6cccc(-c7nc(-c8ccccc8)nc(-c8ccccc8)n7)c6c5c4)ccc32)c([2H])c1[2H] ; [2H]c1c([2H])c([2H])c(-n2c3ccccc3c3cc(-c4ccc5oc6cccc(-c7nc(-c8ccccc8)nc(-c8ccc(-c9ccccc9)cc8)n7)c6c5c4)ccc32)c([2H])c1[2H]]

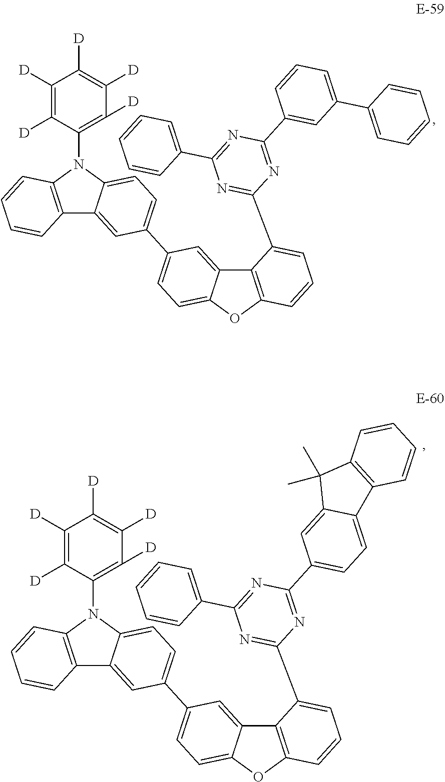 [SMILES: [2H]c1c([2H])c([2H])c(-n2c3ccccc3c3cc(-c4ccc5oc6cccc(-c7nc(-c8ccccc8)nc(-c8cccc(-c9ccccc9)c8)n7)c6c5c4)ccc32)c([2H])c1[2H] ; [2H]c1c([2H])c([2H])c(-n2c3ccccc3c3cc(-c4ccc5oc6cccc(-c7nc(-c8ccccc8)nc(-c8ccc9c(c8)C(C)(C)c8ccccc8-9)n7)c6c5c4)ccc32)c([2H])c1[2H]]

18. The device of claim 1, wherein the dopant compound is selected from the group consisting of:

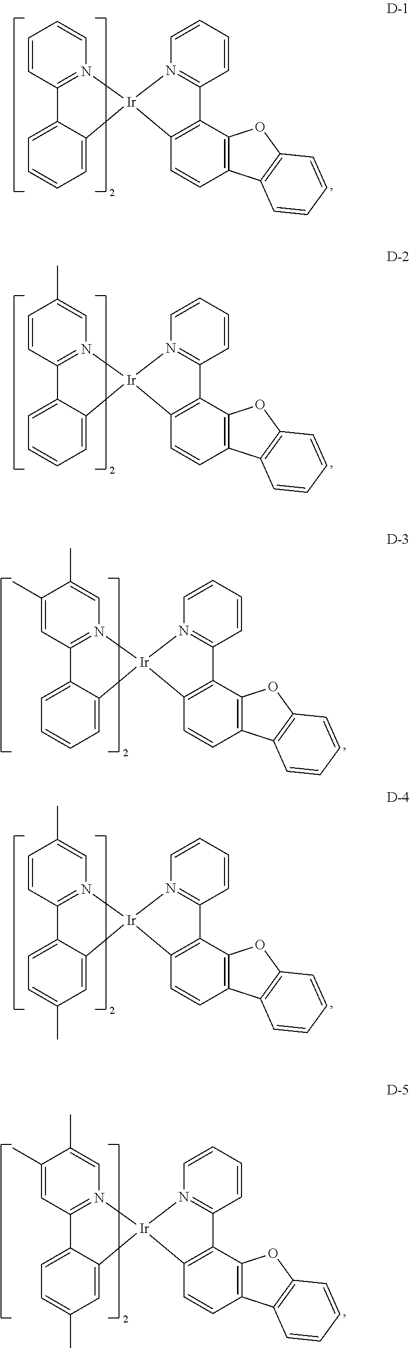

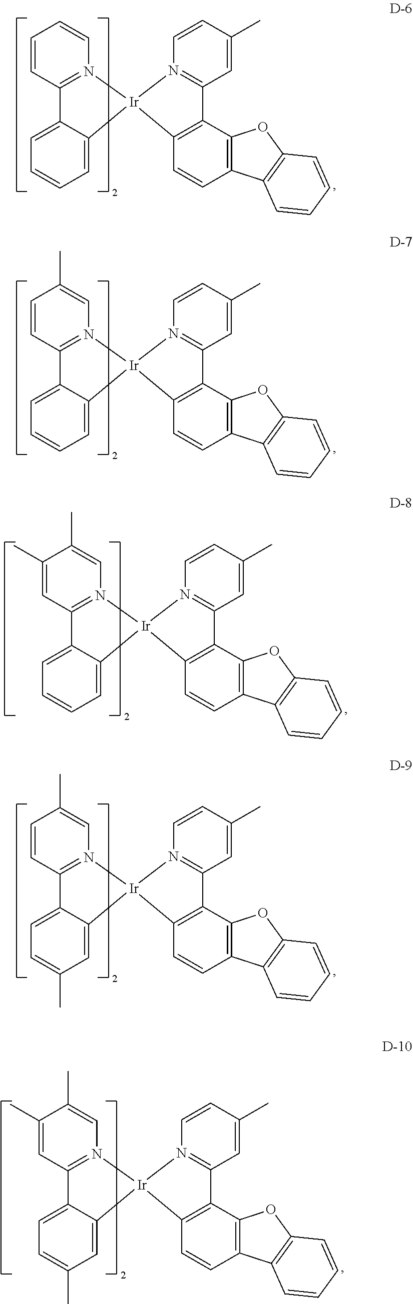

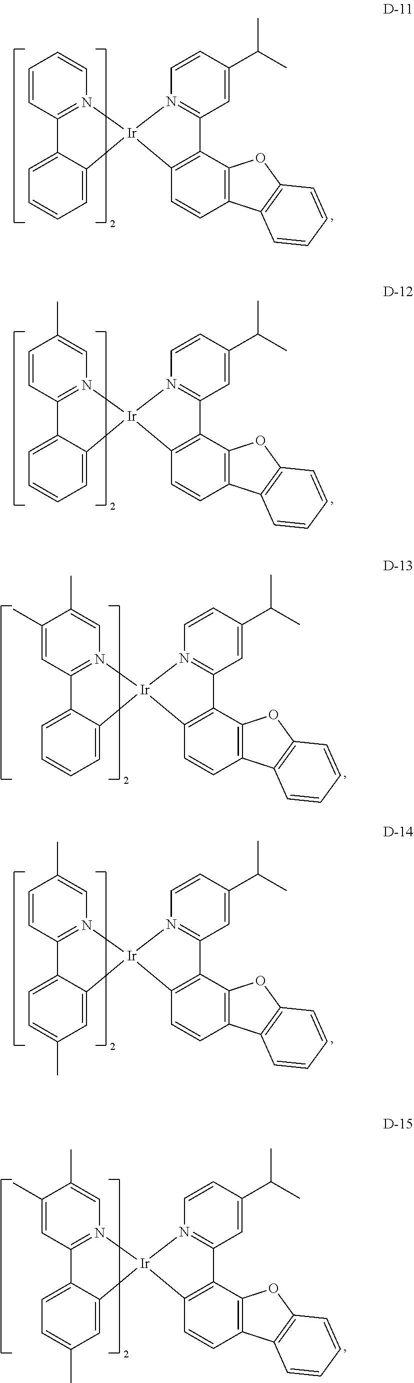

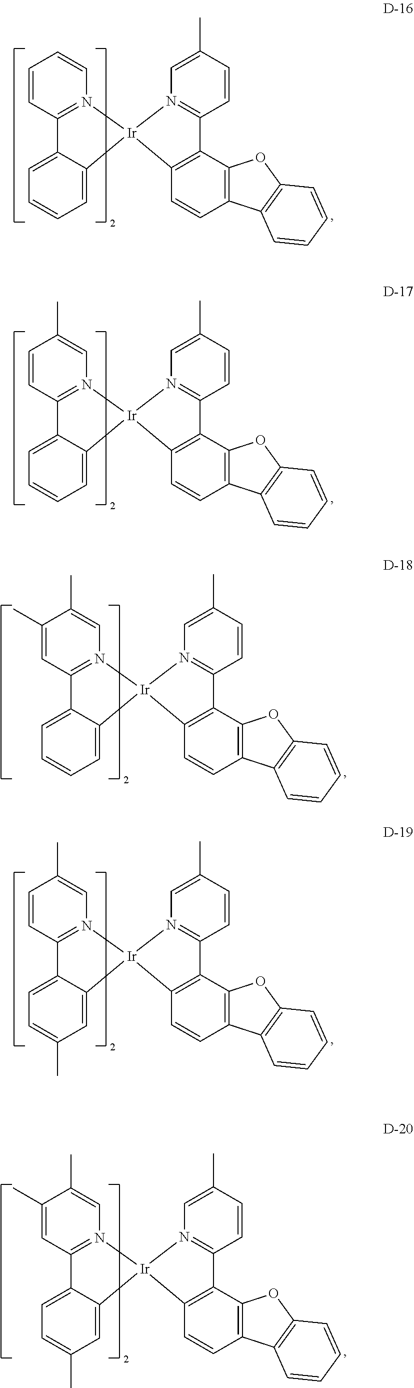

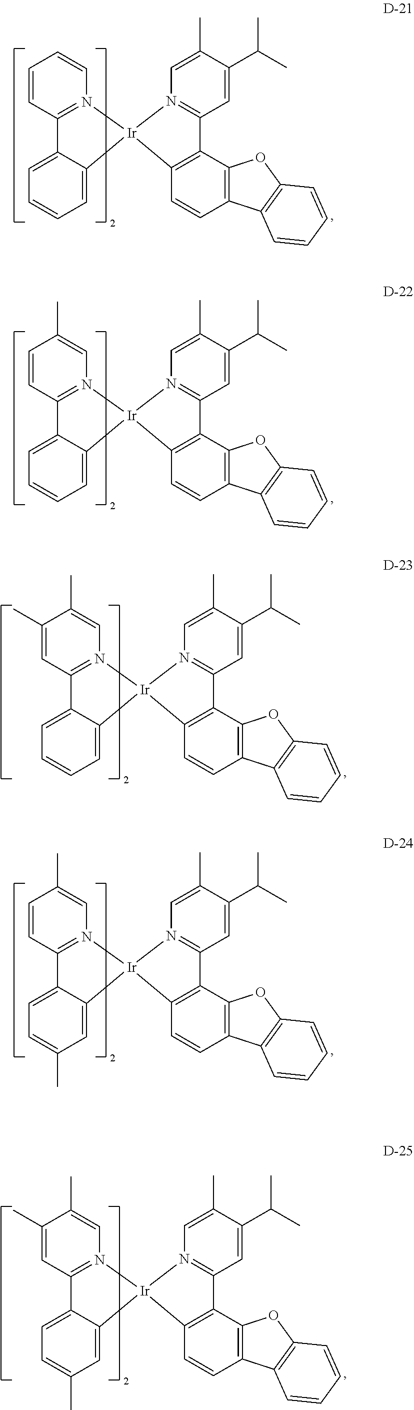

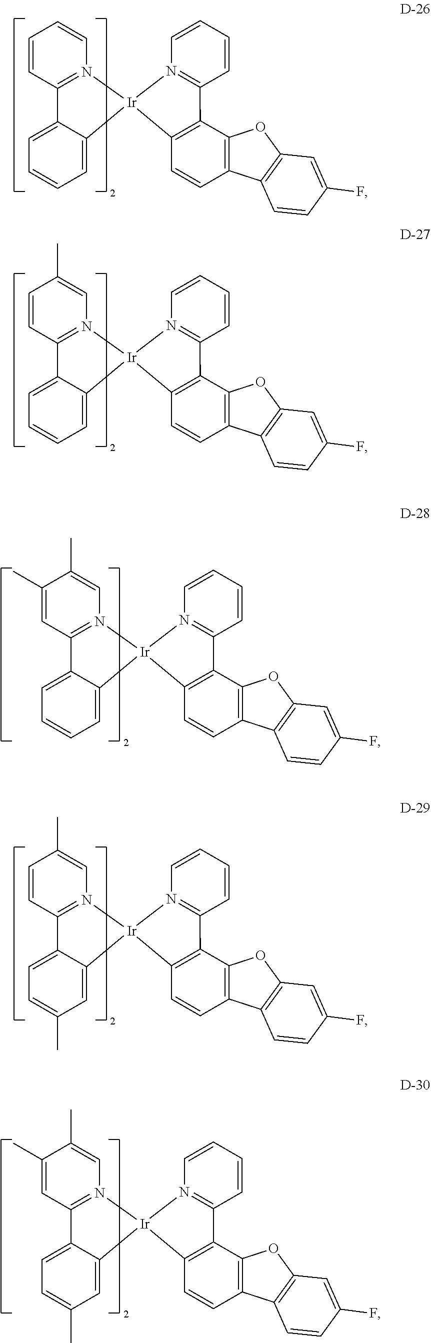

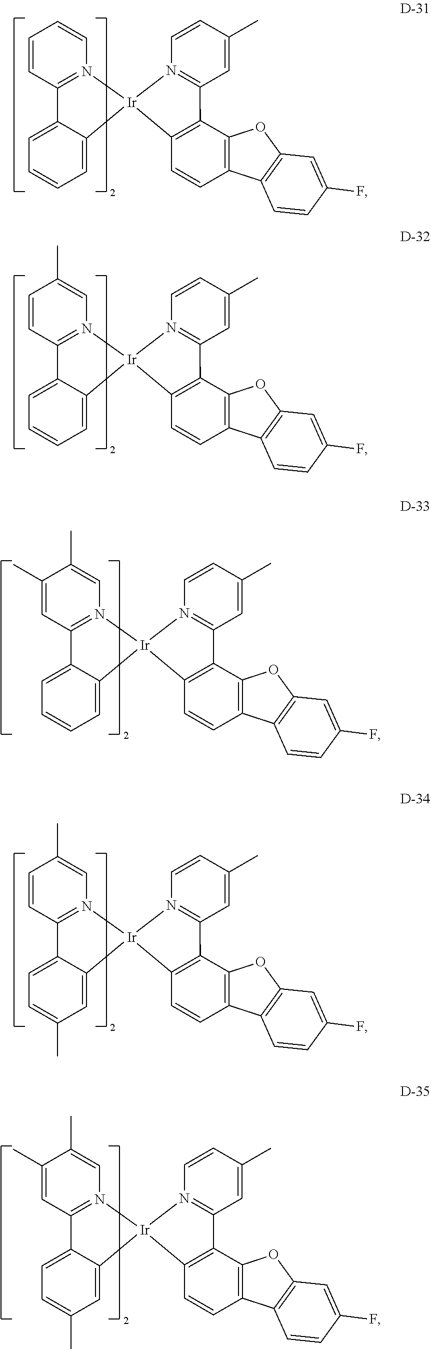

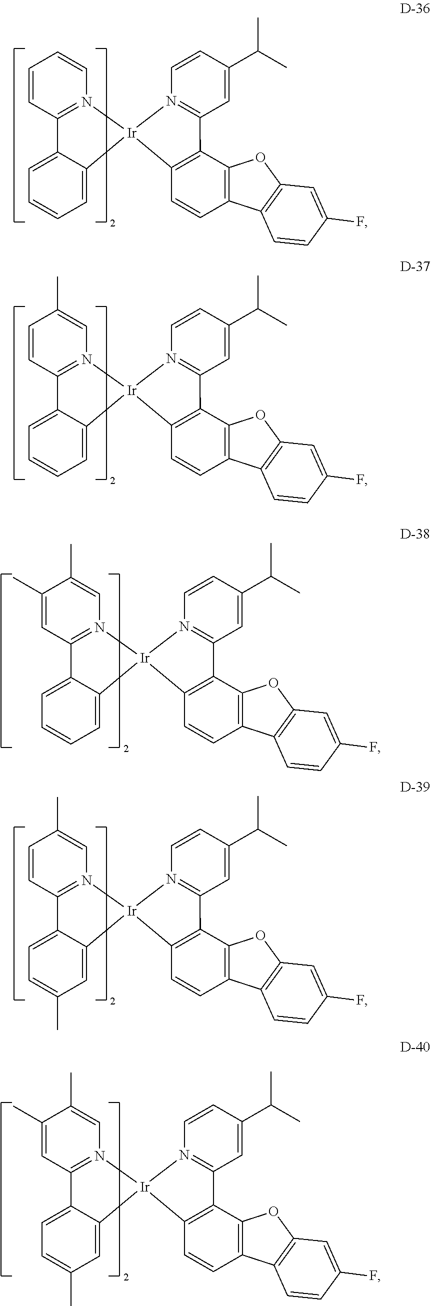

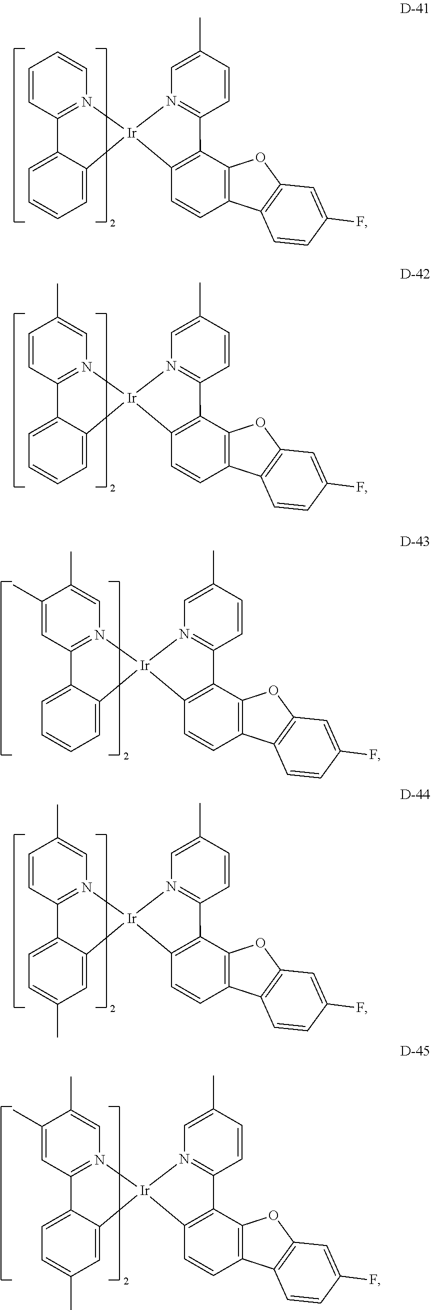

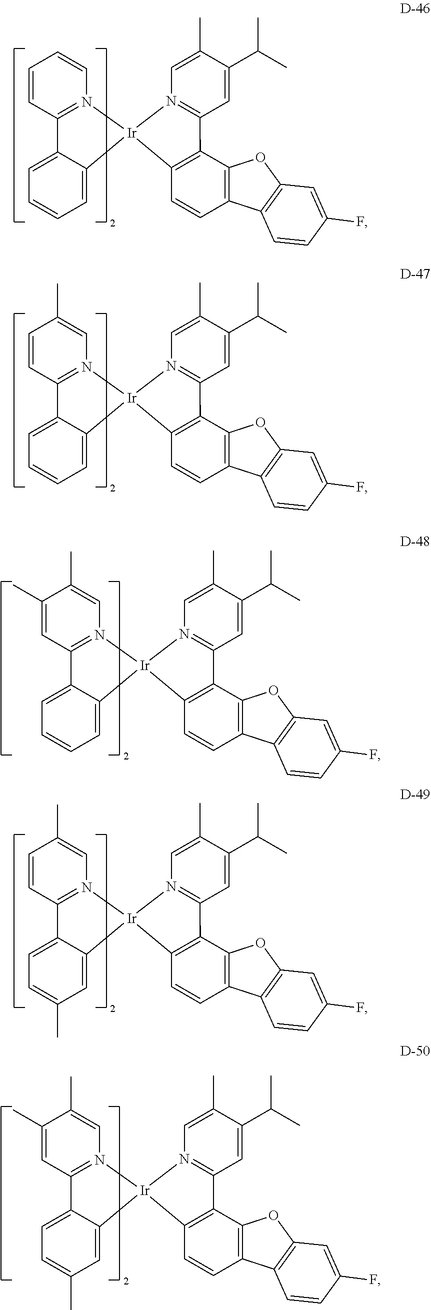

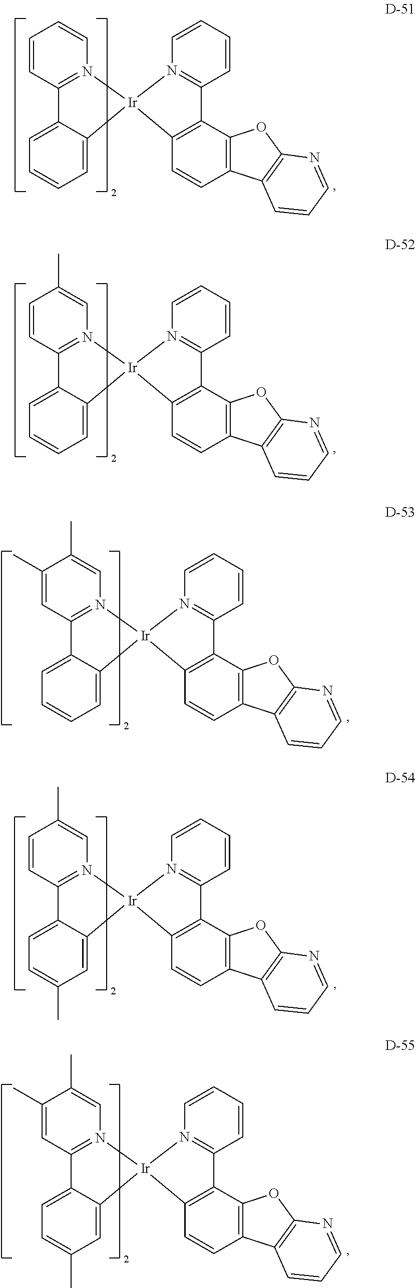

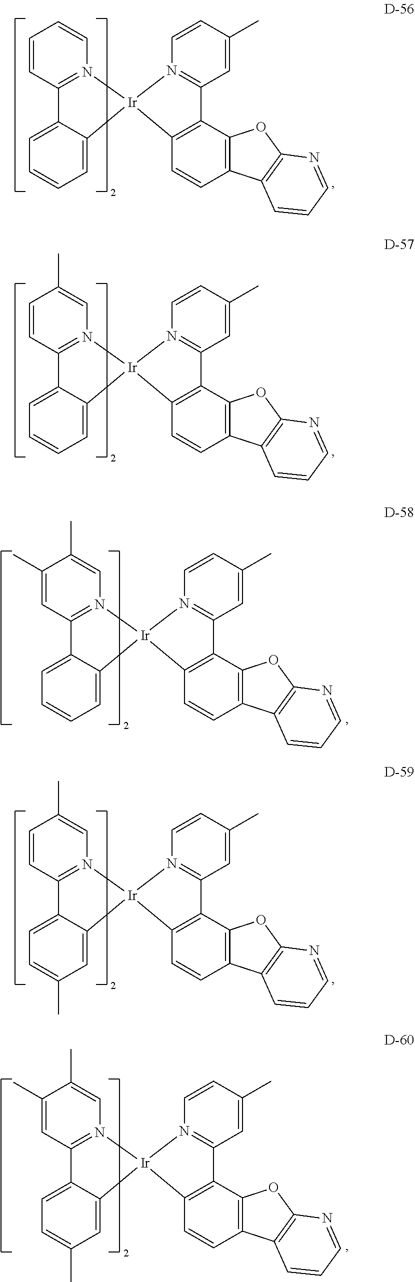

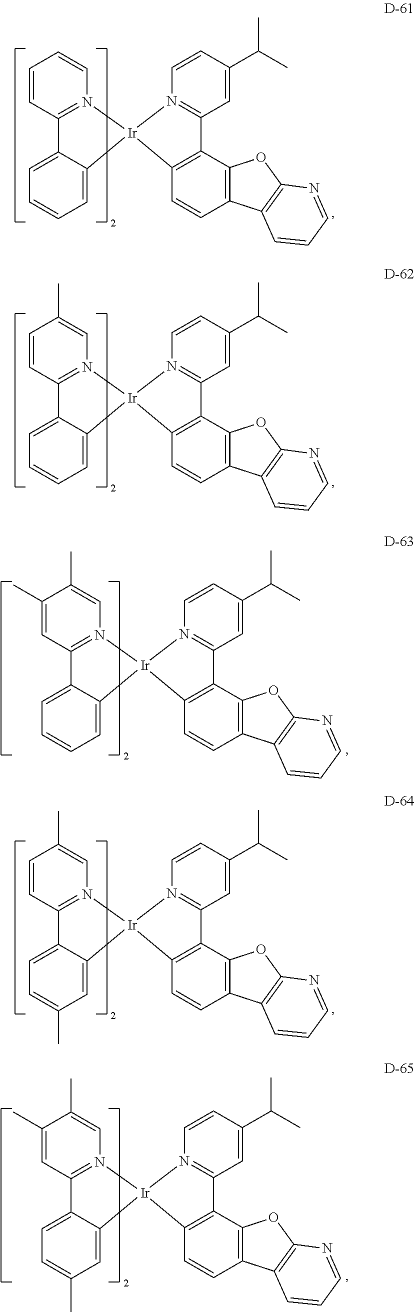

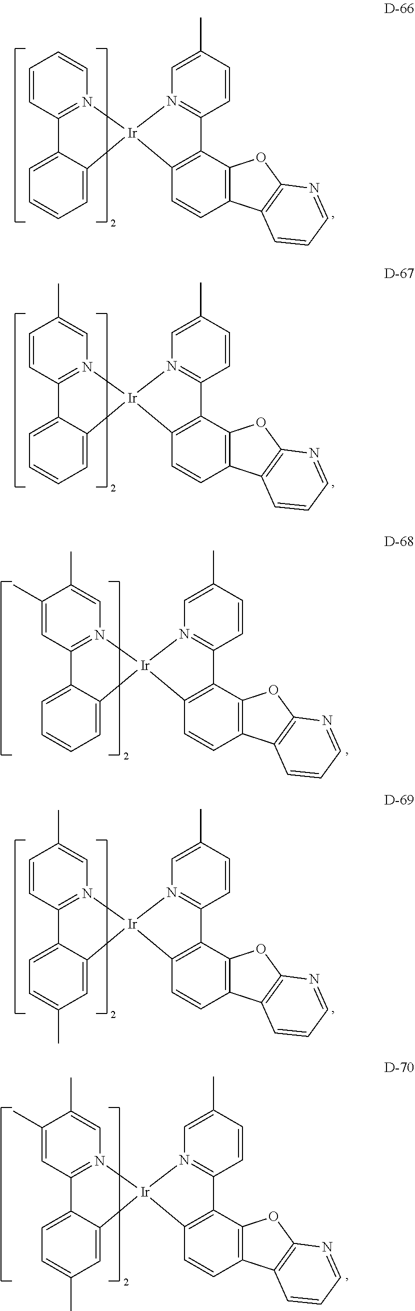

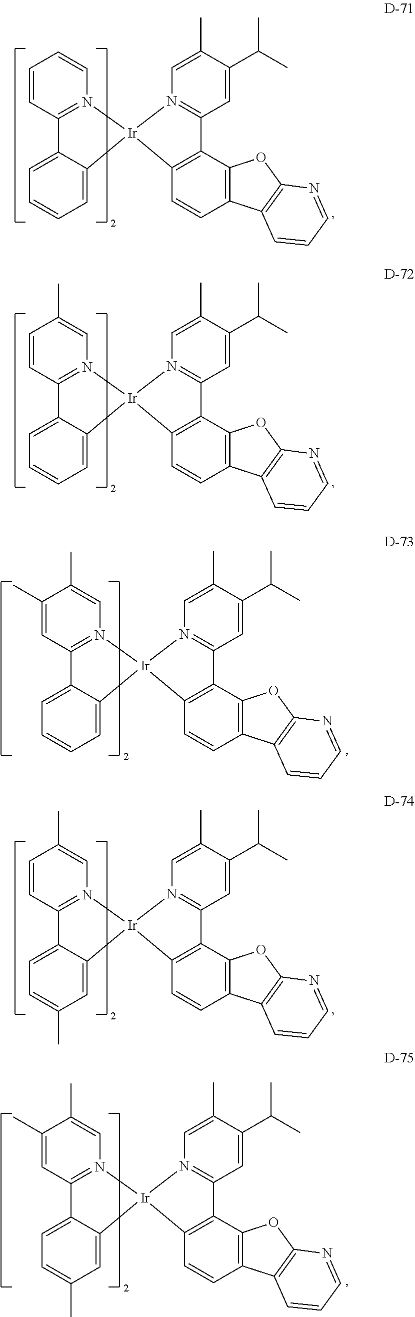

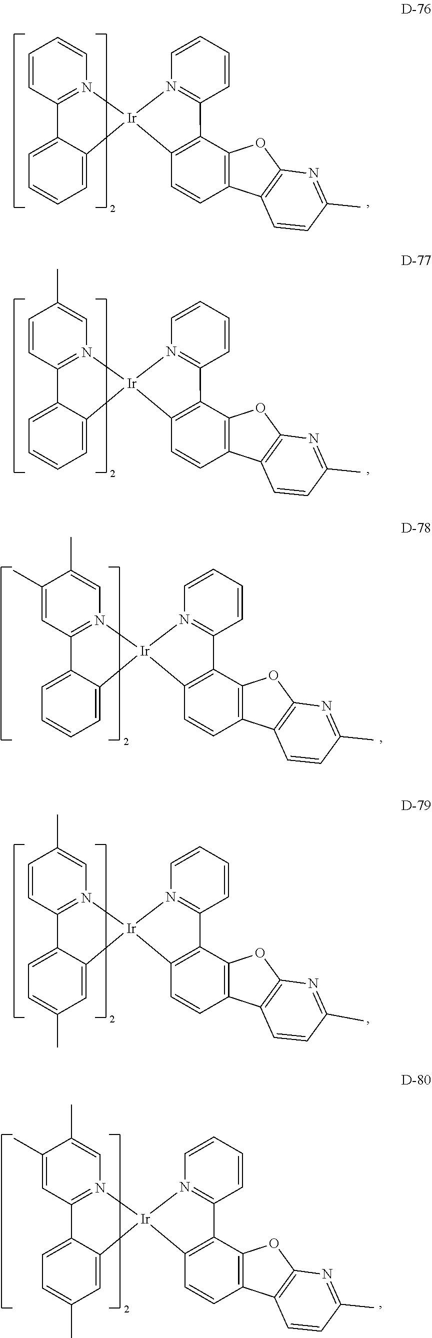

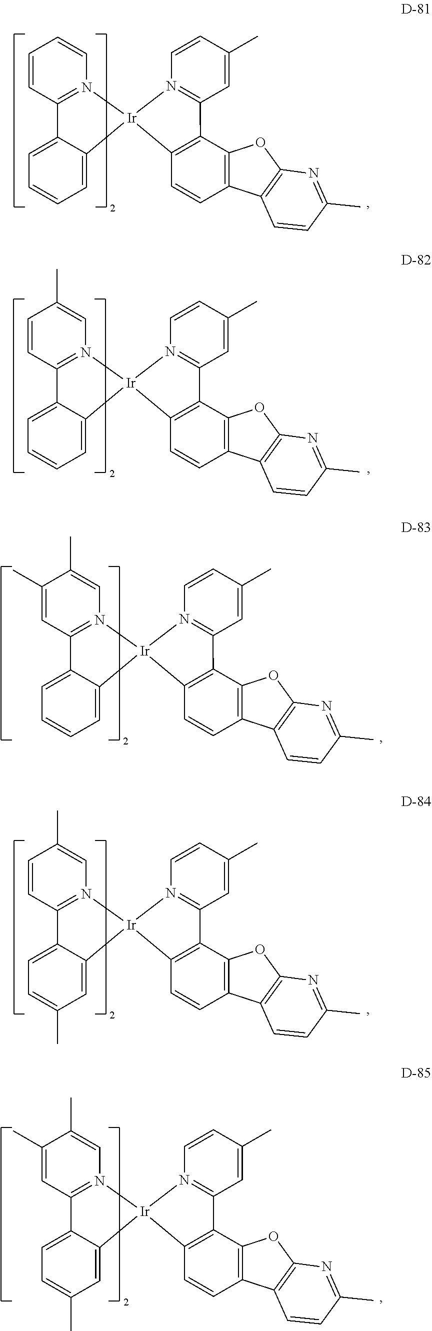

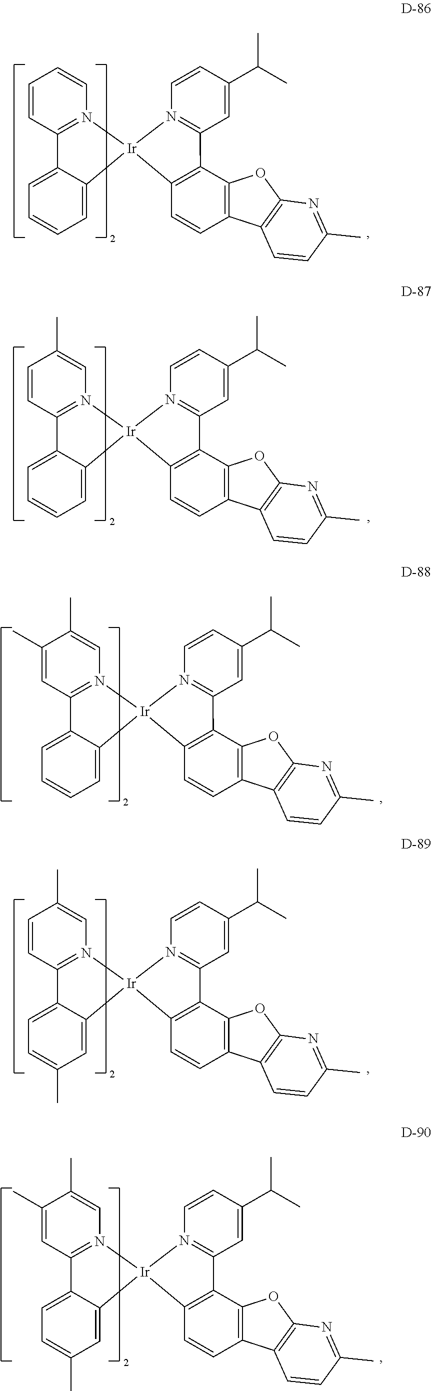

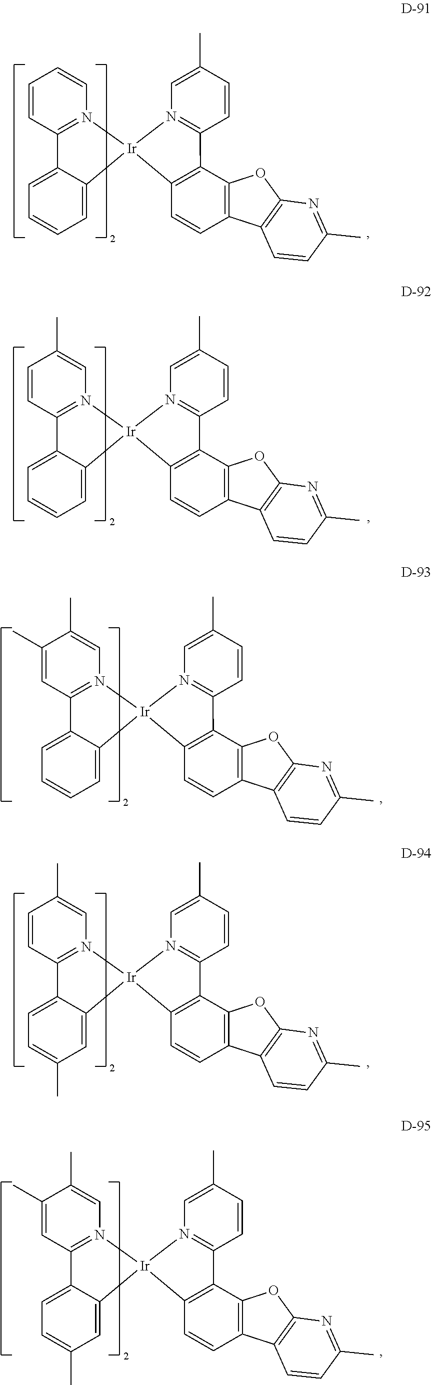

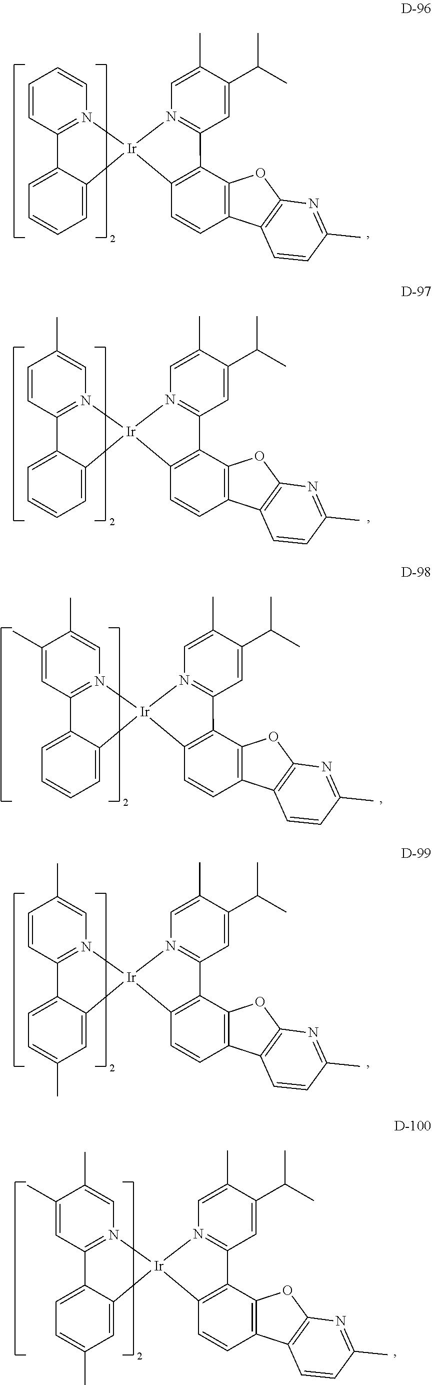

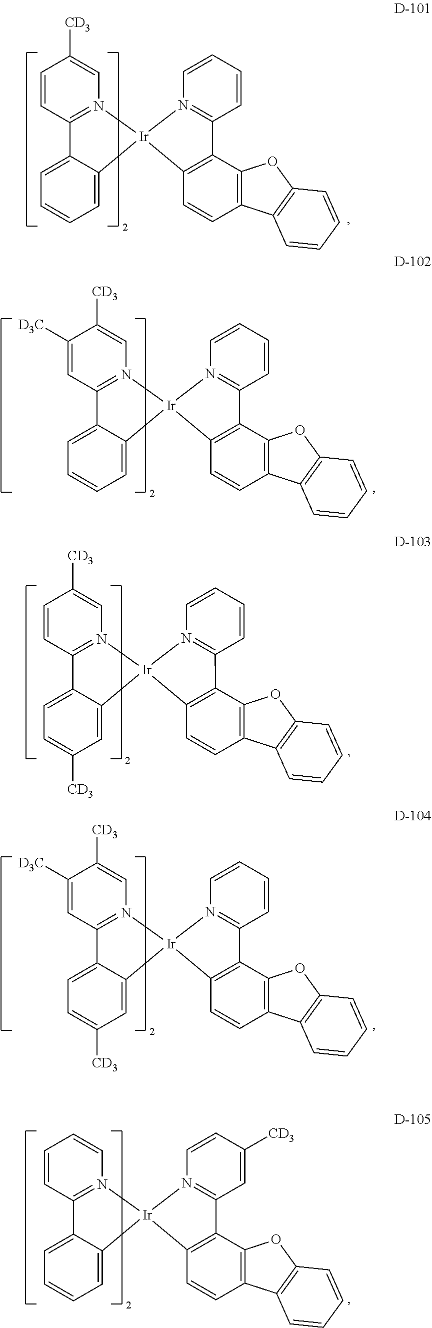

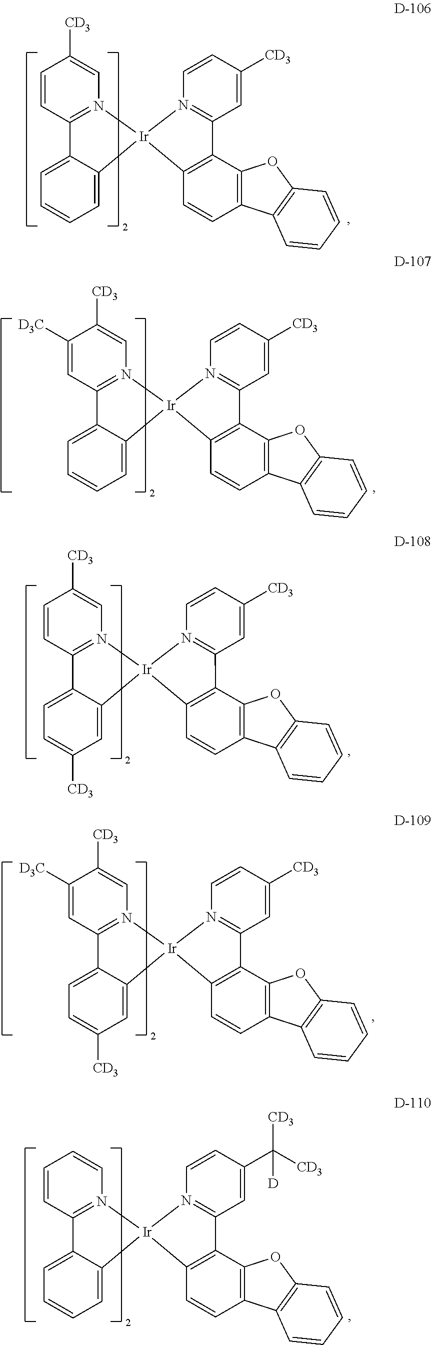

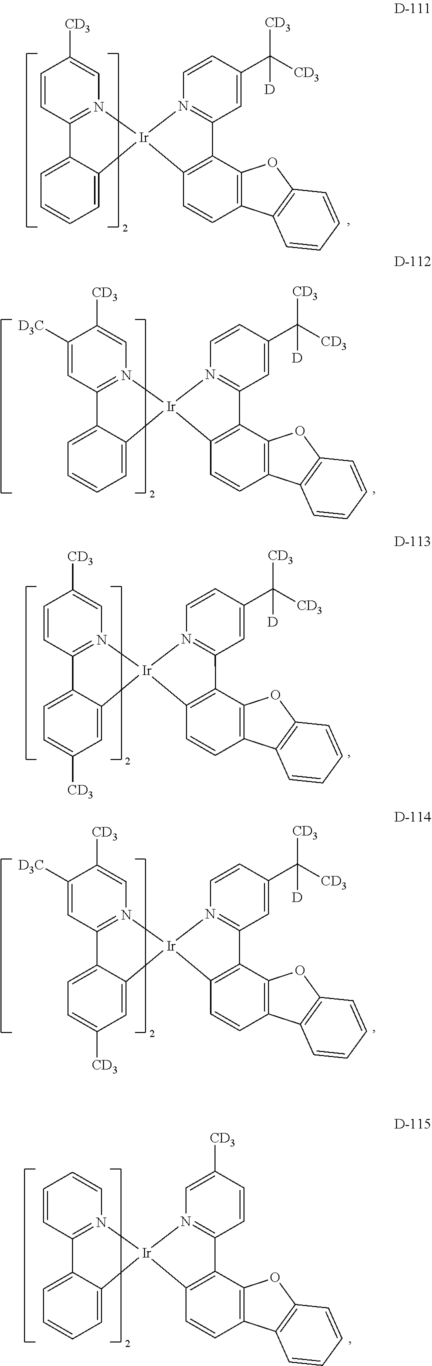

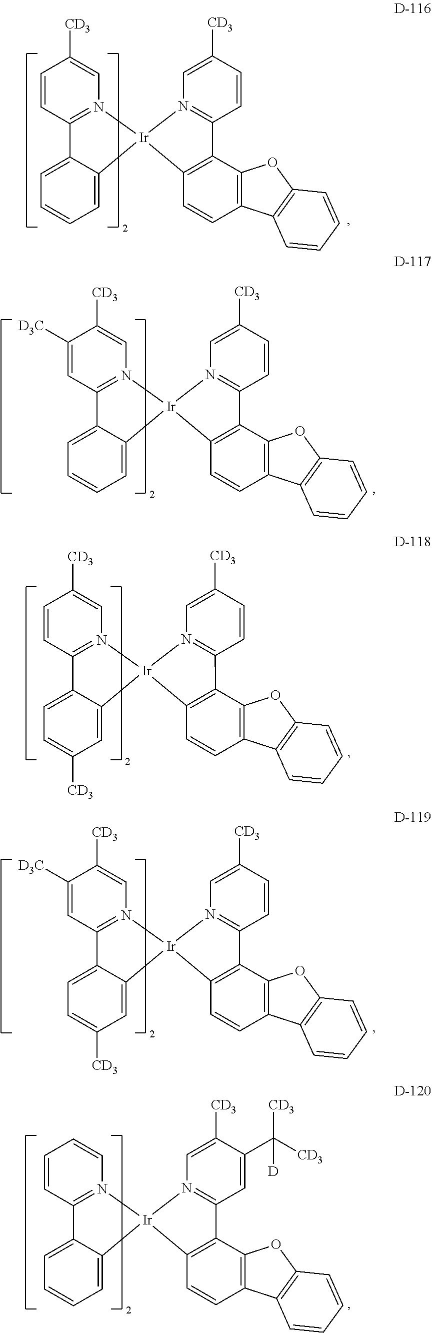

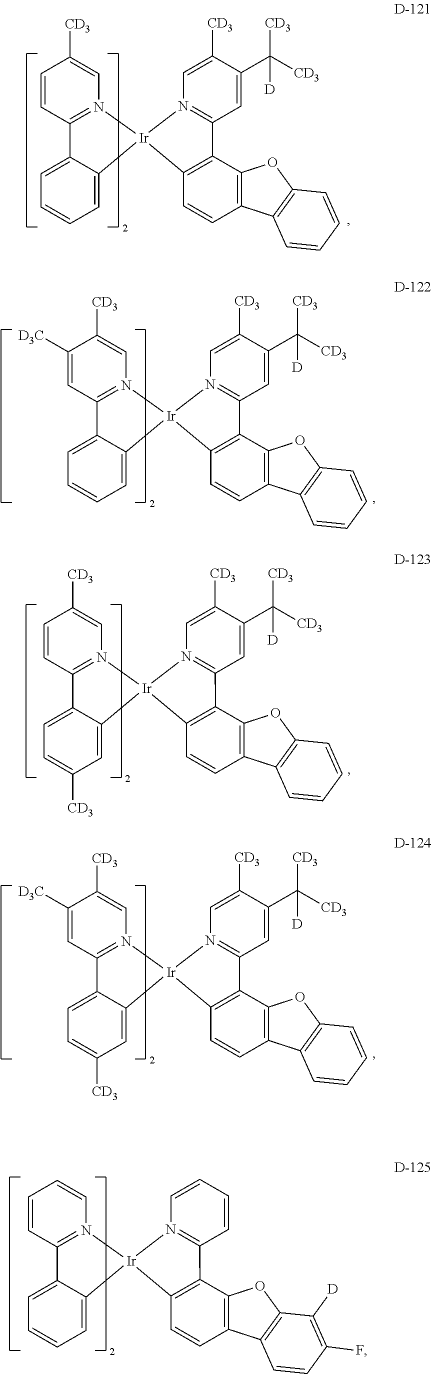

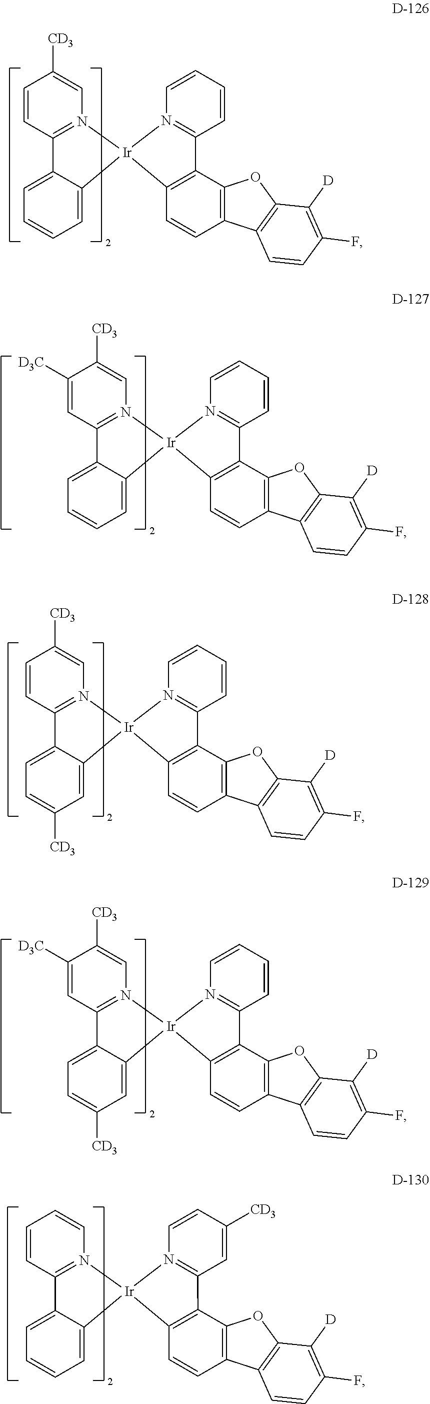

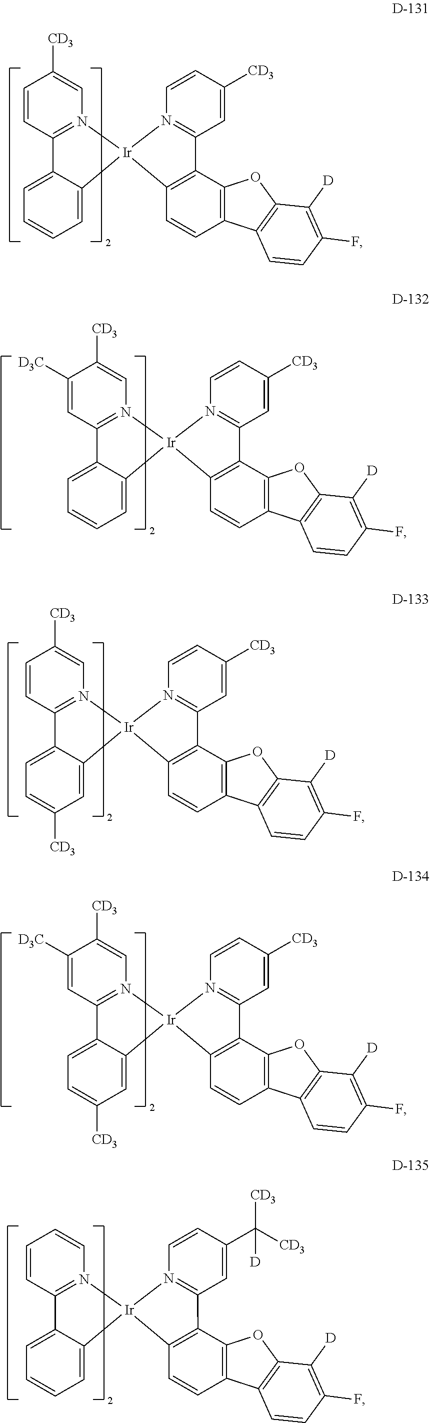

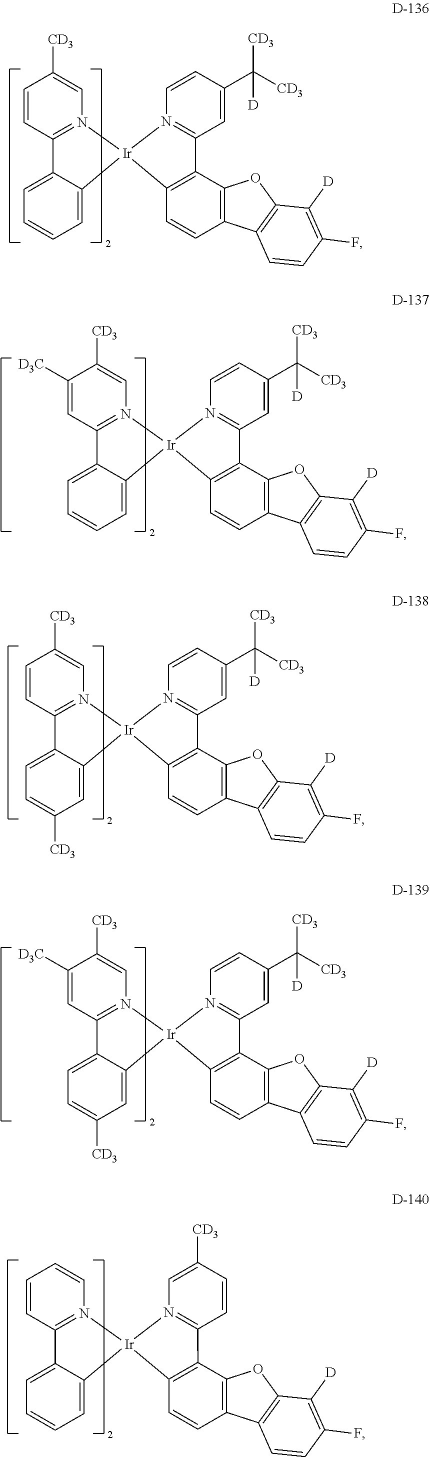

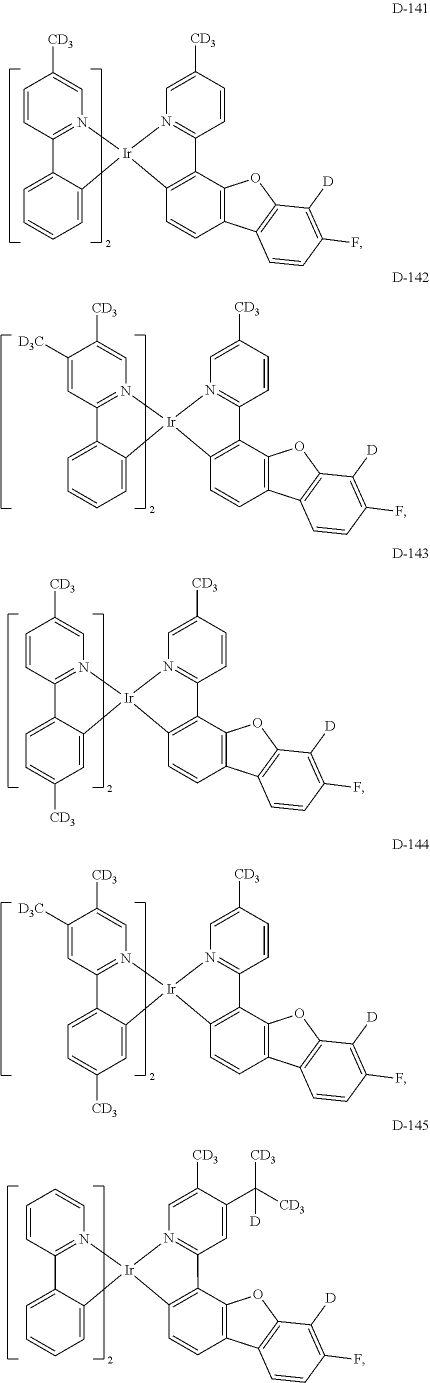

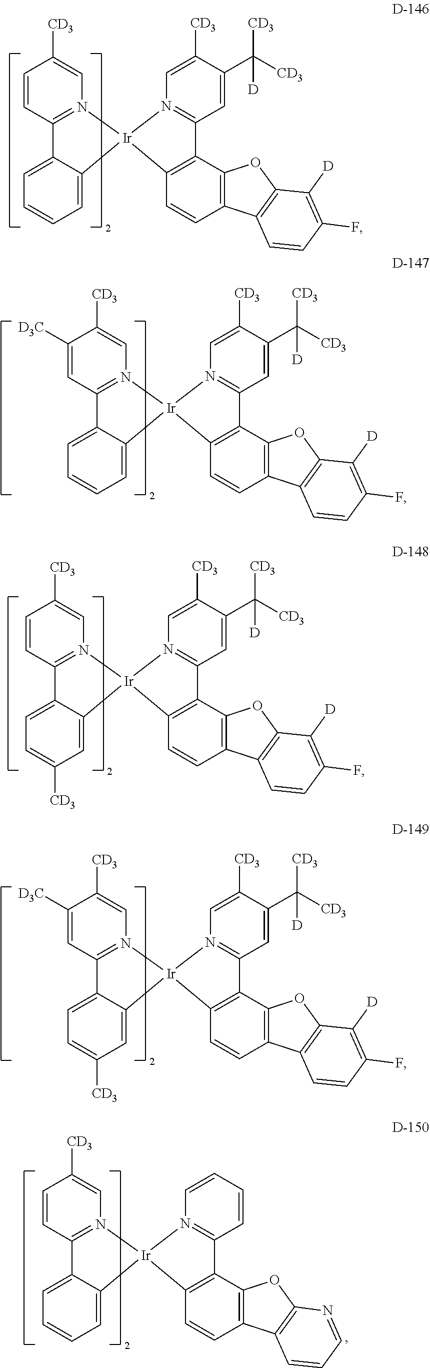

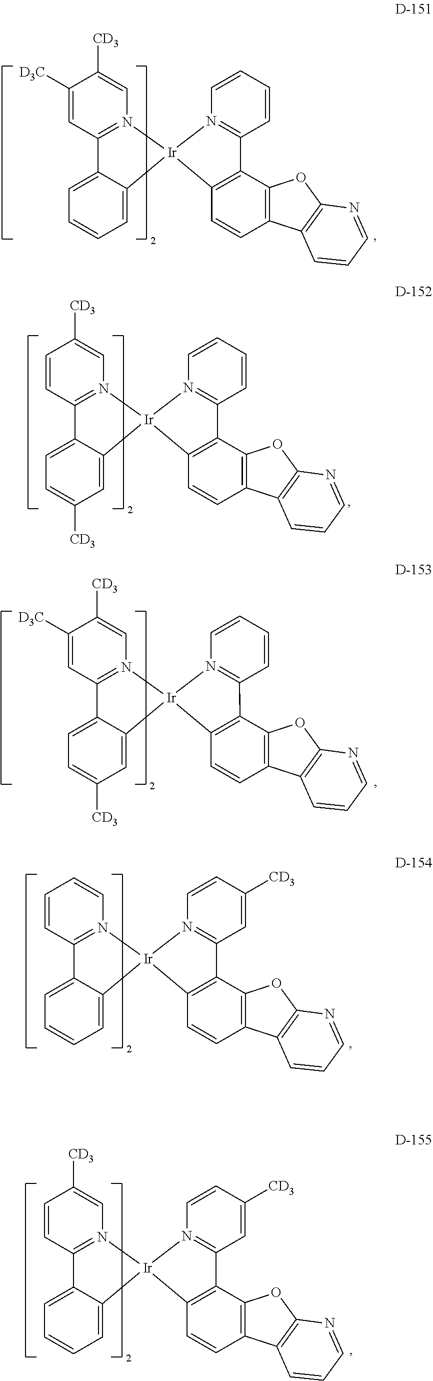

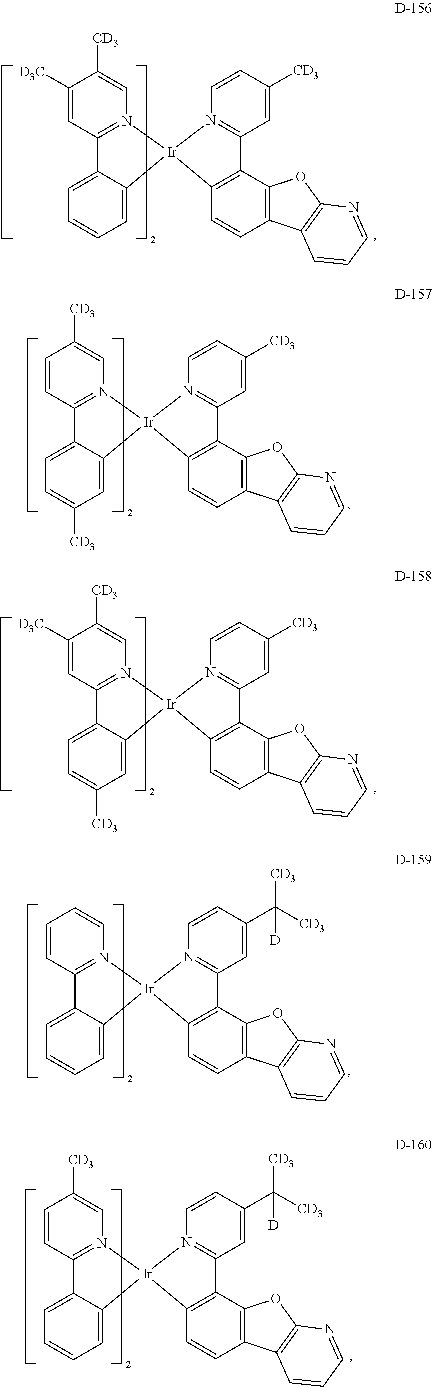

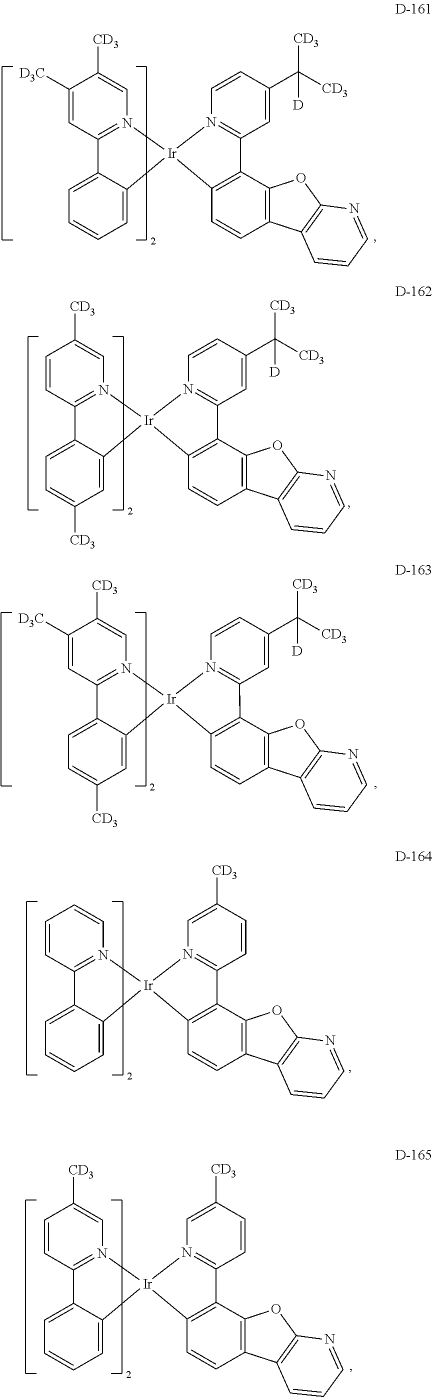

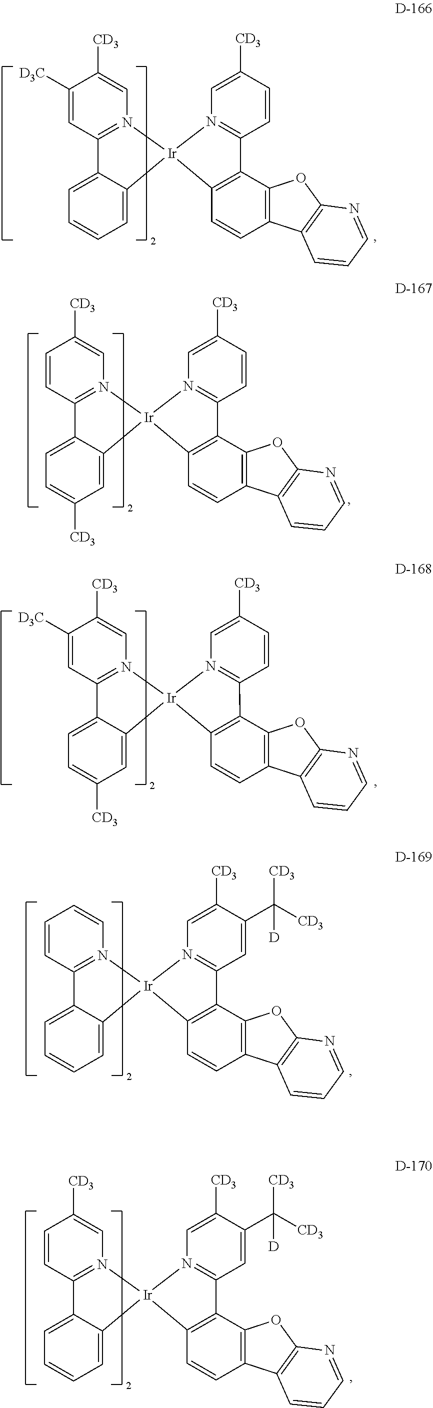

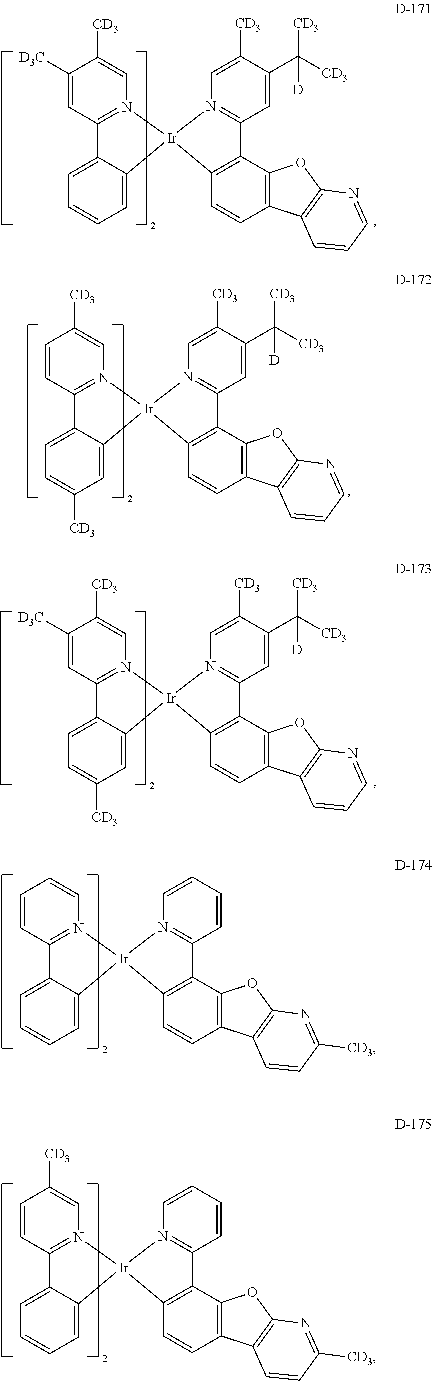

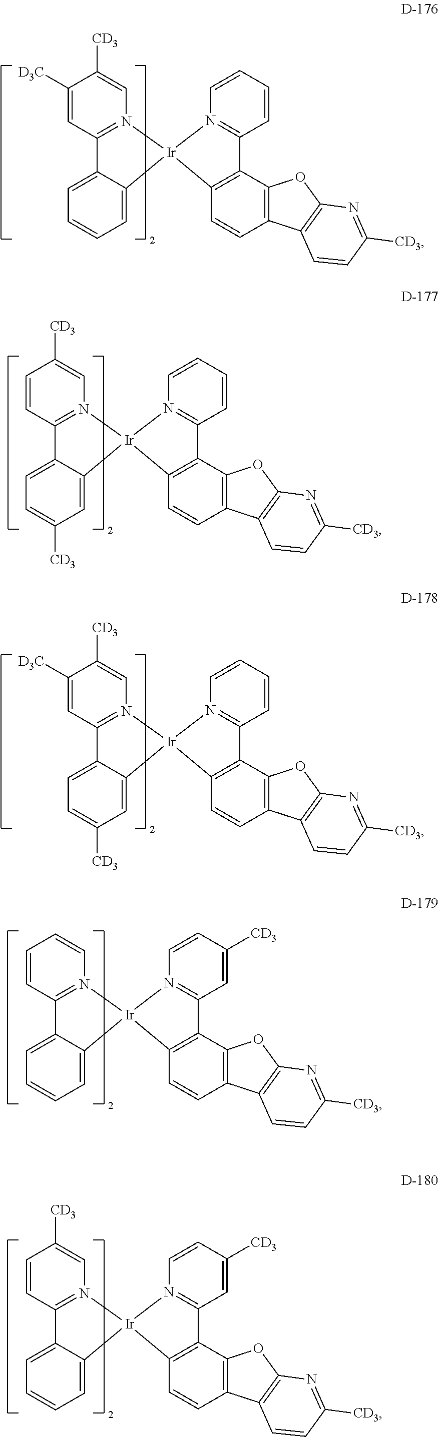

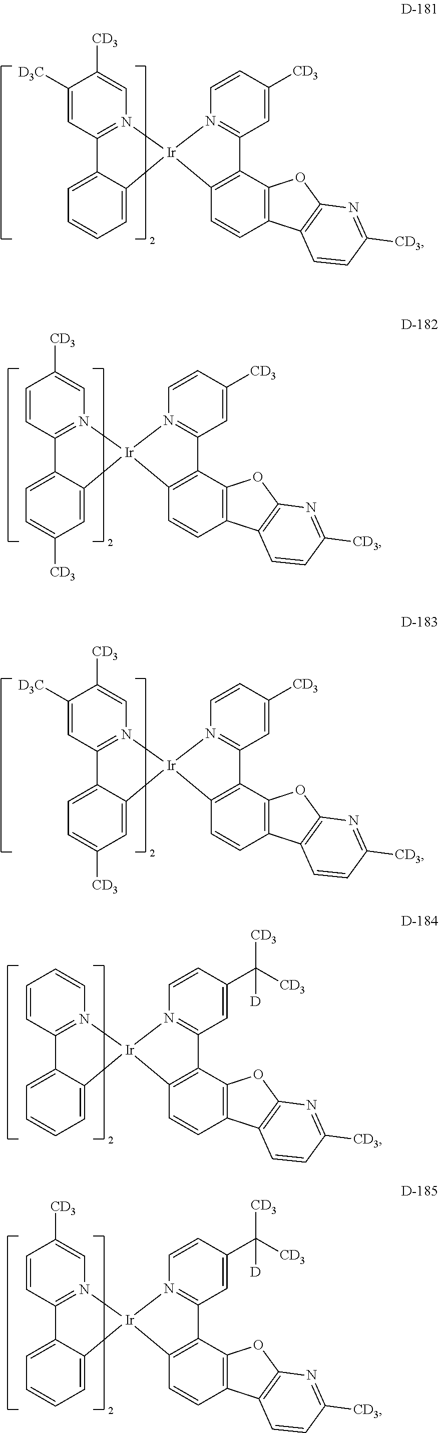

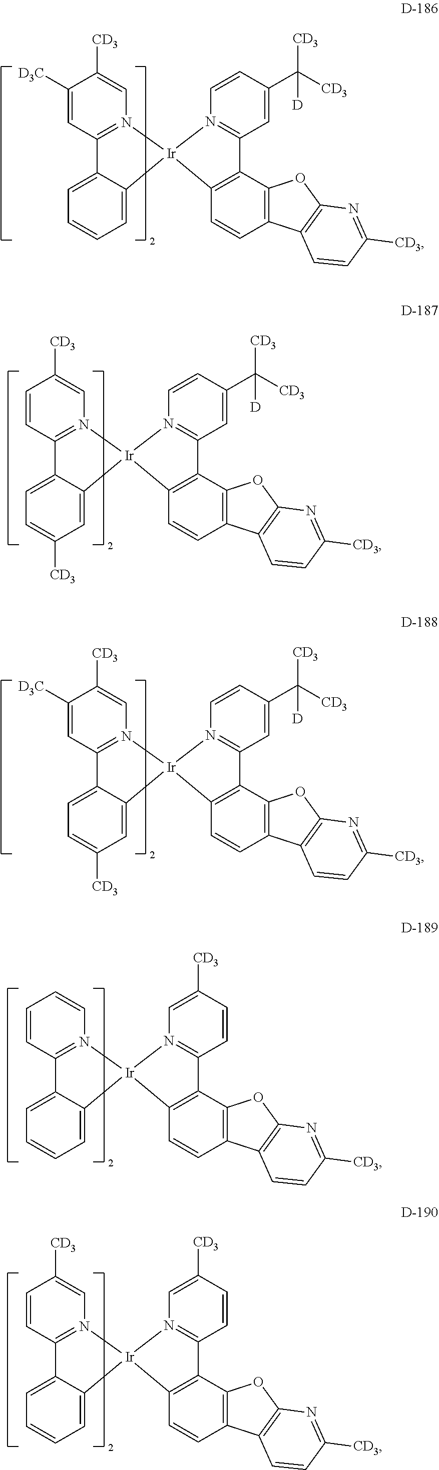

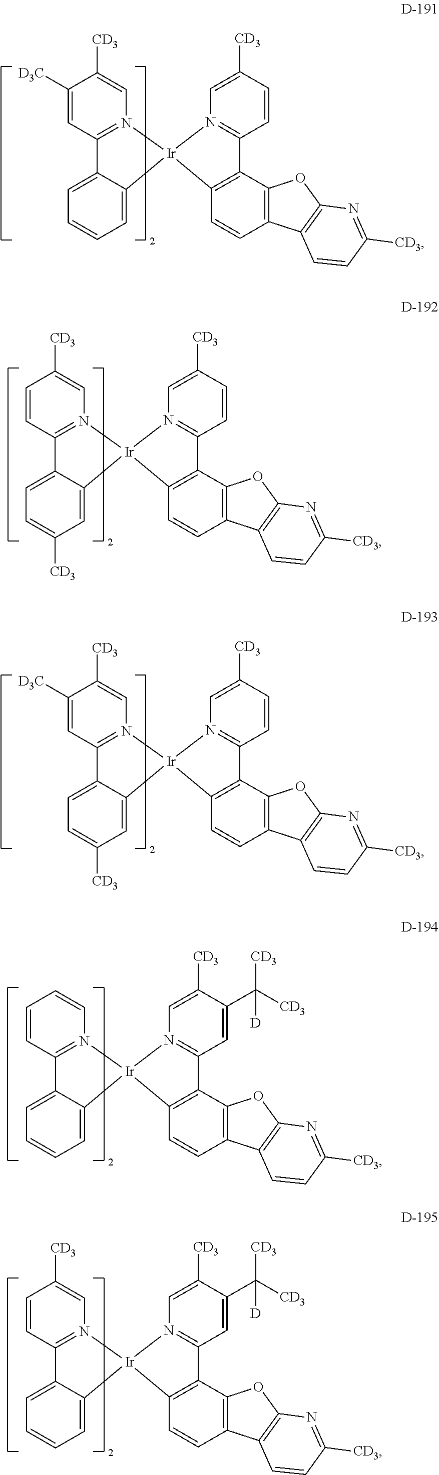

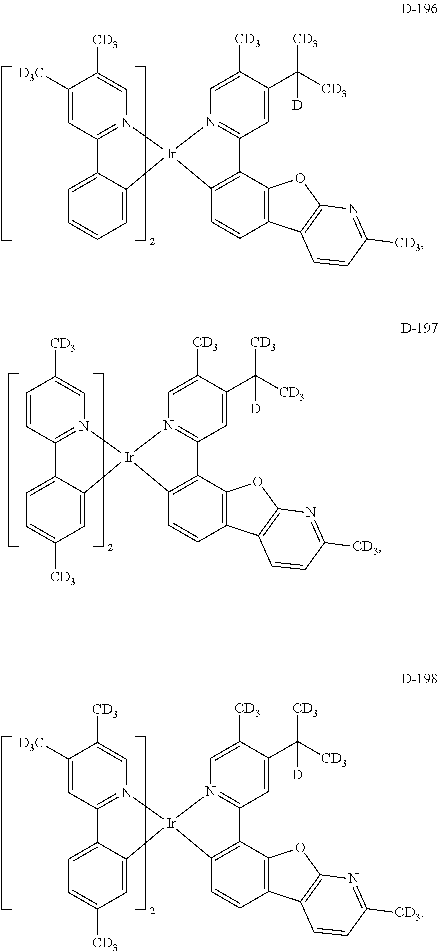

19. A display module comprising the organic electroluminescent device of claim 1.

20. A formulation comprising a host material and a dopant material, wherein the host material comprises a first host compound represented by Formula 1 and a second host compound represented by Formula 2, wherein the dopant material comprises a dopant compound represented by Formula 3: 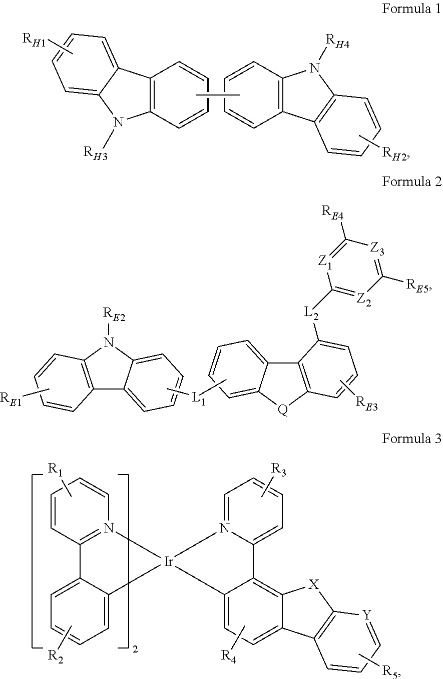
wherein
R_{H1}, R_{H2}, R_{E1}, R_{E3}, R₁ to R₅ may represent mono substitution, multiple substitutions or no substitution; when R_{H1}, R_{H2}, R_{E1}, R_{E3}, R₁ to R₅ represent multiple substitutions, each of R_{H1}, R_{H2}, R_{E1}, R_{E3}, R₁ to R₅ may be the same or different;
X and Q are each independently selected from O or S;
Z₁, Z₂, Z₃ and Y are each independently selected from CR or N;
L₁ and L₂ are each independently selected from the group consisting of a single bond, a substituted or unsubstituted arylene having 6 to 30 carbon atoms, a substituted or unsubstituted heteroarylene having 3 to 30 carbon atoms, and combinations thereof;
R₁ to R₅, R_{H1} to R_{H4}, R_{E1} to R_{E5} and R are each independently selected from the group consisting of hydrogen, deuterium, halogen, a substituted or unsubstituted alkyl group having 1 to 20 carbon atoms, a substituted or unsubstituted cycloalkyl group having 3 to 20 ring carbon atoms, a substituted or unsubstituted heteroalkyl group having 1 to 20 carbon atoms, a substituted or unsubstituted arylalkyl group having 7 to 30 carbon atoms, a substituted or unsubstituted alkoxy group having 1 to 20 carbon atoms, a substituted or unsubstituted aryloxy group having 6 to 30 carbon atoms, a substituted or unsubstituted alkenyl group having 2 to 20 carbon atoms, a substituted or unsubstituted aryl group having 6 to 30 carbon atoms, a substituted or unsubstituted heteroaryl group having 3 to 30 carbon atoms, a substituted or unsubstituted alkylsilyl group having 3 to 20 carbon atoms, a substituted or unsubstituted arylsilyl group having 6 to 20 carbon atoms, a substituted or unsubstituted amino group having 0 to 20 carbon atoms, an acyl group, a carbonyl group, a carboxylic acid group, an ester group, a nitrile group, an isonitrile group, a thiolalkyl group, a sulfinyl group, a sulfonyl group, a phosphino group, and combinations thereof;
adjacent substitutions may be optionally joined to form a ring.

21. The formulation of claim 20, further comprising at least one solvent.
